(19) United States
(12) Patent Application Publication
(10) Pub. No.: US 2023/0001017 A1
(43) Pub. Date: Jan. 5, 2023

(54) FORMULATION FOR DELIVERY OF LUBRICIN GENE

(71) Applicant: University of lowa Research Foundation, Iowa City, IA (US)

(72) Inventors: Aliasger K. Salem, Coralville, IA (US); James A. Martin, Iowa City, IA (US); Leela Raghava Jaidev Chakka, Iowa City, IA (US); Juliana c. Quarterman, Coralville, IA (US); Hongjun Zheng, St. Louis, MO (US); Dong Rim Seol, lowa City, IA (US)

(21) Appl. No.: 17/772,450

(22) PCT Filed: Oct. 28, 2020
(86) PCT No.: PCT/US2020/057750

§ 371 Date: Apr. 27, 2022

Related U.S. Application Data
(60) Provisional application No. 62/926,872, filed on Oct. 28, 2019.

(51) Int. Cl. A61K 48/00; C07K 14/47

(52) U.S. Cl. CPC A61K 48/005; A61K 48/0008; C07K 14/4713; C12N 2750/14143

(57) ABSTRACT

Compositions comprising nanoparticles, nanoplexes or virus comprising isolated nucleic acid comprising nucleic acid encoding a mammalian, and methods of using the compositions, are provided.

### CROSS-REFERENCE TO RELATED APPLICATIONS

This application claims the benefit of the filing date of U.S. application No. 62/926,872, filed on Oct. 28, 2019, the disclosure of which is incorporated by reference herein.

### STATEMENT OF GOVERNMENT RIGHTS

This invention was made with government support under W81XWH-14-1-0327 awarded by the Department of Defense. The government has certain rights in the invention.

### BACKGROUND

Joint capsule contracture and the formation of intra-articular fibrotic lesions can result in severe limitations in joint mobility (Manrique et al., 2015). Fibrosis often develops after joint trauma or major surgical procedures such as total knee arthroplasty (TKA) and anterior cruciate ligament (ACL) reconstruction (Cheuy et al., 2017; Magit et al., 2007). Approximately 3-10% of TKA patients are treated for arthrofibrosis, while 2-35% of ACL reconstruction patients and 14.5% of patients with knee intra-articular fractures require intervention (Abdul et al., 2015; Haller et al., 2015; Sanders et al., 2017). The standard-of-care is limited to arthroscopic debridement, capsule-release, and manipulating limbs under anesthesia. While these procedures can partially restore joint mobility, 25% of patients suffer recurring fibrosis that necessitates multiple interventions (Dean et al., 2016; Komuijt et al., 2018; Stephenson et al., 2010; Usher et al., 2019). Thus, there is a need to develop approaches to augment these interventions.

Transforming growth factor-beta 1 (TGF-β1) and histamines released by mast cells activate adjacent fibroblasts to migrate to wound sites, where they proliferate and transform into highly contractile myofibroblasts that rapidly close the wound (Freeman et al., 2010; Grinnell & Ho, 2002). Myofibroblasts express alpha-smooth muscle actin (α-SMA), a marker that distinguishes them from other fibroblasts (Unterhauser et al., 2004). Normally myofibroblasts undergo apoptosis at the end of tissue repair and remodeling (Darby et al., 2014). In contrast, myofibroblasts in fibrotic tissues resist apoptosis and continue to synthesize and contract the extracellular matrix (ECM), resulting in tissue stiffening (Oakley et al., 2005; Zhang et al., 2018). In addition, mechanical forces exerted by myofibroblasts can activate latent TGF-01 sequestered in the ECM (Wipff et al., 2007), which further stimulates the fibroblast to myofibroblast transformation (Van De Water et al., 2013). Moreover, substrate stretching enhances α-SMA and type I collagen expression, and induces reorganization of the ECM (Grinnell & Ho, 2002; Junker et al., 2008; Sawhney & Howard, 2004; Wakatsuki & Elson, 2003).

Capsule contracture from joint immobilization is accompanied by the proliferation of cells lining the synovial intima (Trudel et al., 2003), which is thought to contribute to capsule hypertrophy by increasing the local secretion of TGF-β1 and pro-inflammatory cytokines (User et al., 2019). Thus, thickening of the intimal cell layer is part of the pro-fibrotic response to immobilization and is of interest in assessing the effects of anti-fibrotic drugs.

Interventions targeting myofibroblasts have the potential to promote the resorption of fibrotic tissues and prevent their recurrence (Oakley et al., 2005; Wakatsuki & Elson, 2003; Atluri et al., 2016; Engel et al., 2006; Tiede et al., 2009). Drugs that promote myofibroblast apoptosis aid in the resolution of liver fibrosis in a rat model, and to promote scarless healing in incisional wounds in rats and humans (Abe et al., 2012; Kim et al., 2005). Sulfasalazine (SSZ) is an anti-inflammatory aminosalicylate drug that inhibits kappa B kinase, an activity that promotes myofibroblast apoptosis (Oakley et al., 2005; Elsharkawy et al., 2005). Cis-hydroxyproline (CHP) and beta-amino propionitrile (BAPN), drugs that block collagen deposition, also proved to be successful in halting the progression of fibrosis in various contexts including flexor tendon adhesions and bleomycin-induced lung fibrosis (Steplewski et al., 2017; McCombe et al., 2006; Canelon & Wallace2016; Ledwozyw, 1995). In addition, BAPN prevented the development of fibrosis in a rat stifle joint immobilization model (Furlow & Peacock, 1967). Neither CHP nor BAPN retarded normal wound healing at therapeutic dose levels (McCombe et al., 2006; Bulut et al., 2004).

### SUMMARY

Post-traumatic osteoarthritis (PTOA) is a debilitating condition that results in the downregulation of the lubricin gene and increased shear stress within the joint. Acute intra-articular injection of amobarbital treatment protected articular cartilage chondrocytes from the acute effects of joint injury and dramatically forestalled the progression of PTOA at 6 months post-intra-articular fracture (IAF) in the porcine model. However, moderate degenerative changes were observed at 12 months, suggesting that cellular processes other that those addressed by amobarbital contributed to long-term results.

Because amobarbital therapy alone leaves cartilage vulnerable to long-term mechanical insults resulting from lubricin loss and/or joint incongruity, cartilage was shielded from these effects by enhancing the levels of lubricin, a glycoprotein that, together with hyaluronic acid and phospholipids, accounts for the nearly frictionless motion of normal joints. However, because lubricin itself is unstable, necessitating periodic intra-articular injections to maintain joint lubrication, other delivery vehicles were tested. In one embodiment, plasmid DNA (pDNA) encoding lubricin was complexed with a cationic polymer, e.g., polyamidoamine (PAMAM) or polyethyleneimine (PEI), thereby forming nanoplexes. These nanoplexes were then encapsulated into the degradable nanoparticles, e.g., poly(lactic-co-glycolic) acid (PLGA) nanoparticles. This delivery system for the lubricin gene allows for a local and sustained release therapeutic to upregulate the production of lubricin in damaged joints and thus mitigate the negative side effects of PTOA. This delivery system may be employed with one or more anti-fibrotic drugs, e.g., sulfasalazine, beta-amino propionitrile, cis-hydroxyl proline, blebbistatin, or paclitaxel, e.g., the nanoparticles and the anti-fibrotic may be co-delivered in the same formulation, or delivered separately. This delivery system may be employed in with amobarbital or a derivative thereof, e.g., pentobarbital, secobarbital, phenobarbital, adenosine diphosphate ribose, or metformin.

In one embodiment, a composition comprising nanoparticles comprising isolated nucleic acid comprising nucleic acid encoding a mammalian lubricin and a cationic polymer is provided. In one embodiment, the nucleic acid encodes a lubricin having at least 80% amino acid sequence identity to any one of SEQ ID Nos. 1-6. In one embodiment, the nucleic acid encodes a human lubricin. In one embodiment, the cationic polymer comprises a cationic peptide, a linear or branched synthetic polymer, a polysaccharide, a natural polymer or an activated or non-activated dendrimer. In one embodiment, the cationic peptide comprises polylysine or polyornithine. In one embodiment, the cationic linear or branched synthetic polymer comprises polybrene or polyethyleneimine. In one embodiment, the cationic polysaccharide comprises cyclodextrin or chitosan. In one embodiment, the cationic polymer comprises PEI, PAMAM or chitosan, or a combination thereof.

In one embodiment, the nanoparticles are formed of lactic acid, glycolic acid, caproic acid, or combinations thereof. In one embodiment, the nanoparticles have an average diameter of about 450 nm to about 700 nm. In one embodiment, the nanoparticles have an average diameter of about 550 nm to about 650 nm. In one embodiment, the nanoparticles have an average diameter of about 100 nm to about 150 nm. In one embodiment, the nanoparticles have an average diameter of about 150 nm to about 200 nm. In one embodiment, the nanoparticles comprise a coating.

In one embodiment, the coating comprises a polysaccharide or a peptide. In one embodiment, the polysaccharide comprises chitosan. In one embodiment, the coating comprises PLL (poly(L-lysine)). In one embodiment, the composition further comprises an anti-fibrotic agent, e.g., sulfasalazine, amobarbital, beta-amino propionitrile, cis-hydroxyl proline, blebbistatin or paclitaxel.

In one embodiment, a recombinant viral vector comprising nucleic acid encoding a mammalian lubricin is provided. In one embodiment, the nucleic acid encodes a lubricin having at least 80% amino acid sequence identity to any one of SEQ ID Nos. 1-6. In one embodiment, the nucleic acid encodes a human lubricin. In one embodiment, the virus is adenovirus, adeno-associated virus, a herpesvirus, a lentivirus or a retrovirus. In one embodiment, the virus is AAV1, AAV2, AAV3, AAV5, AAV6, AAV9 or AAVrh10. In one embodiment, the virus is combined with nanoparticles, e.g., are formed of lactic acid, glycolic acid, caproic acid, or combinations thereof. In one embodiment, the nanoparticles comprise a cationic olymer.

In one embodiment, the lubricin gene is combined with a dendrimer, e.g., a dendrimer having an alkyl diamine core with tertiary amines such as PAMAM. In one embodiment, the lubricin gene is combined with nanoparticles comprising a dendrimer, e.g., a dendrimer having an alkyl diamine core with tertiary amines such as PAMAM In one embodiment, the dendrimer is PAMAM, for example, formed of an ethylenediamine, 1,4 diaminobutane, 1,6-diaminohexane, 1,12-diaminododocane or cystamine core. In one embodiment, PAMAM has surface groups that include but are not limited to amine, amidoethanol, amidoethylethanolamine, or succinamic acid. In one embodiment, the dendrimer comprises different generations of PAMAM such as, for example, generation 3, 4, 5, 6, or 7. In one embodiment, the PAMAM core comprises ethylenediamine, cystamine, diamino hexane, diaminododecane, or diaminobutane.

In one embodiment, a composition comprising nanoparticles is provided comprising isolated nucleic acid comprising nucleic acid encoding a mammalian lubricin complexed with a cationic polymer. In one embodiment, the nucleic acid encodes a lubricin having at least 80% amino acid sequence identity to any one of SEQ ID Nos. 1-6. In one embodiment, the nucleic acid encodes a human lubricin. In one embodiment, the cationic polymer comprises a cationic peptide, a linear or branched synthetic polymer, a polysaccharide, a natural polymer or an activated or non-activated dendrimer. In one embodiment, the cationic peptide comprises polylysine or polyornithine. In one embodiment, the cationic linear or branched synthetic polymer comprises polybrene or polyethyleneimine. In one embodiment, the cationic polysaccharide comprises cyclodextrin or chitosan. In one embodiment, the cationic polymer comprises PEI, PAMAM or chitosan. In one embodiment, the nanoparticles are formed of lactic acid, glycolic acid, caproic acid, or combinations thereof. In one embodiment, the nanoparticles have an average diameter of about 450 nm to about 700 nm. In one embodiment, the nanoparticles have an average diameter of about 550 nm to about 650 nm. In one embodiment, the composition further comprises an anti-fibrotic agent, e.g., sulfasalazine, amobarbital, beta-amino propionitrile, cis-hydroxyl proline, blebbistatin or paclitaxel. In one embodiment, the nanoparticles are formed of lipids.

Also provided is a recombinant viral vector comprising nucleic acid encoding a mammalian lubricin. In one embodiment, the virus is adenovirus, adeno-associated virus, a herpesvirus, a lentivirus or a retrovirus. In one embodiment, the virus is AAV1, AAV2, AAV3, AAV5, AAV6, AAV9 or AAVrh10.

In one embodiment, a method to prevent, inhibit or treat PTOA in a mammal is provided. The method includes administering to a joint space in a mammal in need thereof an effective amount of a composition comprising i) nanoparticles comprising isolated nucleic acid encoding a mammalian lubricin and a cationic polymer, ii) a recombinant viral vector comprising nucleic acid encoding a mammalian lubricin optionally in combination with nanoparticles, iii) nanoplexes comprising a cationic polymer and nucleic acid encoding a mammalian lubricin, or iv) nanoparticles comprising nucleic acid encoding a mammalian lubricin. In one embodiment, the mammal is a human. In one embodiment, the joint is an ankle, knee or shoulder joint. In one embodiment, the method includes administering amobarbital or a derivative thereof. In one embodiment, the method includes administering an anti-fibrotic drug. In one embodiment, the nucleic acid comprises a plasmid. In one embodiment, the nucleic acid comprises RNA.

In one embodiment, the nucleic acid comprises a constitutive promoter operably linked to the nucleic acid encoding the mammal lubricin. In one embodiment, nanoparticles comprising isolated nucleic acid encoding a mammalian lubricin and a cationic polymer are administered. In one embodiment, the cationic polymer comprises PEI or PAMAM. In one embodiment, a recombinant viral vector comprising nucleic acid encoding a mammalian lubricin is administered. In one embodiment, the viral vector is an AAV vector. In one embodiment, the composition further comprises nanoparticles, e.g., comprising a cationic polymer. In one embodiment, the nanoparticles comprise lactic acid, glycolic acid, or a combination thereof. In one embodiment, nanoplexes comprising a cationic polymer and nucleic acid encoding a mammalian lubricin are administered. In one embodiment, the cationic polymer comprises PEI or PAMAM. In one embodiment, nanoparticles comprising nucleic acid encoding a mammalian lubricin are administered. In one embodiment, the nanoparticles are formed of lactic acid, glycolic acid, caproic acid, or combinations thereof.

In one embodiment, a method to prevent, inhibit or treat injury to cartilage in a mammal is provided. The method includes administering to a joint space in a mammal in need thereof an effective amount of a composition comprising i) nanoparticles comprising isolated nucleic acid encoding a mammalian lubricin and a cationic polymer, ii) a recombinant viral vector comprising nucleic acid encoding a mammalian lubricin optionally in combination with nanoparticles, iii) nanoplexes comprising a cationic polymer and nucleic acid encoding a mammalian lubricin, or iv) nanoparticles comprising nucleic acid encoding a mammalian lubricin. In one embodiment, the mammal is a human. In one embodiment, the joint is an ankle, knee or shoulder joint. In one embodiment, the method includes administering amobarbital or a derivative thereof. In one embodiment, the method includes administering an anti-fibrotic drug. In one embodiment, the nucleic acid comprises a plasmid. In one embodiment, the nucleic acid comprises RNA. In one embodiment, the nucleic acid comprises a constitutive promoter operably linked to the nucleic acid encoding the mammal lubricin. In one embodiment, nanoparticles comprising isolated nucleic acid encoding a mammalian lubricin and a cationic polymer are administered. In one embodiment, the cationic polymer comprises PEI or PAMAM. In one embodiment, a recombinant viral vector comprising nucleic acid encoding a mammalian lubricin is administered. In one embodiment, the viral vector is an AAV vector. In one embodiment, the composition further comprises nanoparticles, e.g., comprising a cationic polymer. In one embodiment, the nanoparticles comprise lactic acid, glycolic acid, or a combination thereof. In one embodiment, nanoplexes comprising a cationic polymer and nucleic acid encoding a mammalian lubricin are administered. In one embodiment, the cationic polymer comprises PEI or PAMAM. In one embodiment, nanoparticles comprising nucleic acid encoding a mammalian lubricin are administered. In one embodiment, the nanoparticles are formed of lactic acid, glycolic acid, caproic acid, or combinations thereof.

Further provided is a method to treat PTOA in a mammal, comprising: administering to a joint space in a mammal in need thereof an effective amount of a composition comprising nanoparticles comprising isolated nucleic acid encoding a mammalian lubricin complexed with a cationic polymer or a recombinant viral vector comprising nucleic acid encoding a mammalian lubricin. In one embodiment, the joint is an ankle, knee or shoulder joint. In one embodiment, amobarbital or a derivative thereof is administered. In one embodiment, an anti-fibrotic drug is administered. In one embodiment, the nucleic acid comprises a plasmid. In one embodiment, the nucleic acid comprises RNA. In one embodiment, the nucleic acid comprises a constitutive promoter operably linked to the nucleic acid encoding the mammalian lubricin.

In one embodiment, a method to inhibit or treat injury to cartilage in a mammal is provided comprising administering to a joint space in a mammal in need thereof an effective amount of nanoparticles comprising isolated nucleic acid encoding a mammalian lubricin complexed with a cationic polymer or a recombinant viral vector comprising nucleic acid encoding a mammalian lubricin.

In one embodiment, the mammal is a human. In one embodiment, the joint is an ankle, knee or shoulder joint. In one embodiment, amobarbital or a derivative thereof is administered. In one embodiment, an anti-fibrotic drug is administered. In one embodiment, the nucleic acid comprises a plasmid. In one embodiment, the nucleic acid comprises RNA. In one embodiment, the nucleic acid comprises a constitutive promoter operably linked to the nucleic acid encoding the mammalian lubricin.

### BRIEF DESCRIPTION OF THE FIGURES

FIG. 1. Drop tower device for impact loading.

FIG. 2. Localized degeneration at an impact site on the medial femoral condyle. Safranin-O fast green staining reveals cartilage loss and proteoglycan depletion.

FIG. 3. Impact-induced PTOA. Automated Mankin scores for medial femoral PTOA at 8 weeks post-op in sham-operated controls, controls with destabilized menisci (DMM), and impacted (Imp) specimens.

FIG. 4. Molecular cloning of LubC (PRG4)-GFP fusion protein gene. Lub C contains a water-binding mucin-rich domain, and the cartilage-binding hemopexin-like (HP) domain, both of which are essential for boundary lubrication in cartilage. The threonine-rich amino acid sequence indicated in the mucin domain (KEPAPTT) is repeated 76 times, allowing for dense o-linked glycosylation (O-GlcNAc). The GFP gene was inserted in the mucin-rich domain in order to render the gene product easily traceable in vivo. This fusion gene was cloned into an AAV-compatible expression plasmid.

FIGS. 5A-5D. PRG4-GFP reduces friction in injured cartilage. (A) Schematic: Shear stress was applied to cartilage via a stepper motor actuator that slides a steel platen back and forth over the cartilage surface (3 mm translation) with a 1 kilogram axial load. (B) Osteochondral explants were subjected to a high energy impact to simulate joint injury prior to friction testing. Friction coefficients (p) for several materials were tested to validate the friction device (n=4) Results were similar to those reported in the literature. In particular, the coefficient for normal articular cartilage was close to zero (0.015±0.004) (C). PRG4-GFP and synovial fluid (SF) dramatically reduced friction coefficients in injured cartilage (D) (n=4-10, p<0.05, *p<0.01, and **p<0.001).

FIG. 6. Cytoprotective effect of PRG4-GFP on chondrocytes in shear-loaded cartilage. Confocal images show live cells (green) and dead cells (red) on the surface of explants after 7 days of post-impact shear loading (left). While numerous dead cells are apparent in the control explant incubated in Hanks balanced salt solution (HBSS), few dead cell were present in the explant incubated in HBSS with recombinant PRG4-GFP fusion protein (100 μg/ml). Scale bar=200 μm Viability normalized to initial viability (day 0) was significantly higher in the 20% synovial fluid (SF) and PRG4-GFP-treated groups than in the HBSS or 0.3 μM BSA (bovine serum albumin)-treated groups. (right) (n=3, *p<0.01, and ***p<0.001).

FIG. 7. Therapeutic effect of lubricin/PRG4 gene therapy on PTOA in a rabbit ACLT model. Left: Safranin-O/fast green histology of the rabbit proximal tibia indicates less proteoglycan depletion in the PRG4-GFP specimen versus the GFP specimen. Right: Lubricin/PRG4 gene therapy significantly reduced Mankin scores versus GFP control at 8 weeks post-op (right). Group sizes are indicated on the columns. p values are indicated.

FIG. 8. Confirmation of PTG4-transgene expression in synovial tissues. rtPCR results show an average 15 fold increased in treated and injured joints (ACLT+PRG4) versus untreated contralateral control (n=3).

FIG. 9. In vivo gene transfer by intra-articular injection of plasmid DNA. rtPCR results are shown for the miR-17 transcript in mice with DMM treated with PMIS-17, empty vector control (EV), or no DNA. Fold change is with respect to EV control.

FIG. 10. Overall design.

FIG. 11. Automated Mankin grading. The safranin-O stained section shows a cartilage lesion at 8 weeks after an impact. The program first segments cartilage, and then assigns scores for the various Mankin categories at 0.5 mm intervals.

FIG. 12. Porcine intra-articular-fracture model and therapeutic effects of amobarbital. Left: Creation and fixation of a single fragment fracture of the tibial plafond. Right: Average Mankin scores at 6 months post-op in anatomically-reduced joints. Experimental groups included normal, sham-operated (Sham), ORIF without treatment (ORIF), and ORIF treated with amobarbital (amo) or n-acetyl-cysteine (NAC), an antioxidant. * p<0.05 vs control, ** p<0.05 vs ORIF.

FIG. 13. Chemical structures for PAMAM generation 3 (left) and PEI (right).

FIG. 14. Gel retardation assay in 1% agarose gel containing ethidium bromide: lane 1, DNA ladder; lane 2, pDNA nanoplexes; lanes 3-6, PAMAM/pDNA nanoplexes N/P ratios 0.5, 1, 5, and 10 respectively; lanes 7-10, PAMAM/pDNA nanoplexes N/P ratios 0.5, 1, 5, and 10 respectively incubated with 5% heparin sodium salt for 15 minutes.

FIGS. 15A-15D. SEM images of blank and PAMAM/pDNA nanoplex-loaded PLGA nanoparticles made using the double emulsion, solvent evaporation technique. The nanoplexes loaded into the particles are N/P ratios 1, 5 and 10 for B, C and D respectively. Scale bars represent 5 μm.

FIG. 16. Exemplary cationic polymers.

FIG. 17. Gel retardation assay with PEI-pDNA complexes.

FIG. 18. Particle size and zeta potential of PEI-pDNA complexes.

FIG. 19. Gel retardation assay with PAMAM-pDNA complexes.

FIG. 20. Particle size and zeta potential of PAMAM-pDNA complexes.

FIG. 21. Gel retardation assay with chitosan-pDNA complexes.

FIG. 22. Particle size and zeta potential of chitosan-pDNA complexes.

FIG. 23. Nanoplex loaded PLGA.

FIG. 24. Gel retardation assay with PLGA nanoparticles and PAMAM-pDNA complexes.

FIG. 25. Particle size of PLGA nanoparticles and PAMAM N1-pDNA complexes.

FIG. 26. Particle size of PLGA nanoparticles and PAMAM N10-pDNA complexes.

FIG. 27. Particle size of PLGA nanoparticles and PAMAM N20-pDNA complexes.

FIGS. 28A-288. HEK 293T cells at 50,000 cells per well were transfected with 20 uL, 40 uL, PEI alone of control and images taken at 24 hours (A) and 48 hours (B).

FIGS. 29A-29C. Flow cytometry

FIGS. 30A-30D. Drug encapsulation in PLGA pellets. (A) Melt extrusion method for producing drug-loaded PLGA pellets and In vitro elution kinetics for BAPN (B), CHP (C), and SSZ (D). Error bars represent standard deviations based on triplicate determinations.

FIGS. 31A-31F. Immobilization leads to fibrosis of the stifle joint. (A) Medio-lateral radiograph shows a K-wire positioned to rigidly fix the stifle joint in flexion. (B) Anterior view of an immobilized rabbit stifle Joint. The femoral condyles and tibial plateau are labelled (FC and TP respectively). The arrow points to Intra-articular fibrotic tissue. Bar=1.0 cm. (C and D) H&E-stained sections of a posterior capsule from a normal joint (C) and an immobilized joint (D). (E and F) Immunohistochemical staining for alpha-SMA in posterior capsules from normal (E) and Immobilized joints (F). Bars=50 μm.

FIG. 32. Immobilization and treatment effects on synovial intimal thickness. Individual data points (dots), group means (wide horizontal bar), and standard deviations (error bars) are shown. *p=0.01. **p=0.025 by one-way ANOVA. Power of the test with p=0.05: 0.981.

FIGS. 33A-33E. Effects of Immobilization and treatment on stifle joint stiffness. Shown are typical flexion-extension plots from a normal joint (A), an immobilized joint treated with a blank pellet (control) (B), and an Immobilized joint treated with SSZ (C). (D) for maximum torque are shown. *p=0.0008, **p=0.0065 by one-way ANOVA. Power of the test with p=0.05: 0.974. (E) Group means and standard deviations for dissipated energy. *p=0.026. Power of the test with p=0.05: 0.734.

FIG. 34. Effects of treatments on ACL function. Drawer tests used to evaluate A-P laxity showed a decline in anterior stiffness in immobilized joints treated with a blank pellet (control) relative to normal (*p=0.033 by one-way ANOVA) and Increased in stiffness in the SSZ group relative to control (**p=0.011 by one ANOVA). Power of the test with p=0.05: 0.721.

FIGS. 35A-35D. Effects of immobilization and treatment on collagen gel compaction. (A) Representative Images show collagen disks after 48 hours of compaction by cells from the indicated groups. (B) The graph shows Individual data points, group means and standard deviations for maximum compaction at 48 hours. *p=0.001, **p=0.005, ***p=0.0006, by one-way ANOVA. Power of the test with p=0.05: 0.990. (C) Compaction from 1-12 hours Is plotted. (D) Compaction rates based on the slopes of the curves shown in C (means+/−standard deviations). *p=0.002 by one-way ANOVA. Power of the test with p=0.05: 0.874.

FIGS. 36A-36G. Immunofluorescence staining for α-SMA. (A) A high magnification image shows JCFs stained for α-SMA (green), polymerized actin (red), and DNA (blue). The fluorescence images show representative low magnification views of triple stained cells from a normal joint (B), from a control joint (C), and from immobilized joints treated with BAPN (D), CHP (E), or SSZ (F). Bars=100 um. (G) The box and whisker plot show median values, 25^{th} and 75^{th} percentiles, and maximum and minimum data points for percent α-SMA positive in the indicated groups. *p=0.0021, **p=0.0007 by one-way ANOVA on ranks.

FIG. 37. Pellet placement in the stifle joint. The 1 mm×4 mm pellet (red) is sutured to the patellar tendon distal to the patella.

FIG. 38. A custom-designed flexion/extension testing machine. The device equipped with a stepper motor and a 10 N load cell, and controlled by LabVIEW.

FIGS. 39A-39B. Method for measuring joint angles. The femur and tibia are rigidly fixed flexion/extension device (A). Starting angle was measured by a digital protractor (B).

FIGS. 40A-40D. Effects of drugs and PLGA on rabbit chondrocyte viability in vitro. (A-C) Viability in monolayer cultures dosed with the indicated concentrations of the drugs for 24 hours. Based on in vitro elution kinetics, star (h) indicates expected maximal daily release rates in vivo. (D) Viability in monolayer cultures exposed to the indicated masses of PLGA pellets (0, 4.3, and 9.6 mg). According to ISO guideline (#10993-5; Tests for in vitro cytotoxicity), cells were plated in the bottom of a 24-Transwell™ plate (Life Sciences), and the pellets were inserted in upper chamber for 24 hours. Viability was measured by CellTiter 96® Aqueous One Solution Cell Proliferation Assay (Promega). DMSO (10%) was included as a positive control.

FIG. 41. Schematic of exemplary preparation of cationic PLGA nanoparticles.

FIG. 42. SEM image of PLGA nanoparticles showing roughly spherical particle shape with smooth surface. FIG. 43.

FIGS. 43A-43E. The cell proliferation (panels A, C and E) and transfection efficiency by flow cytometer (panels B and D) of cationic nanoparticle complexed with pEGFP in HEK293T (panels A and B), Rabbit synovial fibroblasts (panels C and D) and Bovine chondrocytes (panel E). The data were represented mean*SD.

FIGS. 44A-44B. A) The confocal microscopy images of porcine cartilage samples treated with different pEGFP and AAV complexed PLGA-PEI and appropriate controls. The red color represents the rhodamine B) fluorescence of PLGA-PEI, the green color is the pEGFP expression and blue color is DAPI staining of nucleus. B) Quantitative analysis of fluorescence expressed in the confocal microscopy for different groups in the deep and superficial zones.

FIGS. 45A-45C. A) The image showing the impact apparatus that delivered the appropriate J/cm2 where there is a clear break in the explant at 2.5 J/cm2 and the explant is intact at 2 J/cm2. B) Confocal microscopy images of impact treated explants provided with nanoparticles incubated with and without pEGFP and AAV. The red color is the rhodamineB fluorescence of PLGA-PEI and PLGA-PAMAM nanoparticles, green color is the expression of pEGFP and blue color is the DAPI stain of nucleus. C) Quantified values of the fluorescence from the confocal images. The data represent n=3, mean*SD.

FIGS. 46A-46B. A) Immunohistochemistry (IHC) of lubricin (red color) in rabbit cartilage. B) The graph plot represents the thickness calculated from the lubricin IHC images obtained from rabbit cartilages treated with associated adenovirus (AAV) and non-viral transfection agents complexed with EGFP and lubricin plasmids. The data quantified for at least 5 samples was represented as mean±standard deviation.

### DETAILED DESCRIPTION

### Definitions

A “vector” refers to a macromolecule or association of macromolecules that comprises or associates with a polynucleotide, and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a “target polynucleotide” or “transgene,” may comprise a coding sequence of interest in gene therapy (such as a gene encoding a protein of therapeutic interest), a coding sequence of interest in vaccine development (such as a polynucleotide expressing a protein, polypeptide or peptide suitable for eliciting an immune response in a mammal), and/or a selectable or detectable marker.

“Transduction,” “transfection,” “transformation” or “transducing” as used herein, are terms referring to a process for the introduction of an exogenous polynucleotide into a host cell leading to expression of the polynucleotide, e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by heterologousization assays, e.g., Northern blots, Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene delivery” refers to the introduction of an exogenous polynucleotide into a cell for gene transfer, and may encompass targeting, binding, uptake, transport, localization, replicon integration and expression.

“Gene transfer” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

An “infectious” virus or viral particle is one that comprises a polynucleotide component which it is capable of delivering into a cell for which the viral species is trophic. The term does not necessarily imply any replication capacity of the virus.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment described herein that is a polynucleotide encompasses both the double-stranded form and each of two complementary single-stranded forms known or predicted to make up the double-stranded form.

An “isolated” polynucleotide, e.g., plasmid, virus, polypeptide or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Isolated nucleic acid, peptide or polypeptide is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (e.g., the molecule may single-stranded), but may contain both the sense and anti-sense strands (i.e., the molecule may be double-stranded). Enrichment can be measured on an absolute basis, such as weight per volume of solution, or it can be measured in relation to a second, potentially interfering substance present in the source mixture. Increasing enrichments of the embodiments of this invention are envisioned. Thus, for example, a 2-fold enrichment, 10-fold enrichment, 100-fold enrichment, or a 1000-fold enrichment.

A “transcriptional regulatory sequence” refers to a genomic region that controls the transcription of a gene or coding sequence to which it is operably linked. Transcriptional regulatory sequences of use in the present invention generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from the entity to which it is compared. For example, a polynucleotide introduced by genetic engineering techniques into a different cell type is a heterologous polynucleotide (and, when expressed, can encode a heterologous polypeptide). Similarly, a transcriptional regulatory element such as a promoter that is removed from is native coding sequence and operably linked to a different coding sequence is a heterologous transcriptional regulatory element.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present invention are provided below.

“Host cells,” “cell lines,” “cell cultures,” “packaging cell line” and other such terms denote higher eukaryotic cells, such as mammalian cells including human cells, useful in the present invention, e.g., to produce recombinant virus or recombinant fusion polypeptide. These cells include the progeny of the original cell that was transduced. It is understood that the progeny of a single cell may not necessarily be completely identical (in morphology or in genomic complement) to the original parent cell.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include AAV promoters, e.g., P5, P19, P40 and AAV ITR promoters, as well as heterologous promoters.

An “expression vector” is a vector comprising a region which encodes a gene product of interest, and is used for effecting the expression of the gene product in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphonylation, lipidation, or conjugation with a labeling component.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells, or is otherwise flanked by a different nucleic acid sequence than that found in nature, e.g., an expression cassette which links a promoter from one gene to an open reading frame for a gene product from a different gene.

“Transformed” or “transgenic” is used herein to include any host cell or cell line, which has been altered or augmented by the presence of at least one recombinant DNA sequence. The host cells of the present invention are typically produced by transfection with a DNA sequence in a plasmid expression vector, as an isolated linear DNA sequence, or infection with a recombinant viral vector.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less e.g., with 2 bases or less. When using oligonucleotides as probes or treatments, the sequence homology between the target nucleic acid and the oligonucleotide sequence is generally not less than 17 target base matches out of 20 possible oligonucleotide base pair matches (85%); not less than 9 matches out of 10 possible base pair matches (90%), or not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less or with 2 or less. Alternatively, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more homologous if their amino acids are greater than or equal to 50% identical when optimally aligned using the ALIGN program.

The term “corresponds to” is used herein to mean that a polynucleotide sequence is structurally related to all or a portion of a reference polynucleotide sequence, or that a polypeptide sequence is structurally related to all or a portion of a reference polypeptide sequence, e.g., they have at least 80%, 85%, 90%, 95% or more, e.g., 99% or 100%, sequence identity. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, e.g., at least 90 to 95 percent sequence identity, or at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

“Conservative” amino acid substitutions are, for example, aspartic-glutamic as polar acidic amino acids; lysine/arginine/histidine as polar basic amino acids; leucine/isoleucine/methionine/valine/alanine/glycine/proline as non-polar or hydrophobic amino acids; serine/threonine as polar or uncharged hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting polypeptide. Whether an amino acid change results in a functional polypeptide can readily be determined by assaying the specific activity of the polypeptide. Naturally occurring residues are divided into groups based on common side-chain properties: (1) hydrophobic: norleucine, met, ala, val, leu, ile; (2) neutral hydrophilic: cys, ser, thr; (3) acidic: asp, glu; (4) basic: asn, gin, his, lys, arg; (5) residues that influence chain orientation: gly, pro; and (6) aromatic; trp, tyr, phe.

The disclosure also envisions polypeptides with non-conservative substitutions. Non-conservative substitutions entail exchanging a member of one of the classes described above for another.

### Exemplary Lubricin Sequences

Sequences encoding the following exemplary lubricins may be employed in the compositions and methods described herein:

(SEQ ID NO: 1)
mawktlplyl lllisvfviq qvssqdlssc agrcgegysr datcncdync qhymeccpdf krvctaelsc kgrcfesfer grecdcdaqc
kkydkccpdy esfcaevhnp tsppsskkap ppsgasqtik sttkrspkpp nkkktkkvie seeiteehsv senqesssss ssssssstir
kikssknsaa nrelqkklkv kdnkknrtkk kptpkppvvd eagsgldngd fkvttpdist tqhnkvstsp kittakpinp rpslppnsdt
sketsltvnk ettvetkett ttnkqtstdg kekttsaket qsiektsakd laptskvlak ptpkaetttk gpalttpkep tpttpkepas
ttpkeptptt iksapttpke paptttksap ttpkepaptt tkepapttpk epaptttkep aptttksapt tpkepapttp kkpapttpke
papttpkept pttpkepapt tkepapttpk epaptapkkp apttpkepap ttpkepaptt tkepspttpk epaptttksa ptttkepapt
ttksapttpk epsptttkep apttpkepap ttpkkpaptt pkepapttpk epaptttkkp apttpkepap ttpketaptt pkkltpttpe
klapttpekp apttpeelap ttpeeptptt peepapttpk aaapntpkep apttpkepap ttpkepaptt pketapttpk gtapttlkep
apttpkkpap kelaptttke ptsttsdkpa pttpkgtapt tpkepapttp kepapttpkg tapttlkepa pttpkkpapk elaptttkgp
tsttsdkpap ttpketaptt pkepapttpk kpapttpetp ppttsevstp tttkepttih kspdestpel saeptpkale nspkepgvpt
tktpaatkpe mtttakdktt erdlrttpet ttaapkmtke tatttektte skitatttqv tstttqdttp fkittlkttt lapkvtttkk
tittteimnk peetakpkdr atnskattpk pqkptkapkk ptstkkpktm prvrkpkttp tprkmtstmp elnptsrlae amlqtttrpn
qtpnsklvev npksedagga egetphmllr phvfmpevtp dmdylprvpn qgliinpmls detnicngkp vdglttlrng tlvafrghyf
wmlspfspps parritevwg ipspidtvft rencegktff fkdsqywrft ndikdagypk pifkgfgglt gqivaalsta kyknwpesvy
ffkrggsiqq yiykqepvqk cpgrrpalny pvygettqvr rrrferaigp sqthtiriqy sparlayqdk gvlhnevkvs ilwrglpnvv
tsaislpnir kpdgydyyaf skdqyynidv psrtaraitt rsgqtlskvw yncp;
(SEQ ID NO: 2)
mawktlpiyl lllisvfviq qvssqdlssc agrcgegysr datcncdync qhymeccpdf krvctaelsc kgrcfesfer grecdedaqc
kkydkccpdy esfcaevkdn kknrtkkkpt pkppvvdeag sgldngdfkv ttpdtsttqh nkvstspkit takpinprps lppnsdiske
tsltvnkett vetkettttn kqtstdgkek ttsaketqsi ektsakdlap tskvlakptp kaetttkgpa lttpkeptpt tpkepasttp
keptpttiks apttpkepap tttksapttp kepaptttke papttpkepa ptttkepapt ttksapttpk epapttpkkp apttpkepap
ttpkeptptt pkepapttke papttpkepa ptapkkpapt tpkepapttp kepaptttke pspttpkepa ptttksaptt tkepaptttk
sapttpkeps ptttkepapt tpkepapttp kkpapttpke papttpkepa ptttkkpapt tpkepapttp ketapttpkk ltpttpekla
pttpekpapt tpeelapttp eeptpttpee papttpkaaa pntpkepapt tpkepapttp kepapttpke tapttpkgta pttlkepapt
tpkkpapkel aptttkepts ttsdkpaptt pkgtapttpk epapttpkep apttpkgtap ttlkepaptt pkkpapkela ptttkgptst
tsdkpapttp ketapttpke papttpkkpa pttpetpppt tsevstpttt kepttihksp destpelsae ptpkalensp kepgvpttkt
paatkpemtt takdktterd lrttpettta apkmtketat ttektteski tatttqvtst ttqdttpfki ttlktttlap kvtttkktit
tteimnkpee takpkdratn skattpkpqk ptkapkkpts tkkpktmprv rkpkttptpr kmtstmpeln ptsriaeaml qtttrpnqtp
nsklvevnpk sedaggaege tphmllrphv fmpevtpdmd ylprvpnqgi iinpmlsdet nicngkpvdg lttlrngtlv afrghyfwml
spfsppspar ritevwgips pidtvftrcn cegktfffkd sqywrftndi kdagypkpif kgfggltgqi vaalstakyk nwpesvyffk
rggsiqqyly kqepvqkcpg rrpalnypvy gettqvrrrr feraigpsqt htiriqyspa rlayqdkgvl hnevkvsilw rglpnvvtsa
islpnirkpd gydyyafskd qyynidvpsr taraittrsg qtlskvwync p;
(SEQ ID NO: 3)
mawktlpiyl lllisvfviq qvssqelsck grcfesferg recdcdaqck kydkccpdye sfcaevkdnk knrtkkkptp kppvvdeags
gldngdfkvt tpdtsttghn kvstspkitt akpinprpsl ppnsdtsket sltvnkettv etkettttnk qtstdgkekt tsaketqsie
ktsakdlapt skvlakptpk aetttkgpal ttpkeptptt pkepasttpk eptpttiksa pttpkepapt ttksapttpk epaptttkep
apttpkepap tttkepaptt tksapttpke papttpkkpa pttpkepapt tpkeptpttp kepapttkep apttpkepap tapkkpaptt
pkepapttpk epaptttkep spttpkepap tttksapttt kepaptttks apttpkepsp tttkepaptt pkepapttpk kpapttpkep
apttpkepap tttkkpaptt pkepapttpk etapttpkkl tpttpeklap ttpekpaptt peelapttpe eptpttpeep apttpkaaap
ntpkepaptt pkepapttpk epapttpket apttpkgtap ttlkepaptt pkkpapkela ptttkeptst tsdkpapttp kgtapttpke
papttpkepa pttpkgtapt tlkepapttp kkpapkelap tttkgptstt sdkpapttpk etapttpkep apttpkkpap ttpetppptt
sevstptttk epttihkspd estpelsaep tpkalenspk epgvpttktp aatkpemttt akdktterdl rttpetttaa pkmtketatt
tektteskit atttqvtstt tqdttpfkit tlktttlapk vittkktitt teimnkpeet akpkdratns kattpkpqkp tkapkkptst
kkpktmprvr kpkttptprk mtstmpelnp tsriaeamlq tttrpnqtpn sklvevnpks edaggaeget phmllrphvf mpevtpdmdy
lprvpnqgii inpmlsdetn icngkpvdgl ttlrngtlva frghyfwmls pfsppsparr itevwgipsp idtvftrcnc egktfffkds
qywrftndik dagypkpifk gtggltgqiv aalstakykn wpesvyffkr ggsiqqyiyk qepvqkcpgr rpalnypvyg ettqvrrrrf
eraigpsqth tiriqyspar layqdkgvlh nevkvsilwr glpnvvtsai slpnirkpdg ydyyafskdq yynidvpsrt araittrsgq
tlskvwyncp;
(SEQ ID NO: 4)
mawktlpiyl lllisvfviq qvssqdlssc agrcgegysr datcncdync qhymeccpdf krvctaelsc kgrcfesfer grecdedaqc
kkydkccpdy esfcaevhnp tsppsskkap ppsgasqtik sttkrspkpp nkkktkkvie seeiteehsv senqesssss ssssssstir
kikssknsaa nrelqkklkv kdnkknrtkk kptpkppvvd eagsgldngd fkvttpdtst tqhnkvstsp kittakpinp rpslppnsdt
sketsltvnk ettvetkett ttnkqtstdg kekttsaket qsiektsakd laptskvlak ptpkaetttk gpalttpkep tpttpkepas
ttpkeptptt iksapttpke paptttksap ttpkepaptt tkepapttpk epaptttkep aptttksapt tpkepapttp kkpapttpke
papttpkept pttpkepapt tkepapttpk epaptapkkp apttpkepap ttpkepaptt tkepspttpk epaptttksa ptttkepapt
ttksapttpk epsptttkep apttpkepap ttpkkpaptt pkepapttpk epaptttkkp apttpkepap ttpketaptt pkkltpttpe
klapttpekp apttpeelap ttpeeptptt peepapttpk aaapntpkep apttpkepap ttpkepaptt pketapttpk gtapttlkep
apttpkkpap kelaptttke ptsttsdkpa pttpkgtapt tpkepapttp kepapttpkg tapttlkepa pttpkkpapk elaptttkgp
tsttsdkpap ttpketaptt pkepapttpk kpapttpetp ppttsevstp tttkepttih kspdestpel saeptpkale nspkepgvpt
tktpaatkpe mtttakdktt erdlrttpet ttaapkmtke tatttektte skitatttqv tstttqdttp fkittlkttt lapkvtttkk
tittteimnk peetakpkdr atnskattpk pqkptkapkk ptstkkpktm prvrkpkttp tprkmtstmp elnptsriae amlqtttrpn
qtpnsklvev npksedagga egetphmllr phvfmpevtp dmdylprvpn qgiiinpmls detnicngkp vdglttlrng tlvafrghyf
wmlspfspps parritevwg ipspidtvft rcncegktff fkdsqywrft ndikdagypk pifkgfgglt gqivaalsta kyknwpesvy
ffkrggsiqq yiykqepvqk cpgrrpalny pvygettqvr rrrferaigp sqthtiriqy sparlayqdk gvlhnevkvs ilwrglpnvv
tsaislpnir kpdgydyyaf skdqyynidv psrtaraitt rsgqtlskvw yncp;
(SEQ ID NO: 5)
mawktlpiyl lllisvfviq qvssqdlssc agrcgegysr datcncdync qhymeccpdf krvctaelsc kgrcfesfer grecdcdaqc
kkydkccpdy esfcaevhnp tsppsskkap ppsgasqtik sttkrspkpp nkkktkkvie seeitevkdn kknrtkkkpt pkppvvdeag
sgldngdfkv ttpdtsttqh nkvstspkit takpinprps lppnsdtske tsltvnkett vetkettttn kqtstdgkek ttsaketqsi
ektsakdlap tskvlakptp kaetttkgpa lttpkeptpt tpkepasttp keptpttiks apttpkepap tttksapttp kepaptttke
papttpkepa ptttkepapt ttksapttpk epapttpkkp apttpkepap ttpkeptptt pkepapttke papttpkepa ptapkkpapt
tpkepapttp kepaptttke pspttpkepa ptttksaptt tkepaptttk sapttpkeps ptttkepapt tpkepapttp kkpapttpke
papttpkepa ptttkkpapt tpkepapttp ketapttpkk ltpttpekla pttpekpapt tpeelapttp eeptpttpee papttpkaaa
pntpkepapt tpkepapttp kepapttpke tapttpkgta pttlkepapt tpkkpapkel aptttkepts ttsdkpaptt pkgtapttpk
epapttpkep apttpkgtap ttlkepaptt pkkpapkela ptttkgptst tsdkpapttp ketapttpke papttpkkpa pttpetpppt
tsevstpttt kepttihksp destpelsae ptpkalensp kepgvpttkt paatkpemtt takdktterd lrttpettta apkmtketat
ttektteski tatttqvtst ttqdttpfki ttlktttlap kvtttkktit tteimnkpee takpkdratn skattpkpqk ptkapkkpts
tkkpktmprv rkpkttptpr kmtstmpeln ptsriaeaml qtttrpnqtp nsklvevnpk sedaggaege tphmllrphv fmpevtpdmd
ylprvpnqgi iinpmlsdet nicngkpvdg lttlrngtlv afrghyfwml spfsppspar ritevwgips pidtvftrcn cegktfffkd
sqywrftndi kdagypkpif kgfggltgqi vaalstakyk nwpesvyffk rggsiqqyiy kqepvqkcpg rrpalnypvy gettqvrrrr
feraigpsqt htiriqyspa rlayqdkgvl hnevkvsilw rglpnvvtsa islpnirkpd gydyyafskd qyynidvpsr taraittrsg
qtlskvwync p;
(SEQ ID NO: 6)
mawktlpiyl lllisvfviq qvssqelsck grcfesferg recdcdaqck kydkccpdye sfcaevhnpt sppsskkapp psgasqtiks
ttkrspkppn kkktkkvies eeiteehsvs enqessssss sssssstirk ikssknsaan relqkklkvk dnkknrtkkk ptpkppvvde
agsgldngdf kvttpdtstt qhnkvstspk ittakpinpr pslppnsdts ketsltvnke ttvetkettt tnkqtstdgk ekttsaketq
siektsakdl aptskvlakp tpkaetttkg palttpkept pttpkepast tpkeptptti ksapttpkep aptttksapt tpkepapttt
kepapttpke paptttkepa ptttksaptt pkepapttpk kpapttpkep apttpkeptp ttpkepaptt kepapttpke paptapkkpa
pttpkepapt tpkepapttt kepspttpke paptttksap tttkepaptt tksapttpke psptttkepa pttpkepapt tpkkpapttp
kepapttpke paptttkkpa pttpkepapt tpketapttp kkltpttpek lapttpekpa pttpeelapt tpeeptpttp eepapttpka
aapntpkepa pttpkepapt tpkepapttp ketapttpkg tapttlkepa pttpkkpapk elaptttkep tsttsdkpap ttpkgtaptt
pkepapttpk epapttpkgt apttlkepap ttpkkpapke laptttkgpt sttsdkpapt tpketapttp kepapttpkk papttpetpp
pttsevstpt ttkepttihk spdestpels aeptpkalen spkepgvptt ktpaatkpem tttakdktte rdlrttpett taapkmtket
atttekttes kitatttqvt stttqdttpf kittlktttl apkvtttkkt ittteimnkp eetakpkdra tnskattpkp qkptkapkkp
tstkkpktmp rvrkpkttpt prkmtstmpe lnptsriaea mlqtttrpnq tpnsklvevn pksedaggae getphmllrp hvfmpevtpd
mdylprvpnq giiinpmlsd etnicngkpv dglttlrngt lvafrghyfw mlspfsppsp arritevwgi pspidtvftr cncegktfff
kdsqywrftn dikdagypkp ifkgfggltg qivaalstak yknwpesvyf fkrggsiqqy iykqepvqkc pgrrpalnyp vygettqvrr
rrferaigps qthtiriqys parlayqdkg vlhnevkvsi lwrglpnvvt saislpnirk pdgydyyafs kdqyynidvp srtaraittr
sgqtiskvwy ncp;

or a polypeptide having at least 80%, 81%, 82%, 83%, 84%, 85%, 86%, 87%, 88%, 89%, 90%, 91%, 92%, 93%, 94%, 95%, 96%, 97%, 98%, or 99% amino acid sequence identity thereto.

### Gene Delivery Vectors

Gene delivery vectors include, for example, viral vectors, liposomes and other lipid-containing complexes, such as lipoplexes (DNA and cationic lipids), polyplexes, e.g., DNA complexed with cationic polymers such as polyethylene glycol, nanoparticles, e.g., magnetic inorganic nanoparticles that bind or are functionalized to bind DNA such as Fe₃O₄ or MnO₂ nanoparticles, microparticles, e.g., formed of polylactide polygalactide reagents, nanotubes, e.g., silica nanotubes, and other macromolecular complexes capable of mediating delivery of a gene to a host cell. Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector by the cell; components that influence localization of the transferred gene within the cell after uptake (such as agents mediating nuclear localization); and components that influence expression of the gene. Such components also might include markers, such as detectable and/or selectable markers that can be used to detect or select for cells that have taken up and are expressing the nucleic acid delivered by the vector. A large variety of such vectors are known in the art and are generally available.

Gene delivery vectors within the scope of the invention include, but are not limited to, isolated nucleic acid, e.g., plasmid-based vectors which may be extrachromosomally maintained, and viral vectors, e.g., recombinant adenovirus, retrovirus, lentivirus, herpesvirus, poxvirus, papilloma virus, or adeno-associated virus, including viral and non-viral vectors which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes. Exemplary viral gene delivery vectors are described below. Gene delivery vectors may be administered via any route including, but not limited to, intracranial, intrathecal, intramuscular, buccal, rectal, intravenous or intracoronary administration, and transfer to cells may be enhanced using electroporation and/or iontophoresis, and/or scaffolding such as extracellular matrix or hydrogels, e.g., a hydrogel patch. In one embodiment, a permeation enhancer is not employed to enhance indirect delivery to the CNS.

### Retroviral Vectors

Retroviral vectors exhibit several distinctive features including their ability to stably and precisely integrate into the host genome providing long-term transgene expression. These vectors can be manipulated ex vivo to eliminate infectious gene particles to minimize the risk of systemic infection and patient-to-patient transmission. Pseudotyped retroviral vectors can alter host cell tropism.

### Lentiviruses

Lentiviruses are derived from a family of retroviruses that include human immunodeficiency virus and feline immunodeficiency virus. However, unlike retroviruses that only infect dividing cells, lentiviruses can infect both dividing and nondividing cells. For instance, lentiviral vectors based on human immunodeficiency virus genome are capable of efficient transduction of cardiac myocytes in vivo. Although lentiviruses have specific tropisms, pseudotyping the viral envelope with vesicular stomatitis virus yields virus with a broader range (Schnepp et al., Meth. Mol. Med., 69:427 (2002)).

### Adenoviral Vectors

Adenoviral vectors may be rendered replication-incompetent by deleting the early (E1A and E1B) genes responsible for viral gene expression from the genome and are stably maintained into the host cells in an extrachromosomal form. These vectors have the ability to transfect both replicating and nonreplicating cells and, in particular, these vectors have been shown to efficiently infect cardiac myocytes in vivo, e.g., after direction injection or perfusion. Adenoviral vectors have been shown to result in transient expression of therapeutic genes in vivo, peaking at 7 days and lasting approximately 4 weeks. The duration of transgene expression may be improved in systems utilizing neural specific promoters. In addition, adenoviral vectors can be produced at very high titers, allowing efficient gene transfer with small volumes of virus.

### Adeno-Associated Virus Vectors

Recombinant adeno-associated viruses (rAAV) are derived from nonpathogenic parvoviruses, evoke essentially no cellular immune response, and produce transgene expression lasting months in most systems. Moreover, like adenovirus, adeno-associated virus vectors also have the capability to infect replicating and nonreplicating cells and are believed to be nonpathogenic to humans. Moreover, they appear promising for sustained cardiac gene transfer (Hoshijima et al., Nat. Med., 8:864 (2002); Lynch et al., Circ. Res., 80:197 (1997)).

AAV vectors include but are not limited to AAV1, AAV2, AAV5, AAV7, AAV8, AAV9 or AAVrh.10.

### Plasmid DNA Vectors

Plasmid DNA is often referred to as “naked DNA” to indicate the absence of a more elaborate packaging system. Direct injection of plasmid DNA to myocardial cells in vivo has been accomplished. Plasmid-based vectors are relatively nonimmunogenic and nonpathogenic, with the potential to stably integrate in the cellular genome, resulting in long-term gene expression in postmitotic cells in vivo. For example, expression of secreted angiogenesis factors after muscle injection of plasmid DNA, despite relatively low levels of focal transgene expression, has demonstrated significant biologic effects in animal models and appears promising clinically (Isner, Nature, 415:234 (2002)). Furthermore, plasmid DNA is rapidly degraded in the blood stream; therefore, the chance of transgene expression in distant organ systems is negligible. Plasmid DNA may be delivered to cells as part of a macromolecular complex, e.g., a liposome or DNA-protein complex, and delivery may be enhanced using techniques including electroporation.

### Exemplar Formulations

The disclosed nanoparticles may include or may be formed from biodegradable polymeric molecules which may include, but are not limited to polylactic acid (PLA), polyglycolic acid (PGA), co-polymers of PLA and PGA (e.g., polyactic-co-glycolic acid (PLGA)), poly-ε-caprolactone (PCL), polyethylene glycol (PEG), poly(3-hydroxybutyrate), poly(p-dioxanone), polypropylene fumarate, poly(orthoesters), polyol/diketene acetals addition polymers, poly-alkyl-cyano-acrylates (PAC), poly(sebacic anhydride) (PSA), poly(carboxybiscarboxyphenoxyphenoxy hexone (PCPP) poly[bis (p-carboxypheonoxy)methane](PCPM), copolymers of PSA, PCPP and PCPM, poly(amino acids), poly(pseudo amino acids), polyphosphazenes, derivatives of poly[(dichloro)phosphazenes] and poly[(organo)phosphazenes], poly-hydroxybutyric acid, or S-caproic acid, elastin, or gelatin. (See, e.g., Kumari et al., Colloids and Surfaces B: Biointerfaces 75 (2010) 1-18; and U.S. Pat. Nos. 6,913,767; 6,884,435; 6,565,777; 6,534,092; 6,528,087; 6,379,704; 6,309,569; 6,264,987; 6,210,707; 6,090,925; 6,022,564; 5,981,719; 5,871,747; 5,723,269; 5,603,960; and 5,578,709; and U.S. Published Application No. 2007/0081972; and International Application Publication Nos. WO 2012/115806; and WO 2012/054425; the contents of which are incorporated herein by reference in their entireties).

The disclosed cationic nanoparticles may be prepared by methods known in the art. (See, e.g., Nagavarma et al., Asian J. of Pharma. And Clin. Res., Vol 5, Suppl 3, 2012, pages 16-23; Cismaru et al., Rev. Roum. Chim., 2010, 55(8), 433-442; and International Application Publication Nos. WO 2012/115806; and WO 2012/054425; the contents of which are incorporated herein by reference in their entireties). Suitable methods for preparing the nanoparticles may include methods that utilize a dispersion of a preformed polymer, which may include but are not limited to solvent evaporation, nanoprecipitation, emulsification/solvent diffusion, salting out, dialysis, and supercritical fluid technology. In some embodiments, the nanoparticles may be prepared by forming a double emulsion (e.g., water-in-oil-in-water) and subsequently performing solvent-evaporation. The nanoparticles obtained by the disclosed methods may be subjected to further processing steps such as washing and lyophilization, as desired. Optionally, the nanoparticles may be combined with a preservative (e.g., trehalose).

Typically, the nanoparticles have a mean effective diameter of less than 1 micron, e.g., the nanoparticles have a mean effective diameter of between about 25 nm and about 500 nm, e.g., between about 50 nm and about 250 nm, about 100 nm to about 150 nm, about 150 nm to about 175 nm, about 150 nm to about 200 nm, about 400 nm to about 450 nm, or about 450 nm to 650 nm. The size of the particles (e.g., mean effective diameter) may be assessed by known methods in the art, which may include but are not limited to transmission electron microscopy (TEM), scanning electron microscopy (SEM), Atomic Force Microscopy (AFM), Photon Correlation Spectroscopy (PCS), Nanoparticle Surface Area Monitor (NSAM), Condensation Particle Counter (CPC), Differential Mobility Analyzer (DMA), Scanning Mobility Particle Sizer (SMPS), Nanoparticle Tracking Analysis (NTA), X-Ray Diffraction (XRD), Aerosol Time of Flight Mass Spectroscopy (ATFMS), and Aerosol Particle Mass Analyzer (APM).

The disclosed cationic biodegradable nanoparticles may have a zeta-potential that facilitates uptake by a target cell. Typically, the nanoparticles have a zeta-potential greater than 0. In some embodiments, the nanoparticles have a zeta-potential between about 5 mV to about 45 mV, between about 15 mV to about 35 mV, or between about 20 mV and about 40 mV. Zeta-potential may be determined via characteristics that include electrophoretic mobility or dynamic electrophoretic mobility. Electrokinetic phenomena and electroacoustic phenomena may be utilized to calculate zeta-potential.

In one embodiment, a non-viral delivery vehicle comprises polymers including but not limited to poly(lactic-co-glycolic acid) (PLGA), polylactic acid (PLA), linear and/or branched PEI with differing molecular weights (e.g., 2, 22 and 25 kDa), dendrimers such as polyamidoamine (PAMAM) and polymethoacrylates; lipids including but not limited to cationic liposomes, cationic emulsions, DOTAP, DOTMA, DMRIE, DOSPA, distearoylphosphatidylcholine (DSPC), DOPE, or DC-cholesterol; peptide based vectors including but not limited to Poly-L-lysine or protamine; or poly(β-amino ester), chitosan, PEI-polyethylene glycol, PEI-mannose-dextrose, DOTAP-cholesterol or RNAiMAX.

In one embodiment, the delivery vehicle is a glycopolymer-based delivery vehicle, poly(glycoamidoamine)s (PGAAs), that have the ability to complex with various polynucleotide types and form nanoparticles. These materials are created by polymerizing the methylester or lactone derivatives of various carbohydrates (D-glucarate (D), meso-galactarate (G), D-mannarate (M), and L-tartarate (T)) with a series of oligoethyleneamine monomers (containing between 1-4 ethylenamines (Liu and Reineke, 2006). A subset composed of these carbohydrates and four ethyleneamines in the polymer repeat units yielded exceptional delivery efficiency.

In one embodiment, the delivery vehicle comprises polyethyleneimine (PEI), Polyamidoamine (PAMAM), PEI-PEG, PEI-PEG-mannose, dextran-PEI, OVA conjugate, PLGA microparticles, or PLGA microparticles coated with PAMAM, or any combination thereof. The disclosed cationic polymer may include, but are not limited to, polyamidoamine (PAMAM) dendrimers. Polyamidoamine dendrimers suitable for preparing the presently disclosed nanoparticles may include 3rd-, 4th-, 5th-, or at least 6th-generation dendrimers.

In one embodiment, the delivery vehicle comprises a cationic lipid, e.g., N-[1-(2,3-dioleoyloxy)propel]-N,N,N-trimethylammonium (DOTMA), 2,3-dioleyloxy-N-[2-spermine carboxamide]ethyl-N,N-dimethyl-1-propanammonium trifluoracetate (DOSPA, Lipofectamine); 1,2-dioleoyl-3-trimethylammonium-propane (DOTAP); N-[1-(2,3-dimyristloxy) propyl]; N,N-dimethyl-N-(2-hydroxyethyl) ammonium bromide (DMRIE), 3-β-[N—(N,N′-dimethylaminoethane) carbamoyl] cholesterol (DC-Chol); dioctadecyl amidoglyceryl spermine (DOGS, Transfectam); or imethyldioctadeclyammonium bromide (DDAB). The positively charged hydrophilic head group of cationic lipids usually consists of monoamine such as tertiary and quaternary amines, polyamine, amidinium, or guanidinium group. A series of pyridinium lipids have been developed (Zhu et al., 2008; van der Woude et al., 1997; Ilies et al., 2004). In addition to pyridinium cationic lipids, other types of heterocyclic head group include imidazole, piperizine and amino acid. The main function of cationic head groups is to condense negatively charged nucleic acids by means of electrostatic interaction to slightly positively charged nanoparticles, leading to enhanced cellular uptake and endosomal escape.

Lipids having two linear fatty acid chains, such as DOTMA, DOTAP and SAINT-2, or DODAC, may be employed as a delivery vehicle, as well as tetraalkyl lipid chain surfactant, the dimer of N,N-dioleyl-N,N-dimethylammonium chloride (DODAC). All the trans-orientated lipids regardless of their hydrophobic chain lengths (C_{16.1}, C_{18.1} and C_{20.1}) appear to enhance the transfection efficiency compared with their cis-orientated counterparts.

The structures of cationic polymers useful as a delivery vehicle include but are not limited to linear polymers such as chitosan and linear poly(ethyleneimine), branched polymers such as branch poly(ethyleneimine) (PEI), circle-like polymers such as cyclodextrin, network (crosslinked) type polymers such as crosslinked poly(amino acid) (PAA), and dendrimers. Dendrimers consist of a central core molecule, from which several highly branched arms ‘grow’ to form a tree-like structure with a manner of symmetry or asymmetry. Examples of dendrimers include polyamidoamine (PAMAM) and polypropylenimine (PPI) dendrimers.

DOPE and cholesterol are commonly used neutral co-lipids for preparing cationic liposomes. Branched PEI-cholesterol water-soluble lipopolymer conjugates self-assemble into cationic micelles. Pluronic (poloxamer), a non-ionic polymer and SP1017, which is the combination of Pluronics L61 and F127, may also be used.

In one embodiment, PLGA particles are employed to increase the encapsulation frequency although complex formation with PLL may also increase the encapsulation efficiency. Other cationic materials, for example, PEI, DOTMA, DC-Chol, or CTAB, may be used to make nanospheres.

In one embodiment, complexes are embedded in or applied to a material including but not limited to hydrogels of poloxamers, polyacrylamide, poly(2-hydroxyethyl methacrylate), carboxyvinyl-polymers (e.g., Carbopol 934, Goodrich Chemical Co.), cellulose derivatives, e.g., methylcellulose, cellulose acetate and hydroxypropyl cellulose, polyvinyl pyrrolidone or polyvinyl alcohols, or combinations thereof.

In some embodiments, a biocompatible polymeric material is derived from a biodegradable polymeric such as collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N-isopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), or poly(dioxanone) (PPS).

In another embodiment, the biocompatible material includes polyethyleneterephalate, polytetrafluoroethylene, copolymer of polyethylene oxide and polypropylene oxide, a combination of polyglycolic acid and polyhydroxyalkanoate, gelatin, alginate, poly-3-hydroxybutyrate, poly-4-hydroxybutyrate, and polyhydroxyoctanoate, and polyacrylonitrilepolyvinylchlorides.

In one embodiment, the following polymers may be employed, e.g., natural polymers such as starch, chitin, glycosaminoglycans, e.g., hyaluronic acid, dermatan sulfate and chrondrotin sulfate, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol(D,L-lactide-coglycolide), poly(lactic acid colysine) and polycaprolactone.

In one embodiment, the biocompatible material is derived from isolated extracellular matrix (ECM). ECM may be isolated from endothelial layers of various cell populations, tissues and/or organs, e.g., any organ or tissue source including the dermis of the skin, liver, alimentary, respiratory, intestinal, urinary or genital tracks of a warm blooded vertebrate. ECM employed in the invention may be from a combination of sources. Isolated ECM may be prepared as a sheet, in particulate form, gel form and the like.

The biocompatible scaffold polymer may comprise silk, elastin, chitin, chitosan, poly(d-hydroxy acid), poly(anhydrides), or poly(orthoesters). More particularly, the biocompatible polymer may be formed polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy) methane] (PCPM), copolymers of SA, CPP and CPM, poly(amino acids), poly(pseudo amino acids), polyphosphazenes, derivatives of poly[(dichloro)phosphazenes] or poly[(organo) phosphazenes], poly-hydroxybutyric acid, or S-caproic acid, polylactide-co-glycolide, polylactic acid, polyethylene glycol, cellulose, oxidized cellulose, alginate, gelatin or derivatives thereof.

Thus, the polymer may be formed of any of a wide range materials including polymers, including naturally occurring polymers, synthetic polymers, or a combination thereof. In one embodiment, the scaffold comprises biodegradable polymers. In one embodiment, a naturally occurring biodegradable polymer may be modified to provide for a synthetic biodegradable polymer derived from the naturally occurring polymer. In one embodiment, the polymer is a poly(lactic acid) (“PLA”) or poly(lactic-co-glycolic acid) (“PLGA”). In one embodiment, the scaffold polymer includes but is not limited to alginate, chitosan, poly(2-hydroxyethylmethacrylate), xyloglucan, co-polymers of 2-methacryloyloxyethyl phosphorylcholine, poly(vinyl alcohol), silicone, hydrophobic polyesters and hydrophilic polyester, poly(lactide-co-glycolide), N-isoproylacrylamide copolymers, poly(ethylene oxide)/poly(propylene oxide), polylactic acid, poly(orthoesters), polyanhydrides, polyurethanes, copolymers of 2-hydroxyethylmethacrylate and sodium methacrylate, phosphorylcholine, cyclodextrins, polysulfone and polyvinylpyrrolidine, starch, poly-D,L-lactic acid-para-dioxanone-polyethylene glycol block copolymer, polypropylene, poly(ethylene terephthalate), poly(tetrafluoroethylene), poly-epsilon-caprolactone, or crosslinked chitosan hydrogels.

### Pharmaceutical Compositions

The disclosure provides a composition comprising, consisting essentially of, or consisting of the above-described gene transfer vector(s) and a pharmaceutically acceptable (e.g., physiologically acceptable) carrier. In one embodiment, when the composition consists essentially of the gene transfer vector complexed with the cationic polymer and optionally encapsulated in nanoparticles, or recombinant virus, and a pharmaceutically acceptable carrier, additional components can be included that do not materially affect the composition (e.g., adjuvants, buffers, stabilizers, anti-inflammatory agents, solubilizers, preservatives, etc.). In one embodiment, when the composition consists of the gene transfer vector complexed with the cationic polymer and optionally encapsulated in nanoparticles, or recombinant virus, and the pharmaceutically acceptable carrier, the composition does not comprise any additional components. Any suitable carrier can be used within the context of the invention, and such carriers are well known in the art. The choice of carrier will be determined, in part, by the particular site to which the composition may be administered and the particular method used to administer the composition. The composition optionally can be sterile with the exception of, in one embodiment, the gene transfer vector complexed with the cationic polymer and optionally encapsulated in nanoparticles, or recombinant virus, described herein. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, Pa. (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. In one embodiment, the carrier is a buffered saline solution. In one embodiment, the gene transfer vector is administered in a composition formulated to protect the gene transfer vector from damage prior to administration. For example, the composition can be formulated to reduce loss of the gene transfer vector on devices used to prepare, store, or administer the gene transfer vector, such as glassware, syringes, or needles. The composition can be formulated to decrease the light sensitivity and/or temperature sensitivity of the gene transfer vector. To this end, the composition may comprise a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition will extend the shelf life of the gene transfer vector, facilitate administration, and increase the efficiency of the inventive method. Formulations for gene transfer vector containing compositions are further described in, for example, Wright et al., Curr. Opin. Drug Discov. Devel., 6(2): 174-178 (2003) and Wright et al., Molecular Therapy, 12: 171-178 (2005))

The composition also can be formulated to enhance transduction efficiency. In addition, one of ordinary skill in the art will appreciate that the gene transfer vector can be present in a composition with other therapeutic or biologically-active agents. For example, factors that control inflammation, such as ibuprofen or steroids, can be part of the composition to reduce swelling and inflammation associated with in vivo administration of the gene transfer vector. Immune system stimulators or adjuvants, e.g., interleukins, lipopolysaccharide, and double-stranded RNA. Antibiotics, i.e., microbicides and fungicides, can be present to treat existing infection and/or reduce the risk of future infection, such as infection associated with gene transfer procedures.

Injectable depot forms are made by forming microencapsulated matrices of the subject vectors in biodegradable polymers such as polylactide-polyglycolide. Depending on the ratio of vector to polymer, and the nature of the particular polymer employed, the rate of vector release can be controlled. Examples of other biodegradable polymers include poly(orthoesters) and poly(anhydrides). Depot injectable formulations are also prepared by entrapping the vector optionally in a complex with a cationic polymer in liposomes or microemulsions which are compatible with body tissue.

In certain embodiments, a formulation comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration of the inventive gene transfer vector. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

The dose of the gene transfer vector in the composition administered to the mammal will depend on a number of factors, including the size (mass) of the mammal, the extent of any side-effects, the particular route of administration, and the like. In one embodiment, the inventive method comprises administering a “therapeutically effective amount” of the composition comprising the inventive gene transfer vector described herein. A “therapeutically effective amount” refers to an amount effective, at dosages and for periods of time necessary, to achieve a desired therapeutic result. The therapeutically effective amount may vary according to factors such as the extent of the disease or disorder, age, sex, and weight of the individual, and the ability of the gene transfer vector to elicit a desired response in the individual. The dose of gene transfer vector in the composition required to achieve a particular therapeutic effect typically is administered in units of vector genome copies per cell (gc/cell) or vector genome copies/per kilogram of body weight (gc/kg). One of ordinary skill in the art can readily determine an appropriate gene transfer vector dose range to treat a patient having a particular disease or disorder, based on these and other factors that are well known in the art. In one embodiment, the therapeutically effective amount may be between 1×10¹⁰ genome copies to 1×10¹³ genome copies for viruses.

In one embodiment, the composition is administered once to the mammal. It is believed that a single administration of the composition may result in persistent expression in the mammal with minimal side effects. However, in certain cases, it may be appropriate to administer the composition multiple times during a therapeutic period to ensure sufficient exposure of cells to the composition. For example, the composition may be administered to the mammal two or more times (e.g., 2, 3, 4, 5, 6, 6, 8, 9, or 10 or more times) during a therapeutic period.

The present disclosure provides pharmaceutically acceptable compositions which comprise a therapeutically-effective amount of gene transfer vector comprising a nucleic acid sequence as described above.

### Routes of Administration, Dosages and Dosage Forms

Administration of the gene delivery vectors may be continuous or intermittent, depending, for example, upon the recipient's physiological condition, and other factors known to skilled practitioners. The administration of the gene delivery vector(s) may be essentially continuous over a preselected period of time or may be in a series of spaced doses. Both local administration, e.g., intra joint, e.g., intraarticular, intranasal or intrathecal, and systemic administration are contemplated. Any route of administration may be employed, e.g., intravenous, intranasal or intrabronchial, or local administration, e.g., to a joint. In one embodiment, compositions may be delivered to a joint.

One or more suitable unit dosage forms comprising the gene delivery vector(s), anti-fibrotic drug or amobarbital or a derivative thereof, which may optionally be formulated for sustained release, can be administered by a variety of routes including local, e.g., to a joint or intrathecal, oral, or parenteral, including by rectal, buccal, vaginal and sublingual, transdermal, subcutaneous, intravenous, intramuscular, intraperitoneal, intrathoracic, or intrapulmonary routes. The formulations may, where appropriate, be conveniently presented in discrete unit dosage forms and may be prepared by any of the methods well known to pharmacy. Such methods may include the step of bringing into association the vector with liquid carriers, solid matrices, semi-solid carriers, finely divided solid carriers or combinations thereof, and then, if necessary, introducing or shaping the product into the desired delivery system.

The amount of gene delivery vector(s) administered to achieve a particular outcome will vary depending on various factors including, but not limited to, the genes and promoters chosen, the condition, patient specific parameters, e.g., height, weight and age, and whether prevention or treatment, is to be achieved.

Vectors of the invention may conveniently be provided in the form of formulations suitable for administration. A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. By “pharmaceutically acceptable” it is meant a carrier, diluent, excipient, and/or salt that is compatible with the other ingredients of the formulation, and not deleterious to the recipient thereof.

Vectors of the present invention may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Therapeutically useful compositions can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The vectors can be provided in a dosage form containing an amount of a vector effective in one or multiple doses. For viral vectors, the effective dose may be in the range of at least about 107 viral particles, e.g., about 10⁹ viral particles, or about 10¹¹ viral particles. The number of viral particles added may be up to 10¹⁴. For example, when a viral expression vector is employed, about 10⁸ to about 10⁶⁰ gc of viral vector can be administered as nucleic acid or as a packaged virion. In some embodiments, about 10⁹ to about 10¹⁵ copies of viral vector, e.g., per 0.5 to 10 mL, can be administered as nucleic acid or as a packaged virion. Alternatively, the nucleic acids or vectors, can be administered in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results. The amount administered will vary depending on various factors including, but not limited to, the nucleic acid or vector chosen for administration, the disease, the weight, the physical condition, the health, and/or the age of the mammal. Such factors can be readily determined by the clinician employing animal models or other test systems that are available in the art. As noted, the exact dose to be administered is determined by the attending clinician but may be in 1 mL phosphate buffered saline. For delivery of plasmid DNA alone, or plasmid DNA in a complex with other macromolecules, the amount of DNA to be administered will be an amount which results in a beneficial effect to the recipient. For example, from 0.0001 to 1 mg or more, e.g., up to 1 g, in individual or divided doses, e.g., from 0.001 to 0.5 mg, or 0.01 to 0.1 mg, of DNA can be administered.

For example, when a viral expression vector is employed, about 10⁸ to about 10⁶⁰ gc of viral vector can be administered as nucleic acid or as a packaged virion. In some embodiments, about 10⁹ to about 10¹⁵ copies of viral vector, e.g., per 0.5 to 10 mL, can be administered as nucleic acid or as a packaged virion. Alternatively, the nucleic acids or vectors, can be administered in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results.

By way of illustration, liposomes and other lipid-containing gene delivery complexes can be used to deliver one or more transgenes. The principles of the preparation and use of such complexes for gene delivery have been described in the art (see, e.g., Ledley, (1995); Miller et al., (1995); Chonn et al., (1995); Schofield et al., (1995); Brigham et al., (1993)).

Pharmaceutical formulations containing the gene delivery vectors can be prepared by procedures known in the art using well known and readily available ingredients. For example, the agent can be formulated with common excipients, diluents, or carriers, and formed into tablets, capsules, suspensions, powders, and the like. The vectors of the invention can also be formulated as elixirs or solutions appropriate for parenteral administration, for instance, by intramuscular, subcutaneous or intravenous routes.

The pharmaceutical formulations of the vectors can also take the form of an aqueous or anhydrous solution, e.g., a lyophilized formulation, or dispersion, or alternatively the form of an emulsion or suspension.

In one embodiment, the vectors may be formulated for administration, e.g., by injection, for example, bolus injection or continuous infusion via a catheter, and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

For administration to the upper (nasal) or lower respiratory tract by inhalation, the vector is conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the vector may be administered via nose drops, a liquid spray, such as via a plastic bottle atomizer or metered-dose inhaler. Typical of atomizers are the Mistometer (Wintrop) and the Medihaler (Riker).

The local delivery of the vectors can also be by a variety of techniques which administer the vector at or near the site of disease, e.g., using a catheter or needle Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

The formulations and compositions described herein may also contain other ingredients such as antimicrobial agents or preservatives.

### Subjects

The subject may be any animal, including a human and non-human animals. Non-human animals includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses. The subject may also be livestock such as, cattle, swine, sheep, poultry, and horses, or pets, such as dogs and cats.

Subjects include human subjects suffering from or at risk for oxidative damage. The subject is generally diagnosed with the condition of the subject invention by skilled artisans, such as a medical practitioner.

The methods described herein can be employed for subjects of any species, gender, age, ethnic population, or genotype. Accordingly, the term subject includes males and females, and it includes elderly, elderly-to-adult transition age subjects adults, adult-to-pre-adult transition age subjects, and pre-adults, including adolescents, childrents, and infants.

Examples of human ethnic populations include Caucasians, Asians, Hispanics, Africans, African Americans, Native Americans, Semites, and Pacific Islanders. The methods of the invention may be more appropriate for some ethnic populations such as Caucasians, especially northern European populations, as well as Asian populations.

The term subject also includes subjects of any genotype or phenotype as long as they are in need of the invention, as described above. In addition, the subject can have the genotype or phenotype for any hair color, eye color, skin color or any combination thereof.

The term subject includes a subject of any body height, body weight, or any organ or body part size or shape.

### EXEMPLARY EMBODIMENTS

In one embodiment, a composition comprising nanoparticles comprising isolated nucleic acid comprising nucleic acid encoding a mammalian gene, e.g., mammalian lubricin, complexed with a cationic polymer is provided. In one embodiment, the nucleic acid encodes a lubricin having at least 80% amino acid sequence identity to any one of SEQ ID Nos. 1-6. In one embodiment, the nucleic acid encodes a human lubricin. In one embodiment, the cationic polymer comprises a cationic peptide, a linear or branched synthetic polymer, a polysaccharide, a natural polymer or an activated or non-activated dendrimer. In one embodiment, the cationic peptide comprises polylysine or polyornithine. In one embodiment, the cationic linear or branched synthetic polymer comprises polybrene or polyethyleneimine. In one embodiment, the cationic polysaccharide comprises cyclodextrin or chitosan. In one embodiment, the cationic polymer comprises PLGA, PEI, PAMAM or chitosan, or a combination thereof. In one embodiment, the nanoparticles are formed of lactic acid, glycolic acid, caproic acid, or combinations thereof. In one embodiment, the nanoparticles have an average diameter of about 450 nm to about 700 nm. In one embodiment, the nanoparticles have an average diameter of about 550 nm to about 650 nm. In one embodiment, the nanoparticles have an average diameter of about 100 nm to about 150 nm. In one embodiment, the nanoparticles have an average diameter of about 150 nm to about 200 nm. In one embodiment, the nanoparticles comprise a coating. In one embodiment, the coating comprises a polysaccharide or a peptide, e.g., chitosan or PLL. In one embodiment, a virus comprises the nucleic acid encoding lubricin. In one embodiment, the composition further comprises an anti-fibrotic agent, e.g., sulfasalazine, amobarbital, beta-amino propionitrile, cis-hydroxyl proline, blebbistatin or paclitaxel. In one embodiment, the nanoparticles are formed of lipids.

Further provided is a recombinant viral vector comprising nucleic acid encoding a mammalian lubricin. In one embodiment, the nucleic acid encodes a lubricin having at least 80% amino acid sequence identity to any one of SEQ ID Nos. 1-6. In one embodiment, the nucleic acid encodes a human lubricin. In one embodiment, the virus is adenovirus, adeno-associated virus, a herpesvirus, a lentivirus or a retrovirus. In one embodiment, the virus comprises a genome or a capsid of AAV1, AAV2, AAV3, AAV5, AAV6, AAV9 or AAVrh10.

In one embodiment, a method to inhibit or treat injury to cartilage in a mammal is provided. The method includes administering to a joint space in a mammal in need thereof an effective amount of nanoparticles comprising isolated nucleic acid encoding a mammalian lubricin complexed with a cationic polymer or a recombinant viral vector comprising nucleic acid encoding a mammalian lubricin.

In one embodiment, the mammal is a human. In one embodiment, the joint is an ankle, knee or shoulder joint. In one embodiment, the method further comprises administering amobarbital or a derivative thereof. In one embodiment, the method further comprises administering an anti-fibrotic drug. In one embodiment, the nucleic acid comprises a plasmid. In one embodiment, the nucleic acid comprises RNA. In one embodiment, the nucleic acid comprises a constitutive promoter operably linked to the nucleic acid encoding the mammal lubricin.

In one embodiment, a method to treat PTOA in a mammal is provided. The method includes comprising: administering to a joint space in a mammal in need thereof an effective amount of a composition comprising nanoparticles comprising isolated nucleic acid encoding a mammalian lubricin complexed with a cationic polymer or a recombinant viral vector comprising nucleic acid encoding a mammalian lubricin. In one embodiment, the mammal is a human. In one embodiment, the joint is an ankle, knee or shoulder joint. In one embodiment, the method further comprises administering amobarbital or a derivative thereof. In one embodiment, the method further comprises administering an anti-fibrotic drug. In one embodiment, the nucleic acid comprises a plasmid. In one embodiment, the nucleic acid comprises RNA. In one embodiment, the nucleic acid comprises a constitutive promoter operably linked to the nucleic acid encoding the mammal lubricin.

The invention will be described by the following non-limiting examples.

### Example 1

### Rabbit Model of PTOA

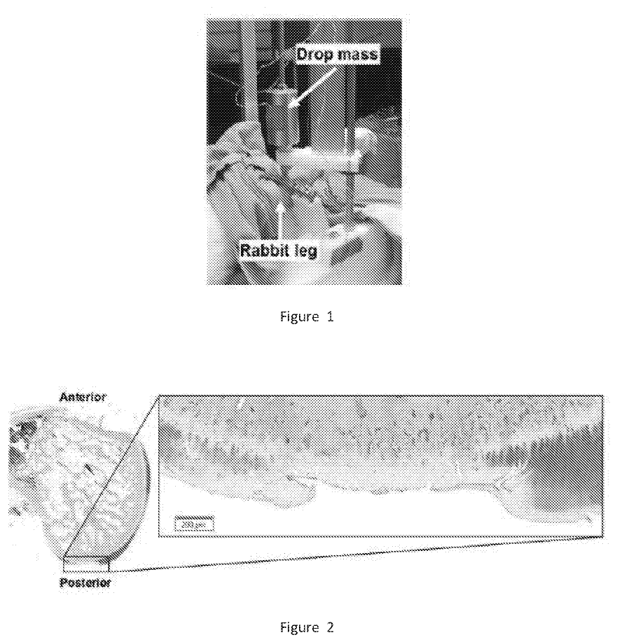

A pilot study was conducted to determine if a high energy impact to cartilage surfaces such as occurs in articular fractures is sufficient to induce post-traumatic osteoarthritis (PTOA). New Zealand White rabbits were subjected to an impaction insult to the left knee. In the surgery, each animal was positioned prone, and the posterior aspect of the medial femoral condyle was approached through a posterior arthrotomy. The experimental knee was secured on a custom fixation platform, and a 7 mm diameter flat metal platen was placed on the medial femoral surface (FIG. 1). A force pulse was then delivered by dropping a 1.55-kg mass onto the platen. The peak compressive impact force during impaction was measured using an accelerometer mounted in the drop mass. No impaction force was delivered for the remaining 8 (sham surgery control) animals. Localized osteoarticular injury led to joint-wide histological changes defined as the (ratio of the defect cross-sectional area to the estimated normal cartilage cross-sectional area in a 4 mm antero-posterior span in the central part of habitual weight-bearing region). Changes in the medial femoral surfaces were measured in terms of structural damage, cartilage thickness, cell density, and proteoglycan (PG) staining.

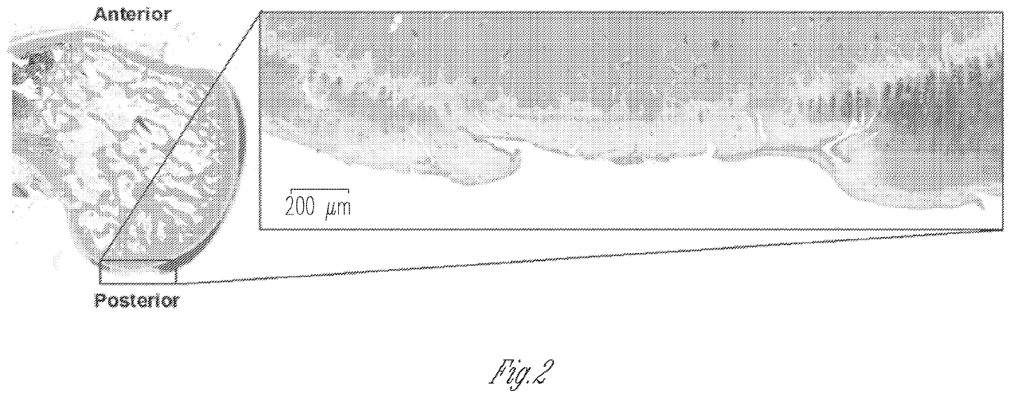

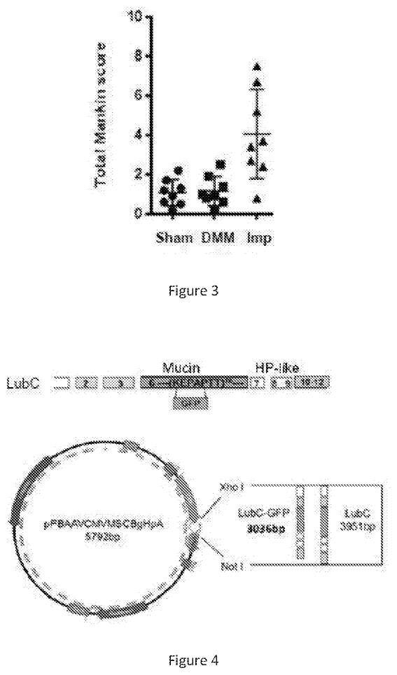

All impacted joints had a cartilage defect on the posterior aspect of the medial femoral surface. The size of defect (mean: 17.8±13.4%) exhibited significant positive correlation (r=0.60) with peak impact force. At the time of euthanasia at 8 or 16 weeks post-surgery, the experimental knees were subjected to histo-morphological evaluation (FIG. 2). Histological Mankin scores were significantly higher than in the sham surgery group (p<0.01) on the medial femoral surfaces (FIG. 3).

### Lubricant Loss and Supplementation

Lubricin, otherwise known as proteoglycan 4 (PRG4), is a heavily glycosylated protein that acts as a cartilage lubricant by virtue of its water-binding capacity and c-terminal hemopexin-like domain, that allows it to bind with high affinity to the cartilage extracellular matrix, forming a protective coating (Jay & Waller, 2014; Jones et al., 2009).

Various PRG4 isoforms are expressed in multiple tissues, but the bulk of the PRG4 found in joint fluid and cartilage surfaces is secreted by synoviocytes and chondrocytes. Joint injuries and inflammation increase PRG4 degradation and suppress de novo synthesis, leading to PRG4 deficiency and increased friction between cartilage surfaces (Jones et al., 2009; Elsaid et al., 2007; Elsaid et al., 2005; Jay et al., 2007). Repeated intra-articular injections of recombinant PRG4 over 1-2 weeks has been shown to mitigate PTOA in animal models, indicating that PRG4 loss is pathogenic (Flannery, 2010; Flannery et al., 2009).

### Lubricin Delivery Via AAV-Mediated Gene Therapy In Vivo

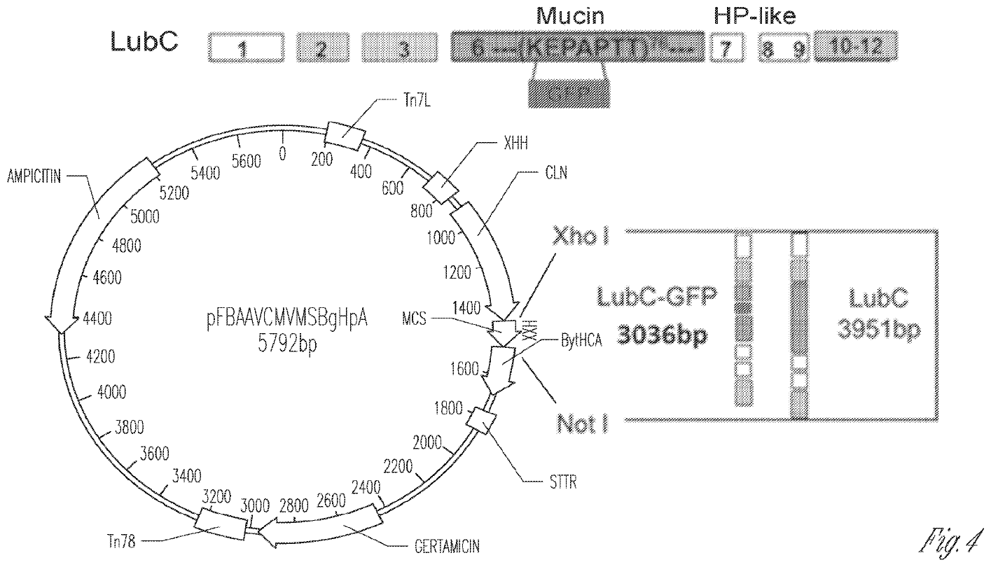

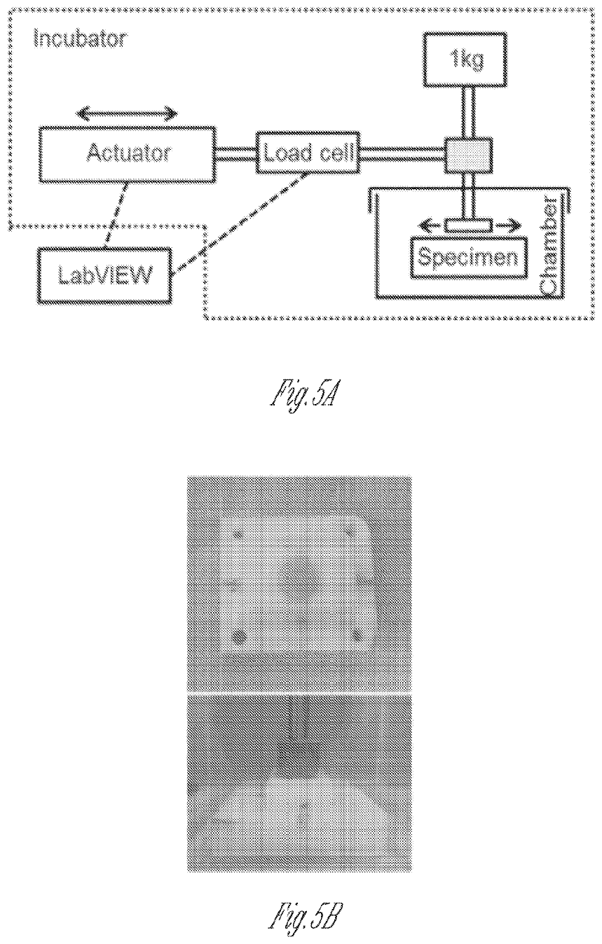

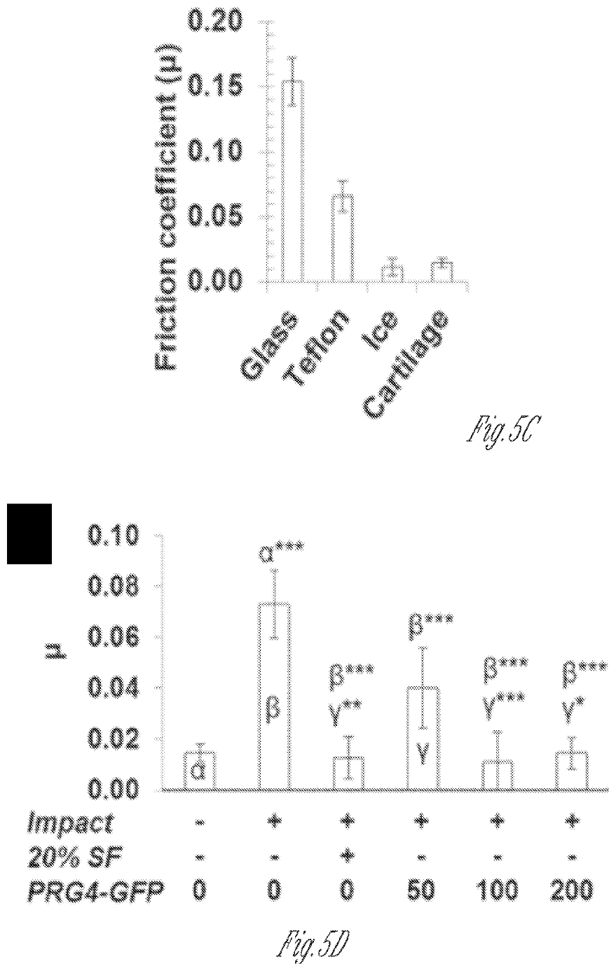

Favorable results from studies of PRG4 therapy in animal models have not led to translation to people due in part to difficulties in sustaining therapeutic levels of the recombinant peptide, which can only be achieved with multiple intra-articular injections; hence, the need for approaches that extend the benefits of a single treatment. By ensuring a long-term, steady supply of transgenic protein from cells within the joint, intra-articular PRG4 gene therapy offers a means to address this challenge. This strategy was implemented in rabbit and mouse PTOA models and it was found that transgene expression can be sustained for up to 8 weeks after a single treatment. In preparation, a version of the PRG4 gene was cloned in an AAV-compatible plasmid in which expression is driven by the constitutively active cytomegalovirus (CMV) promoter (FIG. 4). For tracking purposes, a short sequence in the PRG4 mucin domain was replaced with a sequence encoding green fluorescent protein (GFP). The fusion protein was fully functional as a cartilage lubricant (FIG. 5) was chondroprotective in a an in vitro model (FIG. 6) and in a rabbit ACLT model (FIG. 7). PRG4 expression in synovial tissue was confirmed by rtPCR at 8 weeks post-op (FIG. 8).

### Non-Viral Gene Therapy

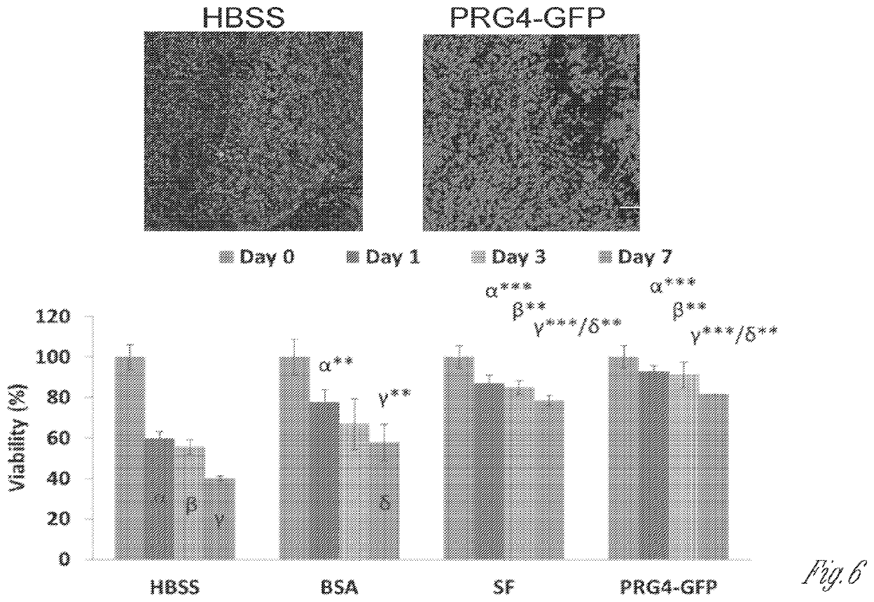

A mouse model of PTOA induced by destabilization of the medial meniscus is well established in our laboratory. To explore the therapeutic potential of anti-inflammatory microRNAs (miR) injured mouse stifle joints were injected with a 5 μg of a plasmid in which miR-17 expression is driven by the U6 promoter (PMIS-17). Mice were euthanized one week after surgery and DNA injection. RNA was extracted from whole joints and the polymerase chain reaction (PCR) was performed using miR-17-specific primers (FIG. 9).

### Sustained Plasmid DNA Delivery

The success with virus-free gene delivery in mice provided an opportunity to enhance the response further by delivering DNA in a sustained-release polymer formulation. A promising polymer for providing sustained plasmid DNA (pDNA) delivery is poly (lactic-co-glycolic acid) (PLGA), which has an FDA approved track record as a vehicle for drug, protein, and pDNA delivery (Abbas et al., 2008). Therapeutic agents loaded in PLGA particles have shown efficient endo-lysosomal escape and sustained intracytoplasmic delivery. Biodegradable PLGA particles are biocompatible and have the capacity to protect pDNA from nuclease degradation and increase pDNA stability (Zhang et al., 2008). The inclusion of excipients such as cationic polymers can protect pDNA loaded into the PLGA particles and improve loading rates (D'Mello et al., 2017). These pDNA loaded PLGA particles exhibited low toxicity and efficient transgene expression profiles. The first step in preparing PLGA pDNA particles is selecting the appropriate molecular weight of PLGA and ratios of lactide and glycolide exhibit to tailor degradation rates for specific applications.

### Objectives

Post-traumatic lubricin deficiency likely promotes PTOA independently from the pathogenic processes targeted by amobarbital. The overall objective is to determine if enhancing PRG4/lubricin expression in injured joints by gene therapy augments the chondroprotective effects of amobarbital. Thus, a non-viral method of gene delivery suitable for use in humans was tested.

A slow release formulation of PRG4-encoding plasmid DNA is likely more efficient and less toxic than the same dose given as a bolus.

Non-viral PRG4 gene delivery may be as effective as viral delivery in mitigating PTOA in a rabbit model.

Restoring cartilage lubrication likely enhances the chondroprotective effects of amobarbital in fractured joints, particularly when residual incongruity is present.

When paired with amobarbital, lubricin gene therapy may improve results at 12 months post-fracture in the porcine IAF model. Lubricin gene delivery methods may be tested in a rabbit PTOA model in prior to testing in the porcine IAF model.

Chondrocyte and synoviocyte cultures are used to identify formulations for in vivo gene transfer, formulations that have enhanced transfection efficiency and reduced toxicity. Various excipients are screened and tested in sustained release versus bolus formulations (Table 1).

TABLE 1

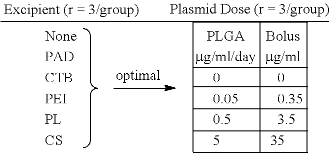

Several cationic excipients are tested to determine if they improve transfection rates and reduce toxicity compared with un-complexed DNA. Excipients include polyamidoamine dendrimers (PAD), cetyl trimethylammonium bromide (CTB), polyethyleneimine (PEI), polylysine (PL), and chitosan CS (Abbas et al., 2008).

Sustained release of optimal DNA-excipient complexes via encapsulation in PLGA microparticles is compared to bolus dosing with respect to toxicity and transfection rate.

### Compare Expression Levels and Therapeutic Effects of PRG4-GFP Delivered by AAV Versus Non-Viral Delivery in a Rabbit Model of PTOA

A head to-head comparison between adeno-associated virus (AAV) and a non-viral method is performed in a rabbit impact model of PTOA (Table 2). AAV-like therapeutic effects may be achieved via non-viral plasmid transfection.

| Treatment | n |
|---|---|
| AAV-Control | 6 |
| AAV-PRG4 | 6 |
| Plasmid Control | 6 |
| Plasmid PRG4 | 6 |
| Total : | 24 |

Table 2

Determine if PRG4 Gene Therapy Forestalls PTOA Progression in Anatomically-Reduced and Malreduced IAFs Treated with Amobarbital

Insufficient cartilage lubrication may promote PTOA in fractured joints independently of the cellular processes targeted by amobarbital; thus, enhancing lubricin expression may augment the therapeutic effects of amobarbital in the Yucatan minipig IAF model. These effects may be especially evident in mal-reduced joints, as contact stresses and friction are exaggerated by incongruity. Treatment groups include amobarbital alone PRG4 alone, and a combination of the two (Table 3).

| | Reduction | | |
|---|---|---|---|
| Treatment | AR (Aim 2) | MR (Aim 3) | 25 |
| Amo | 3 | 6 | |
| PRG4 | 6 | 6 | |
| Amo + PRG4 | 6 | 6 | |

Iota! :33
Table 3 The same treatments are tested in
joints with anatomically reduced (AR) and
mal-reduced (MR) joints.30

### Strata

### Identify an Optimal Formulation for PRG4-GFP Plasmid Delivery

Although transfecting cells in vivo with a single injection of un-complexed plasmid DNA at concentrations ranging from 1 to 5 μg/mL was shown to be successful, these concentrations are close to levels that have proven cytotoxic in some settings. Moreover, it is likely that much of the free DNA injected in joints is cleared or degraded before plasmids can be taken up by cells. Delivering lower doses for longer periods of time may result in similar or higher transfection rates, while lessening the chances of toxicity.

### Study Design

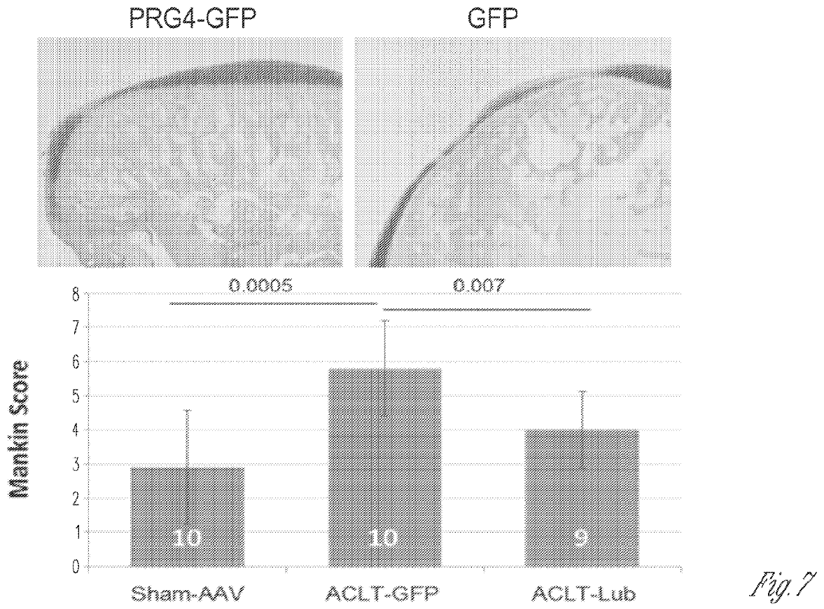

The toxicity and transfection efficiency of various excipient-DNA-PLGA particles are assessed in monolayer cultures of synovial fibroblasts and chondrocytes (FIGS. 3-10). A low toxicity (<10% mortality) formulation that results in the highest level of transgene expression will be carried forward to animal studies.

### Methods

Cell culture: Synovial fibroblasts and chondrocytes are harvested from rabbit stifle joints obtained post-euthanasia from other IUCUC-approved studies conducted at the University of Iowa. Cell will be seeded in 96-well plates (30,000/well), and in 24-well plates (100,000/well). Fibroblasts are cultured in Dulbecco's Modified Eagle Medium (DMEM) supplemented with 10% fetal bovine serum, 1% penicillin/streptomycin, and 0.1% amphotericin B. Chondrocytes are cultured in DMEM/Ham's F12 (1:1) with the same supplements. Cultures are incubated in a humidified chamber at 37° C. in a 5% O₂ and 5% CO₂ atmosphere.

Cytotoxicity: The toxicity of the various cationic excipients is tested by MTS assay (Cell Titer 96 AQueous One Solution cell proliferation assay, Promega Corporation). The medium is replaced after 24 hours and DNA-excipient complexes are then added in concentrations ranging from 0 to 5 μg/ml. After 48 hours, the cells ar washed and the MTS assay is performed according to the manufacturer's instructions. Relative levels of soluble formazan formed after 4 hours of incubation aree measured with a SpectraMax Plus384 Microplate Reader (Molecular Devices, Sunnyvale, Calif., USA) at an absorbance of 490 nm. Percent cell viability is calculated as the ratio of the sample absorbance to control.

PLGA/DNA formulation: The double-emulsion solvent-evaporation technique is one of the most common methods used for encapsulating pDNA. The method involves the use of three phases: 1) an inner water phase containing the DNA to be incorporated, 2) an intermediate organic phase consisting of a polymer/organic solvent solution, and 3) an outer water phase containing an emulsifying agent. The organic solvent is removed and the particles isolated and dried. PLGA particle size can be controlled by varying the stirring rate and the surfactant concentration. A series of methods for optimizing the loading of pDNA into PLGA particles has been described (Intra & Salem, 2011). The double emulsion method tends to produce solid particles with very small pDNA containing “pockets” well dispersed throughout the matrix.

Physico-chemical characterization of the DNA loaded PLGA Particles Scanning electron microscopy (SEM) is employed to characterize surface morphology and shape of the particles. Surface charge and particle size analysis including polydispersity and particle size distribution are carried out using a Zetasizer Nano ZS as described previously (Abbas et al., 2008). Loading and release of pDNA from the PLGA particles are carried out using a UV Spectrophotometer as described previously (Zhang et al., 2008). The targets are in a size range of 500 to 600 nm (with an average 0.2 polydispersition), a net positive surface charge with values ranging from +2.40 to +9.88 mV, and loading of 10 μg pDNA/mg particles.

The target size range is 500 to 600 nm with a mean 0.2 polydispersity, a positive zetapotential value and loading of 10 μg pDNA/mg particles. Excipient-pDNA-PLGA particles likely have a net positive surface charge with values ranging from +2.40 to +9.88 mV and a target loading of 10 μg pDNA/mg particles.

Gene expression analysis: PCR is used to measure transgene expression. Total RNA is extracted from cells using an RNeasy Mini Kit (Qiagen) following the protocol provided by the manufacturer. Equal amounts of RNA from each sample are converted to cDNA. cDNA is mixed in 96-well Optical Reaction Plates (Applied Biosystems), and PCR reactions are performed in an Applied Biosystems 7300 Real-Time PCR System. Steady-state mRNA levels are normalized to 18s rRNA and calculated relative to untreated controls by the relative quantitation method (^{2ΔΔ}CT).

PRG4-GFP expression: The PRG4-GFP clone encodes a signal peptide sequence for secretion; thus, expression levels can be measured in growth medium from transfected cell cultures. A GFP ELISA kit is used to measure PRG4-GFP in media samples according to the suppliers protocol (Cell Biolabs, catalog #AKR-121). Medium (0.1 ml) i assayed and the data are normalized to DNA.

DNA assay: Cells layers are collected by trypsinization and centrifugation. Cell pellets are resuspended in 1 mL of RNAse/DNAse free water and then sonicated for 30 seconds. DNA concentrations are measured using Hoechst 33342 fluorescence measured at 460 nm with excitation at 360 nm.

### Compare Expression Levels and Therapeutic Effects of PRG4-GFP Delivered by AAV Versus Non-Viral

Delivery in a Rabbit Model of PTOA Joints are injected with AAV or optimized PRG4-GFP plasmid DNA formulation immediately after impact to the femoral condyle. Rabbits are euthanized 8 weeks post-injury to assess the effects of treatment on PTOA progression and chondrocyte viability in and around impact sites.

### Study Design

Equal numbers of male and female adult New Zealand White rabbits are entered in the study. The joints are injected intra-operatively at the time of impact injury with AAV or a non-viral formulation bearing the GFP (control) or the PRG4-GFP genes (Table 2). Weight-bearing is allowed immediately. A survival time of 8 weeks is sufficient to develop measurable PTOA progression.

### Methods

The rabbit impact model described above is implemented to study the effects of lubricin gene therapy on the progression of cartilage degeneration at 8 weeks after a 2J impact.

Transgene expression is assessed by GFP ELISA of synovial extracts.

Chondrocyte viability in and around impact sites is assessed by confocal microscopy (see FIG. 6).

Safranin-O histology remains the gold standard for measuring the severity of PTOA and is the main outcome measure for evaluating cartilage degeneration and subchondral bone changes. An objective image analysis algorithm is used to derive Mankin scores based on structural damage, proteoglycan density, cellularity, and tidemark integrity (FIG. 11) (Arunakul et al., 2013; Coleman et al., 2017; Goetz et al., 2017; Goetz et al., 2015; Vaseenon et al., 2011).

Determine if PRG4 Gene Therapy Forestalls PTOA Progression in Anatomically-Reduced and Malreduced IAFs Treated with Amobarbital

Hock joint fractures in Yucatan minipigs are anatomically reduced or mal-reduced with a 2 mm step-off by internal fixation. Joints are treated immediately after fracture with PRG4 gene therapy alone, or in combination with amobarbital. These agents are delivered via intra-articular injection of 0.5 ml of a hyaluronan-based hydrogel as described previously (Coleman et al., 2017).

### Study Design

Fractured joints are treated as shown in Table 3. All animals are euthanized at 12 months post-fracture to evaluate the progression of PTOA.

### Methods

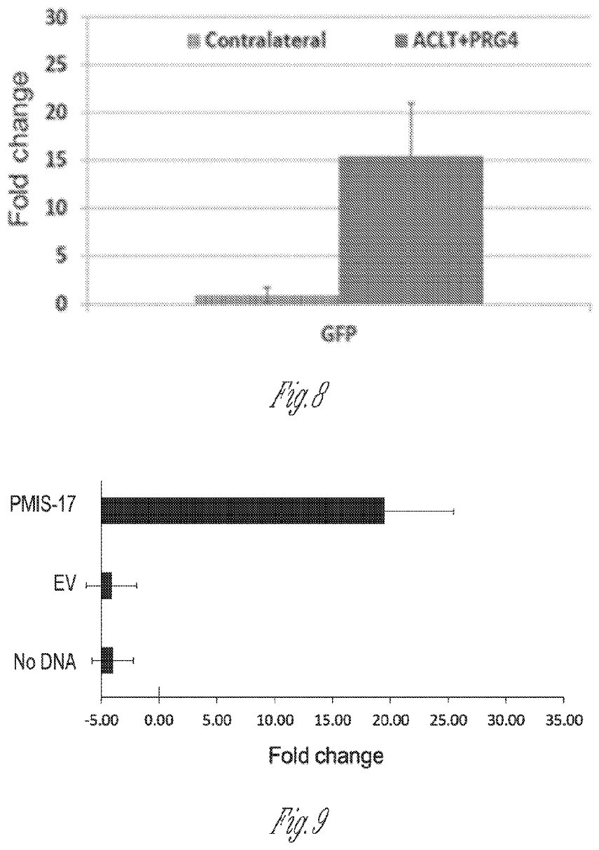

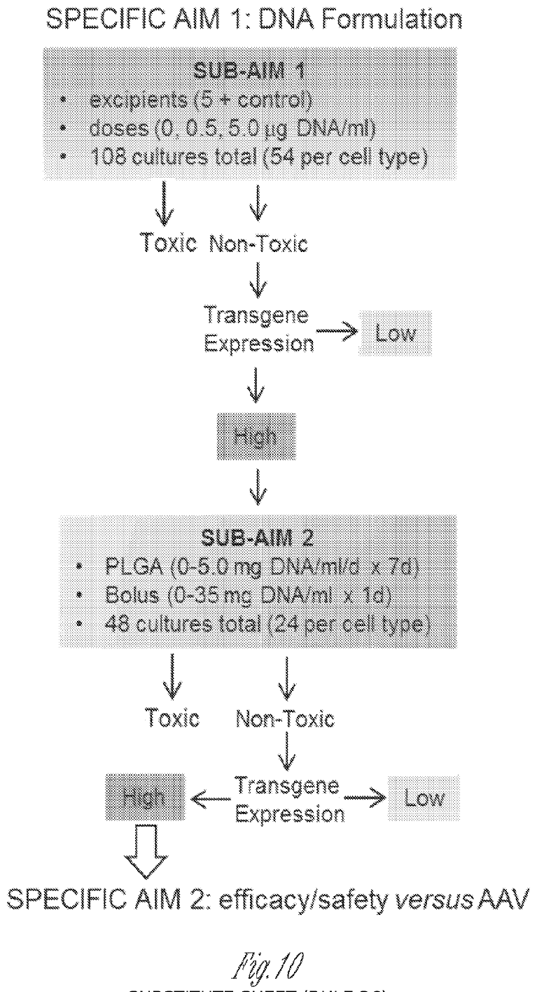

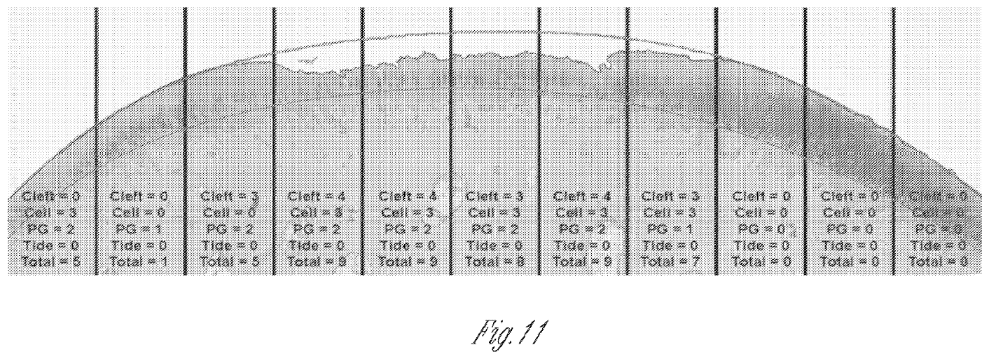

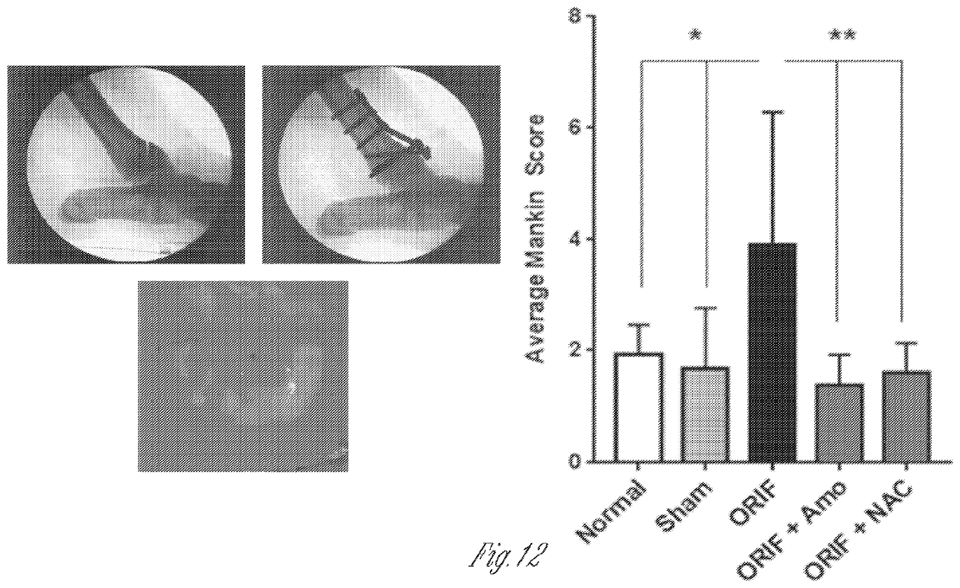

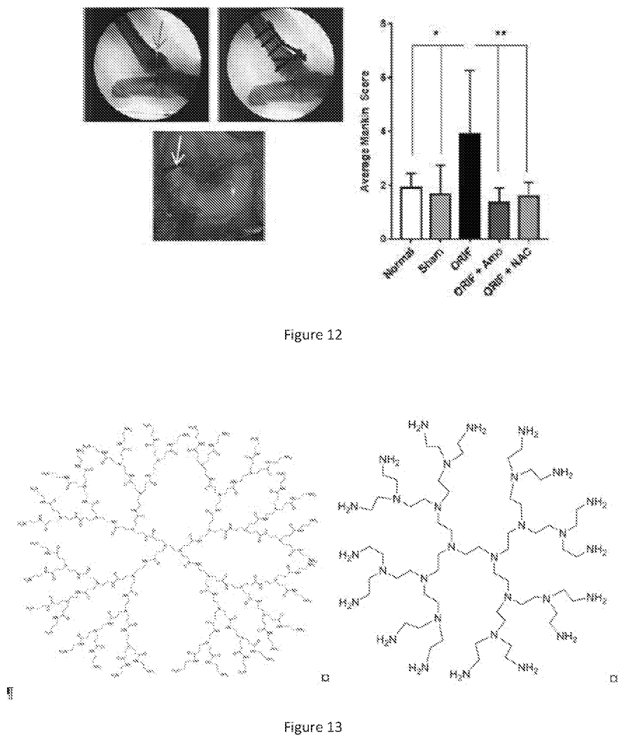

Yucatan Minipig Intra-articular Fracture Model. The Yucatan minipig model was developed to create an IAF of a reproducible severity without arthrotomy (Goetz et al., 2015). Under general anesthesia, an antero-medial approach to the hock joint, which is analogous to the human ankle joint, is made without disrupting the joint capsule. Under fluoroscopic visualization, a custom impaction tripod is rigidly affixed to the talus from the caudal (posterior/inferior) side of the leg. A stress-rising saw cut is made through the anterior tibial cortex, stopping 1-2 mm proximal to the subchondral bone. Two drill holes are made proximal to the saw cut into which a plate with a ball-joint is press fit. This ball joint interfaces with the instrumented sled on our impaction pendulum to rigidly support the tibia. The pendulum delivers a 40 J impact to the impaction tripod, driving the talus against the distal tibia causing a fracture of the distal tibia that is guided by the stress-rising saw cut. The result is a fracture fragment that consists of the anterior third of the distal tibial articular surface which is fixed using standard open reduction and internal fixation (ORIF) techniques (FIG. 12). Hock fractures in the Yucatan minipig leads reliably to moderate PTOA at 6 months-post op, with additional progression to 12 months (Coleman et al., 2017; Goetz et al., 2015). Intra-operative amobarbital treatment by joint injection with 2.5 mM amobarbital in anatomically-reduced joints was highly effective in preventing changes at 6 months (FIG. 12). but was less effective at 12 months (Coleman et al., 2017). In this study it is determined if lubricin gene therapy is an effective adjunct to amobarbital at the later time point, with or without residual incongruity; thus, all animals in this study survive to 12 months post-fracture. As in precedent studies, equal numbers of boars and sows will be included. Although amobarbital has already been tested in reduced fractures, a small group is included in this study as a check on reproducibility (Table 3).

GFP ELISA is used to quantify the GFP and PRG4-GFP content of synovial fluids and extracts of synovium samples.

Activity restriction/lameness is scored on a 0-4 scale on a monthly basis by qualified veterinary personnel.

Safranin-O histology with automated image analysis is used to score cartilage degeneration and subchondral bone changes. H&E histology will be used to assess synovitis (monocyte infiltration, fibrosis).

### Statistics

Two-way analysis of variance (ANOVA) is applied to determine the influence of concentration and formulation on the outcome measures. PLGA and bolus delivery are both tested in replicate cultures derived from the same rabbit; thus, paired t-tests may reveal important formulation effects within dose levels that might not be captured by ANOVA.

Mankin scores are categorical in nature; thus, treatment effects on this outcome measure are assessed using non-parametric Mann-Whitney U tests. Based on past experience impact injury will double mean Mankin scores versus sham controls, an effect that lubricin therapy reduced by an average of 30% In that study it was found that 6 rabbits/per group gave sufficient power to detect injury and treatment effects (p<0.05, power=0.85). Correlations between transgene expression levels and Mankin score will be evaluated by Pearson product moment analysis.

The main goal is to assess the effects of the two treatments alone and in combination on Mankin scores. These data are analyzed using the Mann-Whitney U test. The program also outputs continuous measurements on safranin-O staining density and other OA-related changes. These data and gene expression data are assessed using one-way ANOVA with Kruskal-Wallace post-hoc tests. Prior experiments indicate that 6 animals/per group is sufficient to detect significant effects of fracture and treatment on Mankin scores with p<0.05 and with a power of >0.8.

### Impact

It has been shown in an advanced animal model that, when delivered soon after injury, amobarbital blocks acute cellular responses that initiate PTOA. Although these effects are extremely promising, long-term patho-mechanical factors may eventually lead to PTOA even in joints treated with amobarbital, a challenge that PRG4 gene therapy has the potential to address. Testing a thoroughly vetted gene therapy approach in a clinically-realistic setting provides definitive data on the translational significance of this concept, which could revolutionize the treatment of PTOA.

### Example 2

When a bone fracture occurs across a joint this is called an intra-articular fracture which can lead to debilitating side effects for the patient such as post-traumatic osteoarthritis (PTOA). PTOA is a progressively degenerative and irreversible condition that affects over six million individuals in the US. Though there are multiple strategies that can be used to mitigate PTOA, decreasing the damaging shear stress at the articular surface by improving the lubrication of the joint is of interest. Lubricin, also known as proteoglycan 4 (PRG4), is a protein which serves as a lubricant in joints by forming a protective coating along the cartilage extracellular matrix. The majority of PRG4 found in the joint fluid is secreted by synoviocyte and chondrocyte cells. The inflammation that results from injury to joints leads to increased PRG4 degradation and downregulates de novo synthesis. This suppression of PRG4 synthesis means that there is increased friction between cartilage surfaces leading to damaging shear stress.

Poly(lactic-co-glycolic) acid (PLGA) is a copolymer that is degradable and FDA approved for use in drug delivery systems. In one embodiment, a PLGA-based formulation for PRG4-GFP pDNA delivery effectively and efficiently transfects synoviocyte and chondrocyte cells with low toxicity. This is accomplished by first synthesizing nanoplexes of pDNA and a cationic agent, then encapsulating these nanoplexes into PLGA nanoparticles. The cationic agents that were tested for the formulation were polyamidoamine (PAMAM) dendrimer generation 5 and polyethyleneimine (PEI) (FIG. 13).

### Methods:

Plasmid Amplification and Purification. Two types of plasmids were used in this study both of which encoded for GFP and either did or did not contain the PRG4 gene. The pDNA was purified and concentration determined prior to use.

Preparation and characterization of pDNA/cationic excipient nanoplexes. Nanoplexes were prepared by ionic interaction between the positively charged amine groups on the cationic agent and the negatively charged phosphate moieties of the pDNA. Formulations were made at varying ratios of amine to phosphate groups (N/P ratio) with the amount of DNA kept constant at 50 μg. Varying concentrations of aqueous solutions of either PAMAM or PEI were added to the pDNA solution, immediately vortexed for 30 seconds, then left to incubate at room temperature for 30 minutes. The hydrodynamic diameter, polydispersity index (PDI) and zeta potential of each N/P ratio of nanoplex was measured using Zetasizer. The optimal N/P ratio for complete pDNA complexation was determined using a gel retardation assay.

Preparation of PLGA nanoparticles. PLGA nanoparticles were prepared using the double emulsion, solvent evaporation technique and a probe sonicator. Briefly, 200 mg of PLGA was dissolved in 1.5 mL of dichloromethane (DCM) and to this 100 μL of the desired N/P ratio of nanoplexes was added and sonicated. This emulsion was then added to 8 mL of 1% (w/v) poly(vinyl)alcohol (PVA) and sonicated. Then the second emulsion was rapidly added to 22 mL of 1% PVA and left stirring for two hours to allow for evaporation of the DCM. The nanoparticles were collected using centrifugation and pellet was washed with nanopure water and lyophilized until a dry powder. In order to determine the pDNA loading of the particles, the supernatant was incubated with 1% (w/v) heparin sodium salt to release the DNA from the nanoplex and the PicoGreen assay used. The morphology and size of the PLGA nanoparticles was determined using scanning electron microscopy.

### Results

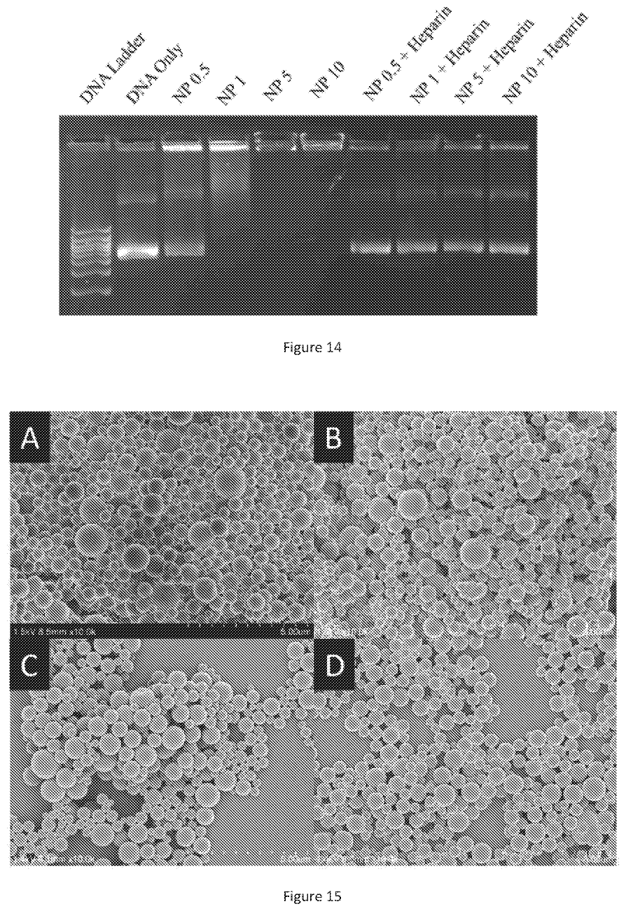

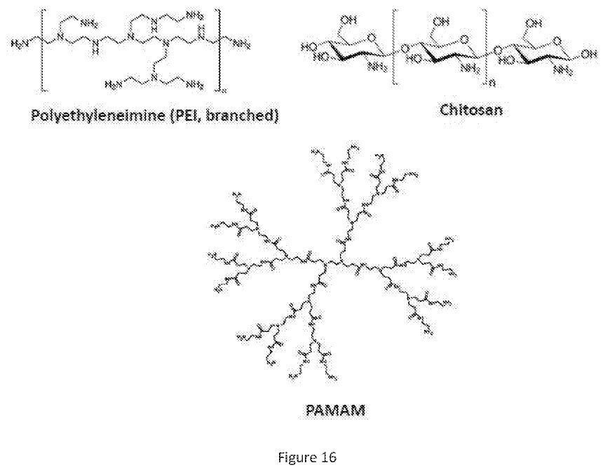

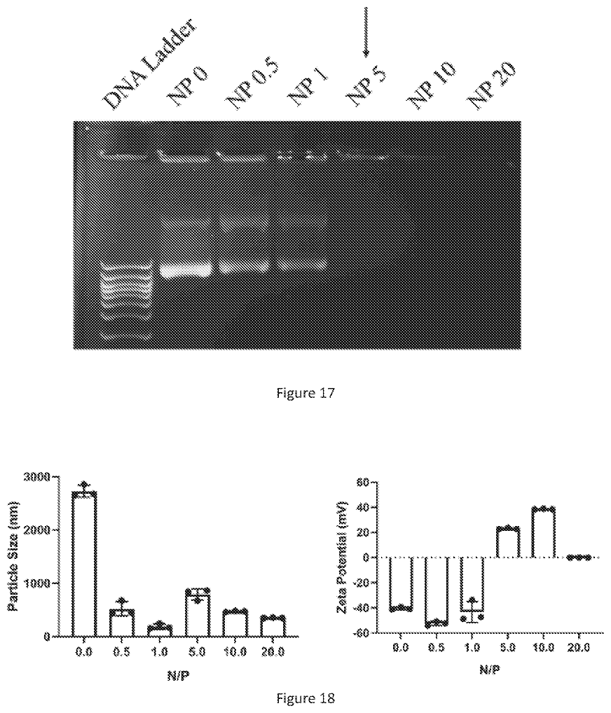

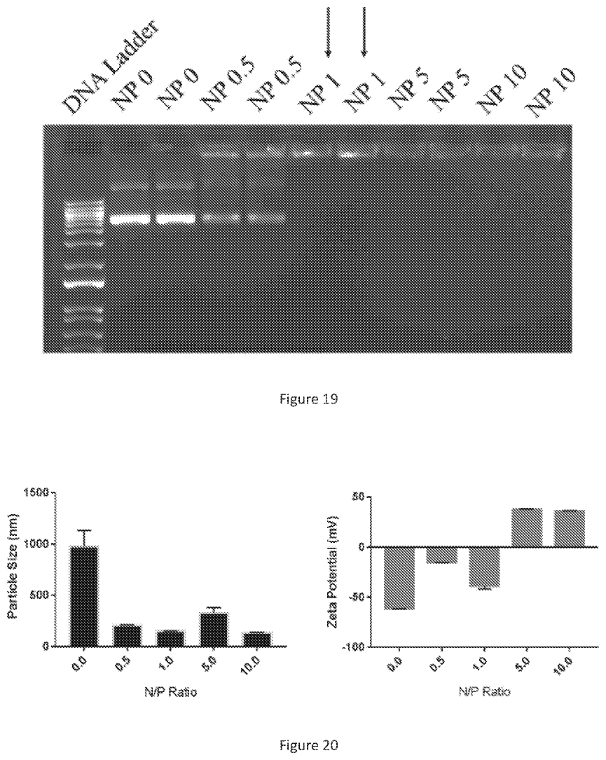

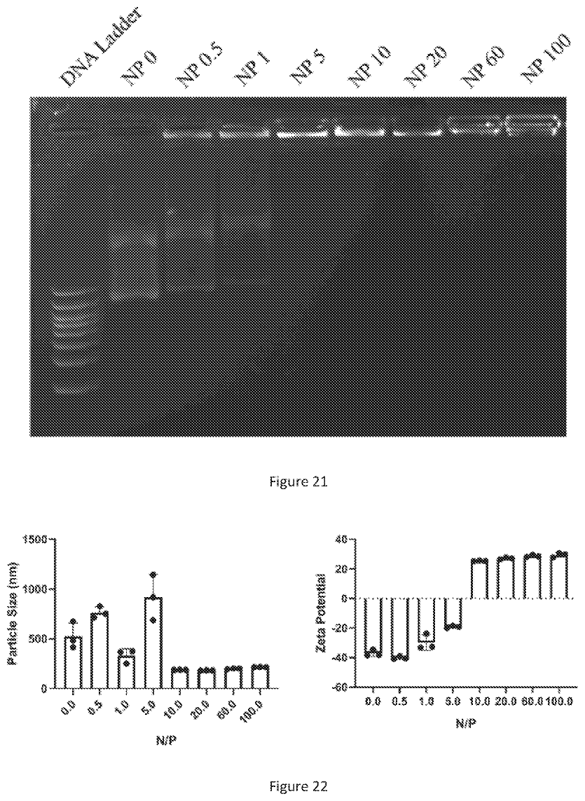

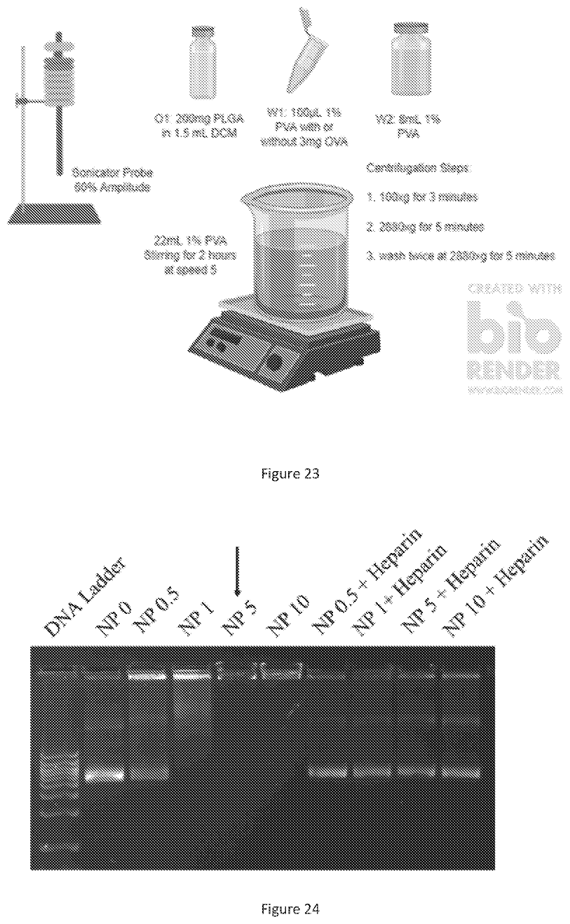

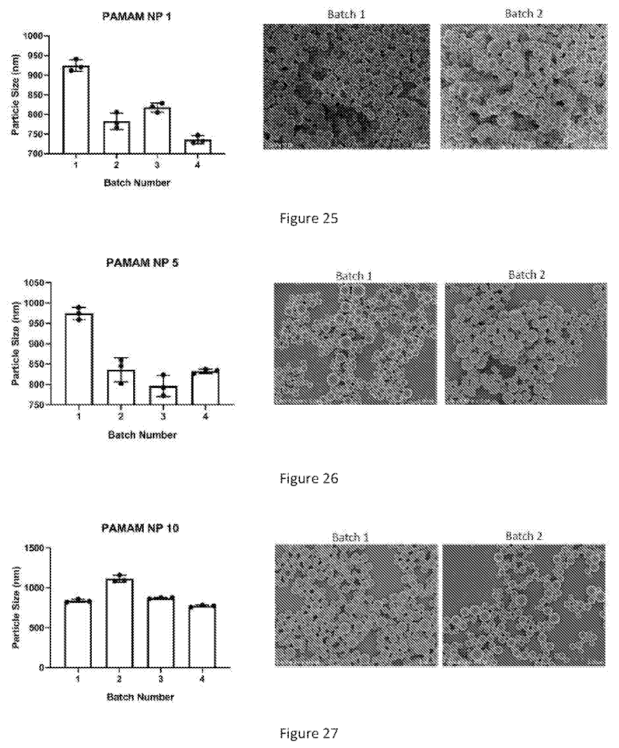

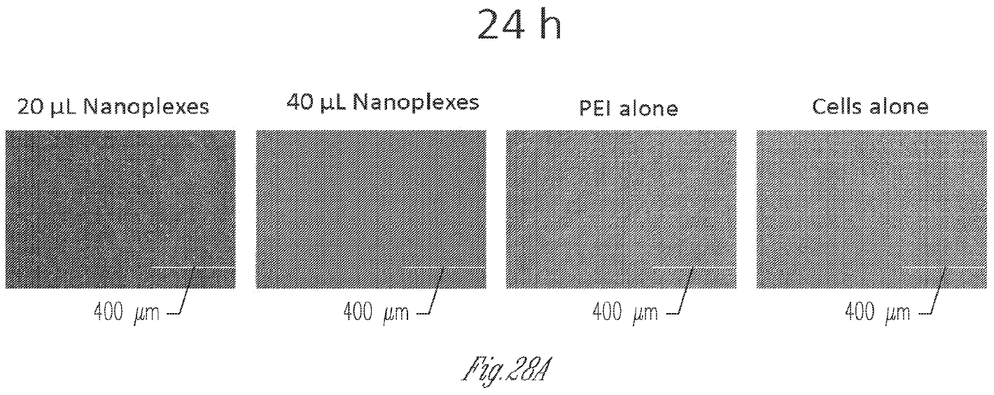

Using the successfully amplified and purified pDNA, nanoplexes were made using both PEI and PAMAM cationic agents. Gel retardation assays (FIG. 14) confirm strong complexation at the higher N/P ratios and that heparin at 5% w/v can competitively bind with the cationic agent and release the pDNA from the complex (FIG. 14). The double emulsion, solvent evaporation technique was used to synthesis PLGA nanoparticles encapsulating the nanoplexes of the desired N/P ratios. The resulting particles had an average size of ˜600 nm and had a smooth and spherical morphology (FIG. 15). The encapsulation efficiency of the particles was between 70 to 83%.

### Conclusion

In conclusion, PTOA is a debilitating condition that results in the downregulation of the lubricin gene and increased shear stress within the joint. In this project we were able to take pDNA encoding for lubricin and successfully complex it with the cationic polymers PAMAM or PEI to synthesize nanoplexes. These nanoplexes were then encapsulated into the degradable PLGA nanoparticles. This novel delivery system for the lubricin gene has the potential to be a local and sustained release therapeutic to upregulate the production of lubricin in damaged joints and thus mitigate the negative side effects of PTOA.

### Example 3

The intent of this study was to follow typical clinical practice in which treatment is initiated when patients present with symptoms of joint stiffness, after fibrosis is already established. It was hypothesized that local treatment with one or more of the drugs described above would alleviate existing joint stiffness in an established rabbit model, in which fibrosis of the stifle (knee) joint is achieved by inducing hemarthrosis, followed by rigid fixation in flexion (Hildebrad et al., 2004; Nesternko et al., 2009) To maintain therapeutic drug levels for up to 3 weeks without resorting to multiple joint injections, fibrosed joints were treated once by implantation of drugs encapsulated in poly[lactic-co-glycolic] acid (PLGA) pellets, a well-established prolonged-release delivery vehicle (Leelakanok et al., 2018). Joint stiffness evaluated by flexion/extension tests served as the primary measure of treatment effects. Secondary effects on ACL stability, and synovial intimal thickness were also assessed. Fibroblasts isolated from joint capsules were subjected to in vitro tests to quantify the effects of immobilization and in vivo treatment on contractile activity and α-SMA expression.

### Methods

### Animal Care and Surgery

### Ethical Statement

All procedures performed in the study were pre-approved by the Institutional Animal Care and Use Committee (IACUC) at the University of Iowa, which is Public Health Service (PHS) Animal Welfare Assurance compliant (D16-00009, A3021-01), accredited by the Association for the Assessment and Accreditation of Laboratory Animal Care, International (No. 000833) and registered as a United States Department of Agriculture research facility (No. 42-R-0004).

### Study Design

The minimal number of animals was used to assess the specific aims of this study. Eight-rabbits were included in each of the treatment groups. Sample size analysis indicated that 8 animals per group should be sufficient to detect treatment effects on knee stiffness of 50% or more. Non-immobilized, untreated (normal) joints were included to provide normative baselines for joint mobility and histologic outcomes. The study included this normal group, as well as an immobilized group implanted with blank pellets (control), and immobilized groups treated with BAPN-, CHP-, or SSZ-loaded pellets. Joints were immobilized for 8 weeks to establish arthrofibrosis/capsule contracture as previously described (Nesterenko et al., 2009). Kirschner-wires (K-wires) were then removed and the various pellets implanted in the joint. After another 8 weeks the animals were euthanized, and the joints immediately tested for stiffness in extension and anterior-posterior (A-P) laxity. Capsule tissues were harvested for histology to evaluate in situ α-SMA expression and intima thickness, and for in vitro quantitation of cellular contractile activity and α-SMA expression.

### Experimental Animals

Specific pathogen-free male New Zealand White rabbits (n=80) weighing 3.0-4.5 kg (age range 7-9 months) obtained from Covance were acclimated for at least 10 days prior to surgery. Each rabbit had a thorough physical examination prior to surgery.

### Housing and Husbandry

Animals were housed individually in standard cages (5.43 ft squared, floor space) located in IACUC approved animal housing space under controlled room conditions. Light/dark cycle was 12/12, temperature and humidity ranged from 18.9-20° C., and 30-50% respectively. Rabbits had free access to water 24/7 and fed Invigo Global High Fiber Rabbit Diet (#2031).

### Experimental Procedures

Joint immobilization surgery: Rabbits were sedated with ketamine (9.184-45.870 mg/kg)/acepromazine (0.055-0.275 mg/kg)/xylazine (0.275-1.375 mg/kg) intramuscularly followed by general anesthesia via isoflurane (1-5% in 02). A 2 mm-diameter burr was used to create bleeding above the medial and lateral collateral ligaments. A 5 mm skin incision was made mid-diaphysis on the medial tibia and a 1.6 mm-diameter Kirschner wire (K-wire) was inserted in a craniolateral direction and tunneled subcutaneously to the lateral side of the femur, where a short incision was made in the skin and then in the underlying lateral fascia. Muscle bellies were separated and retracted, and then the K-wire was bent and wrapped around the cranial portion of the femur. The wire was pulled taught and secured to the tibia by bending the wire flat, thus pulling the tibia toward the femur and fixing the stifle in flexion. Local anesthetic (0.5% Bupivacaine, total dose not to exceed 8 mg/kg) was applied to the surgery site, and then fascial and skin incisions were closed in a routine manner with resorbable sutures. Sustained Release Buprenorphine (SR-Buprenorphine): A dose of 0.1-0.3 mg/kg, was given subcutaneously pre-operative (op) and a second dose was given 72 hours post-op. Sustained Release Meloxicam (SR-Meloxicam): one dose (0.8 mg/kg) was given pre-op subcutaneous, and a second dose was given 72 hours post-op.

Fixation removal surgery and pellet placement: Rabbits were sedated and anesthetized as stated above. The K-wire was removed via two small incisions, local anesthetic applied to sites, and tissues dosed in routine surgical manner. Pellets were secured to the patellar tendon (distal to the patella) with 6.0 resorbable sutures as shown and arthrotomies were closed (Figure S-1). Analgesics were administered as stated above. All surgeries were performed between 08:00 and 16:00 in an IACUC approved surgical suite under aseptic conditions.

Post-operative care: The animals were monitored at least twice a day for the duration of the study for attitude, pain score, surgical site, eating/drinking habits, and urination/defecation habits. Surgical sites were examined daily for signs of infection or inflammation (swelling, discharge, redness, heat, etc.). Surgical sites were also monitored for self-trauma (rabbit pulling sutures out, excessive licking, etc.). If these conditions were noted, an E-collar was placed on the animal to prevent the animal from inflicting further damage. Rabbits in the immobilization group were monitored for abnormal/excessive movement in the fixed limb (left hind limb), or abnormal use/movement of contralateral limb (right hind limb) indicating that immobilization had failed, or a fracture or other injury of a limb was present.

### Mechanical Testing

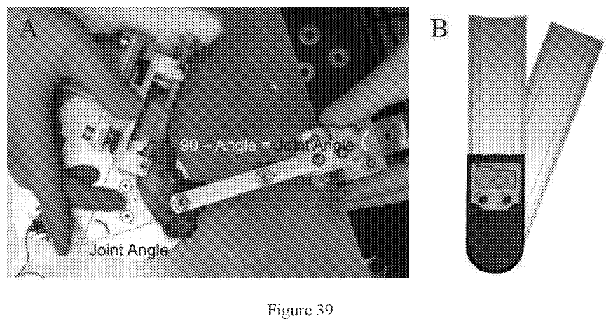

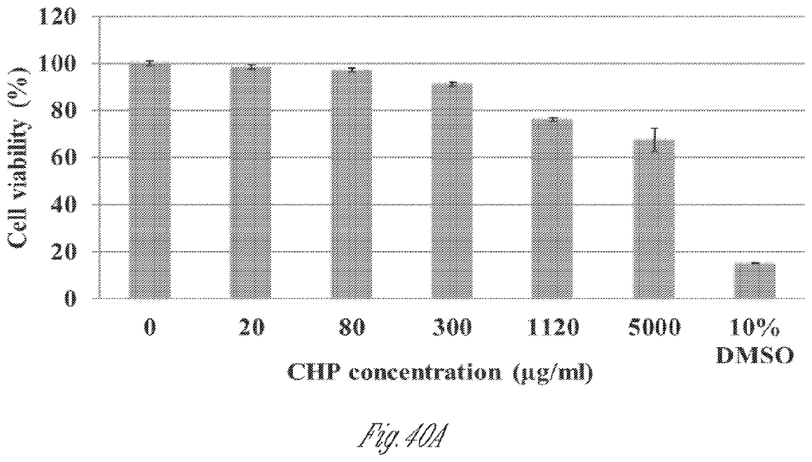

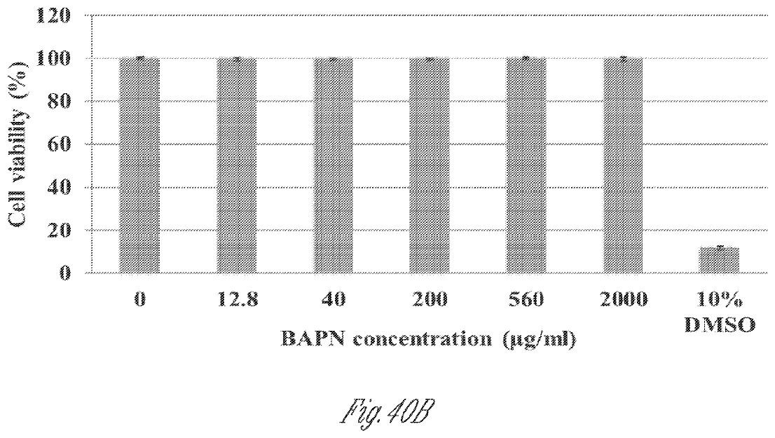

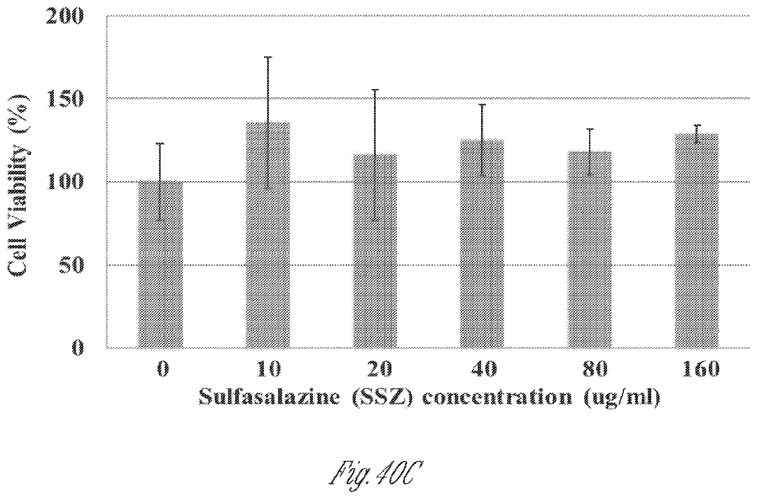

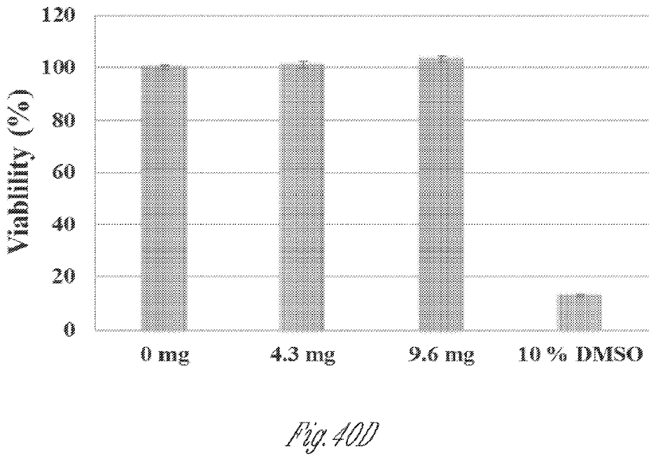

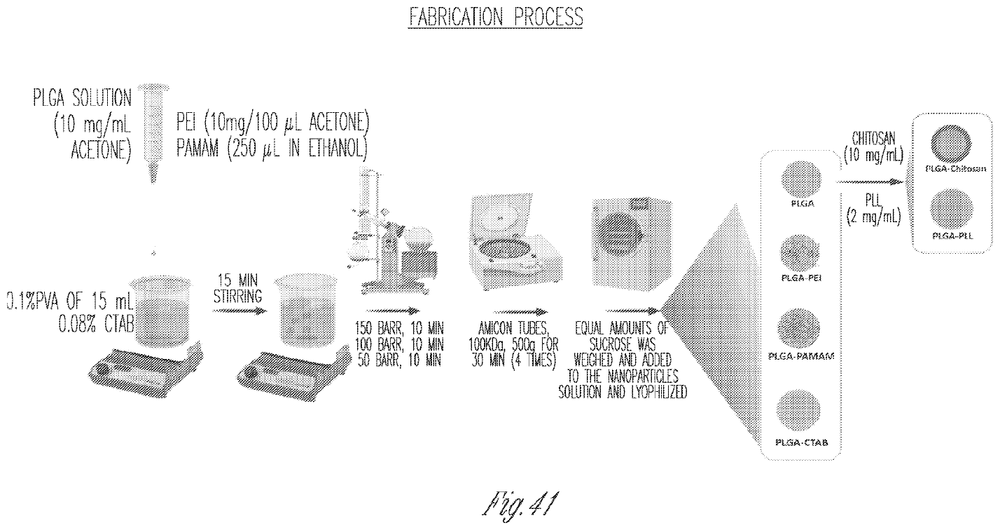

Flexion-extension testing of normal and fibrotic joints was performed immediately after euthanasia, when limbs were disarticulated at the hip, stripped of skin and muscle, and fastened securely to a custom testing device equipped with a stepper motor and a 10 N load cell and controlled by a computer with a custom LabVIEW (National Instruments)-based program (FIG. 38) (Hildebrand et al., 2004; Nesterenko et al., 2009). Starting at 10° of extension, the limbs were driven to 170° of extension, and then returned to the starting position at a rotational velocity of 5°/s while continuously recording load and position. The starting extension angle was measured using a digital protractor (Barry Wixey Development) (FIG. 39). The peak torque at maximum extension (N-m) and dissipated energy (mJ) were derived as measures of joint stiffness. The increased hysteresis in immobilized joints arises from posterior capsule stiffening, which restricts extension, but not flexion. Measurements were based on the first flexion-extension cycle, as stiffness declined significantly with subsequent cycling, consistent with stretching or failure of capsule tissues during the first cycle.

After flexion-extension testing, a custom loading fixture was used to assess sagittal-plane laxity (drawer test), which is related to cruciate ligament integrity (Tochigi et al., 2011). Femurs were secured vertically by means of half-pins, and the tibia secured with transverse pins attached to a horizontal leg holder, with the knee held flexed at 90°. The tibial leg holder was attached to a LabVIEW-controlled stepper motor-driven actuator with force feedback via an inline load cell. The tibial leg holder with affixed limb performed a movement of ±3 mm A-P from the central anatomical position at 1.0 mm/s unless the load limit of ±75 N was reached. Force and displacement data were continuously recorded through the sequence of tests, which consisted of three cycles to adjust leg placement for neutral starting position, followed by three cycles of data collection. The anterior stability of the knee was quantified as the slope of the anterior loading curve outside of the neutral zone, a quantity termed anterior drawer stiffness

### Pellet Fabrication

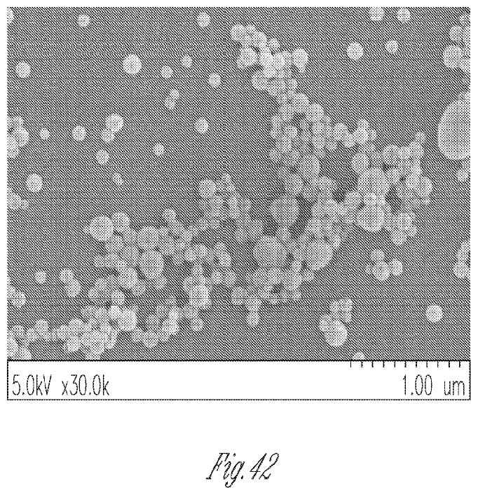

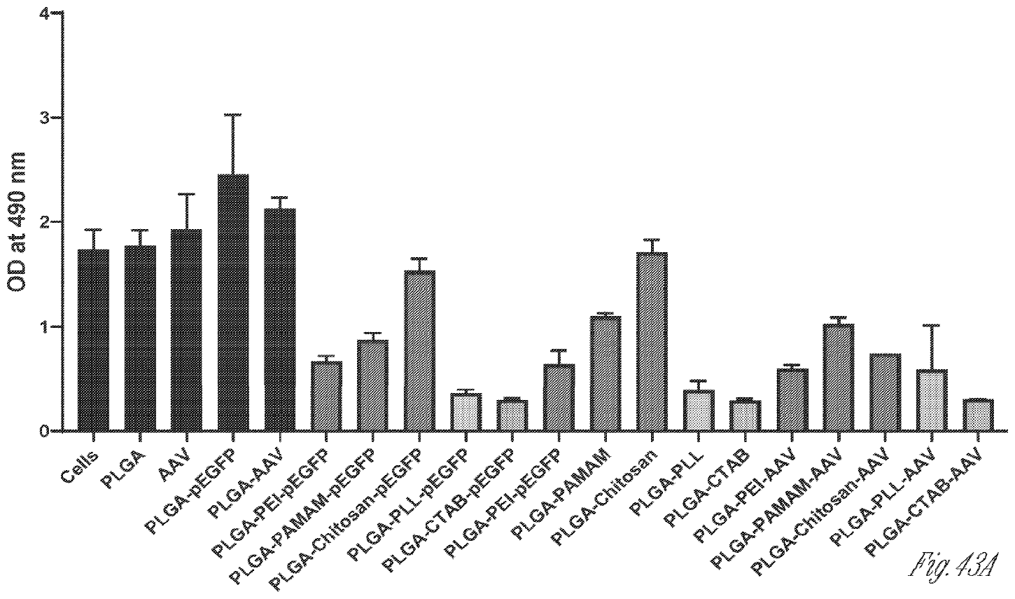

Drug-loaded PLGA pellets were made by the hot-melt extrusion method (Leelakanok et al., 2008; Danyuo et al., 2017). The goal was to load the maximum amount of drug possible in the pellets, which varies depending on the physical and chemical properties of the drugs and their behavior during the hot melt extrusion process. The maximum doses carried in PLGA pellets were 120±3.6 μg SSZ/mg pellet, 126±5 μg BAPN/mg pellet, and 250±3.2 μg CHP/mg pellet. In vitro tests confirmed the drugs and pellets were not cytotoxic (FIG. 40).

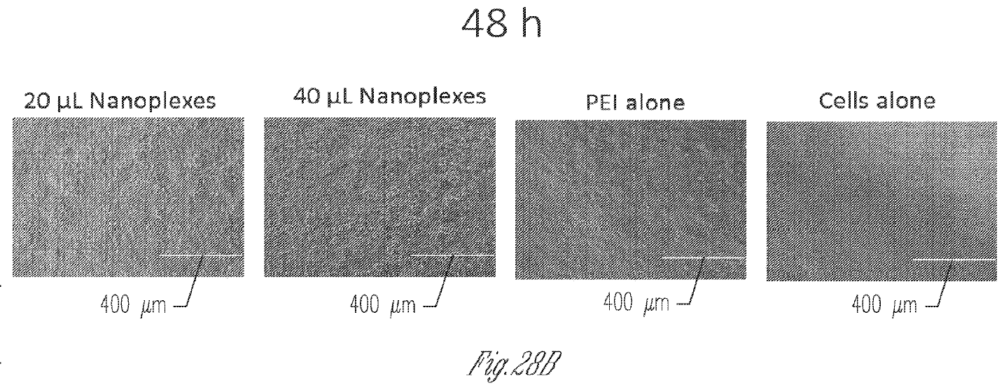

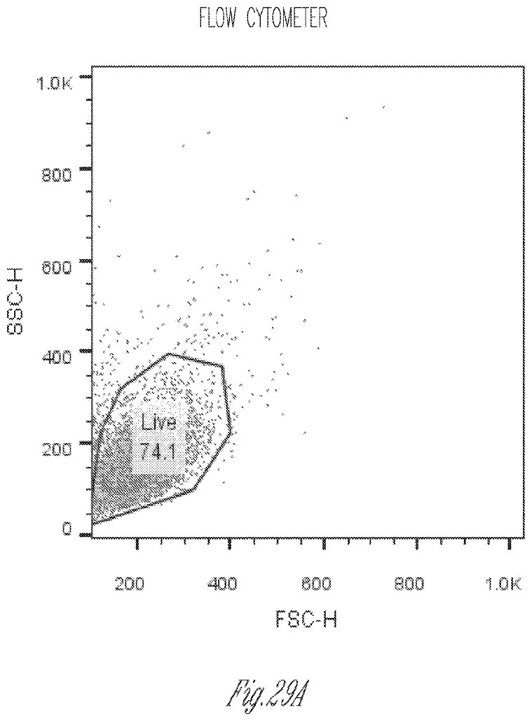

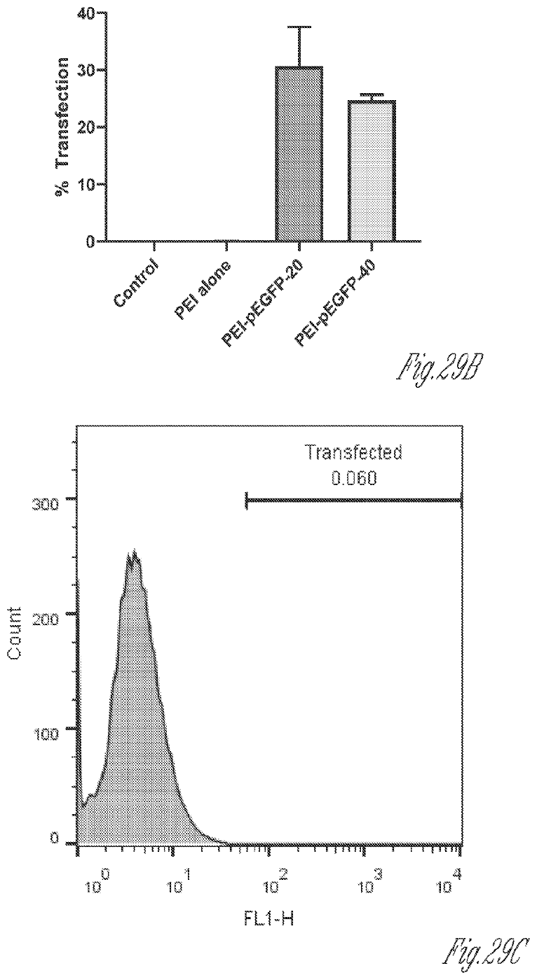

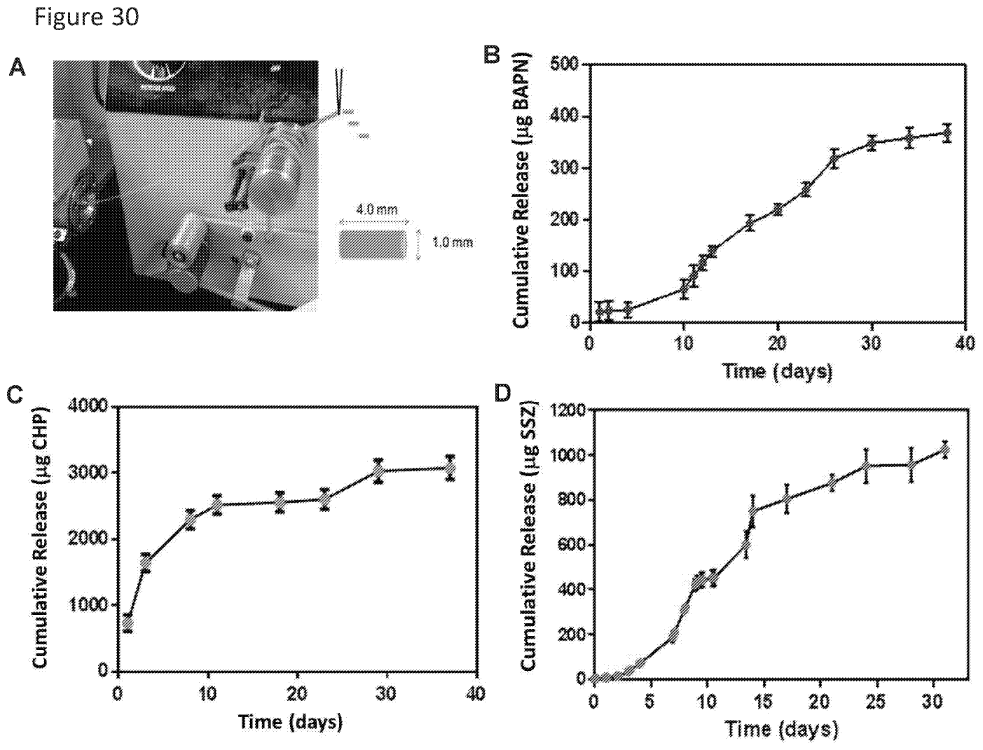

In addition to the drugs, pellets contained 10% polyethylene glycol (PEG 400), and 60% PLGA (50:50, inherent viscosity 0.38 dL/g). PEG 400 was added as plasticizer to lower extrusion temperatures, protecting the drugs from thermal degradation. Before weighing, PLGA was granulated with a mortar and pestle. A small amount (about 8 mg) of a red food dye (FD&C red #40) was mixed with drug to ensure even distribution in the polymer matrix during the extrusion process. Granulated PLGA was triturated in geometric proportions with active ingredient/dye using a mortar and pestle. Then, PEG was added to the mixture of PLGA-active ingredient/dye and incorporated with a mortar and pestle. The mixtures were hot melt extruded by a twin-screw extruder (Minilab II, Thermo Fisher Scientific) with a die diameter of 1 mm. Extrudates were cut into 4 mm-long sections (pellets) (FIG. 30A).

To determine the drug content of the pellets, the pellets were individually dissolved in chloroform (500 μL) and then mixed with 1 mL water to let the active ingredient migrate to the aqueous phase for at least 30 minutes. Drug elution profiles were characterized in vitro prior to implantation. Elution was measured by incubation of the pellets in phosphate buffered saline (PBS) at 37° C. in a shaking incubator set to 300 rpm (FIGS. 30B, C, and D) (Leelakanok et al., 2018; Danyuo et al., 2017). At each time point, PBS was removed from the pellets and replaced with fresh PBS. The amount of BAPN released at each time point was determined using a Fluorescamine assay followed by fluorescent spectroscopy (excitation wavelength: 390 nm, emission wavelength: 475 nm). The amount of CHP released at each time point was determined by hydroxyproline assay with spectrophotometric detection at 560 nm. The amount of SSZ released at each time point was determined using UV spectroscopy at 359 nm.

### Capsule Histology

Portions of the posterior capsule were fixed in 10% neutral buffered formalin, and then processed for paraffin histology. Five μm-thick sections were stained with Harris' hematoxylin and eosin (H&E), and α-SMA immunohistochemistry (IHC) was performed with an anti-α-SMA mouse monoclonal antibody (Abcam) and a biotinylated goat anti-mouse immunoglobulin G (IgG) secondary antibody (Vector). Stained slides were imaged using an Olympus VS110 Virtual Microscope System, and intimal thickness was quantified at 5 random areas in the synovial intima using ImageJ.

### Joint Capsule Fibroblast (JCF) Culture

Capsule tissues were harvested and transferred to a T-75 flask with 15 mL of Dulbecco's Modified Eagle Medium/Ham's Formula-12 Nutrient Mixture (DMEM/F-12) media containing 10% fetal bovine serum (FBS), 1% Penn Strep, and 1% Amphotericin B, and incubated at 37° C. in a 5% O₂, 5% CO₂ atmosphere for about 5 days to allow cells to migrate out of the tissue and adhere to the plate. The JCFs were cultured until confluent (about 2 weeks) before analysis.

### Collagen Gel Compaction Assay

Collagen gel compaction assays were used to determine the contractile activity of JCFs isolated from variously treated rabbits (Yuda & McCulloch, 2018). Collagen gels were produced by combining 10×PBS containing phenol red, 1N NaOH, and rat tail type I collagen (2 mg/mL) (Invitrogen), and JCFs to give a final cell concentration of 1×10⁶ cells/mL. Five hundred μL of the collagen gel mixture was added to each well in a 24-well plate and allowed to polymerize for 30 minutes in an incubator. After 30 minutes, 500 μL of DMEM containing 10% FBS and 50 μg/mL ascorbic acid was added to each well. The plates were then incubated for 24 hours to allow the cells to generate a state of isometric tension. After 24 hours, the medium was replaced with 500 μL of fresh DMEM containing 10% FBS and ascorbic acid. After 48 hours, the gels were detached from the walls of the wells using a sterile spatula to allow the cells to freely compact the gels. Images were acquired over the next 48 hours, and the changes in gel cross sectional area were measured with ImageJ.

DNA assays were used to control for well-well variations in cell numbers that could affect the rate and extent of gel compaction. After 48 hours of compaction, the medium was aspirated and the gels were washed with PBS thrice. Five hundred μL of 0.25 mg/mL collagenase was added and the plates incubated at 37° C. overnight. Cell pellets were collected by centrifuging at 500 g for 10 minutes. The supernatants were discarded and the pellets frozen at −80° C. overnight. After thawing at room temperature, the pellets were suspended in 1.0 mL of RNAse/DNAse-free water and then sonicated for 30 minutes. DNA concentrations were measured using 0.2 μg/mL of Hoechst 33342 in 10×TNE buffer (100 mM Tris, 2.0 M NaCl, and 10 mM EDTA at pH 7.4) with excitation and emission wavelengths at 360 nm and 460 nm respectively with calf thymus DNA as a standard. A calibration curve was generated to relate DNA concentration to the number of JCFs in the cultures.

### α-SMA Immunostaining

JCFs were seeded at a density of 10,000 cells per mm² on UV-sterilized glass coverslips. After 48 hours, the medium was discarded and the samples were washed with Dulbecco's PBS (DPBS). The samples were fixed in 4% paraformaldehyde for 1 hour and then rinsed thrice with DPBS. The cells were then permeabilized with 0.5% Triton X for 5 minutes at room temperature and blocked with 10% goat serum for 30 minutes. The samples were then incubated with an anti-α-SMA mouse monoclonal antibody (Abcam) at a 1:100 dilution in DPBS overnight at 4° C. After washing in DPBS, the cells were blocked with goat serum for 30 minutes at 37° C. and then incubated with a fluorophore-conjugated goat anti-mouse IgG secondary antibody (488 excitation) (Life Technologies) at a 1:100 dilution for 30 minutes at 37° C. Cells were then washed thrice with DPBS, incubated with phalloidin at a 1:100 dilution for 30 minutes at 37° C., and then mounted with 4′,6-diamidino-2-phenylindole (DAPI)-containing mounting medium. Stained sections were imaged using an Olympus FV1000 confocal microscope. Percent positive α-SMA (immunostained/DAPI-stained×100) was evaluated in at least 4 microscope fields using ImageJ.

### Statistics

The experimental unit in this study was the individual animal. The statistical significance of differences between the control group and all other groups was assessed using GraphPad® software. Normally-distributed data (mechanical tests and compaction) were analyzed by one-way analysis of variance (ANOVA) with the Tukey post-hoc test and plotted as individual values with group means and standard deviations indicated. The power of the tests at p=0.05 is indicated in figure legends. Non-normally distributed data (% α-SMA positive) were evaluated by Kruskal-Wallace one-way ANOVA on ranks with Dunn's post-hoc test. P values of less than 0.05 were considered significant. The data were graphed in box and whisker plots showing individual values, the median (horizontal line), 25^{th}, 75^{th} percentiles (lower and upper box limits), and maximum and minimum values (vertical error bars). Linear regression analysis was used to evaluate the strength of correlations among the various outcome measures.

### Results

### Drug Elution Profile

Drug elution profiles were characterized in vitro (FIGS. 30B, C, and D). PLGA pellets released drugs in a sustained and controlled manner over 2-3 weeks. There was a higher burst release for CHP-loaded pellets than for SSZ- or BAPN-loaded pellets.

### Macroscopic and Microscopic Observation of Joint Fibrosis

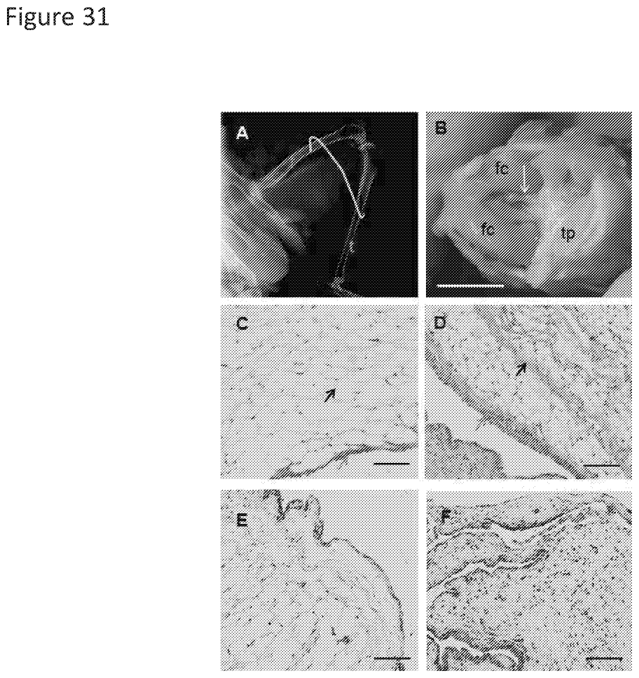

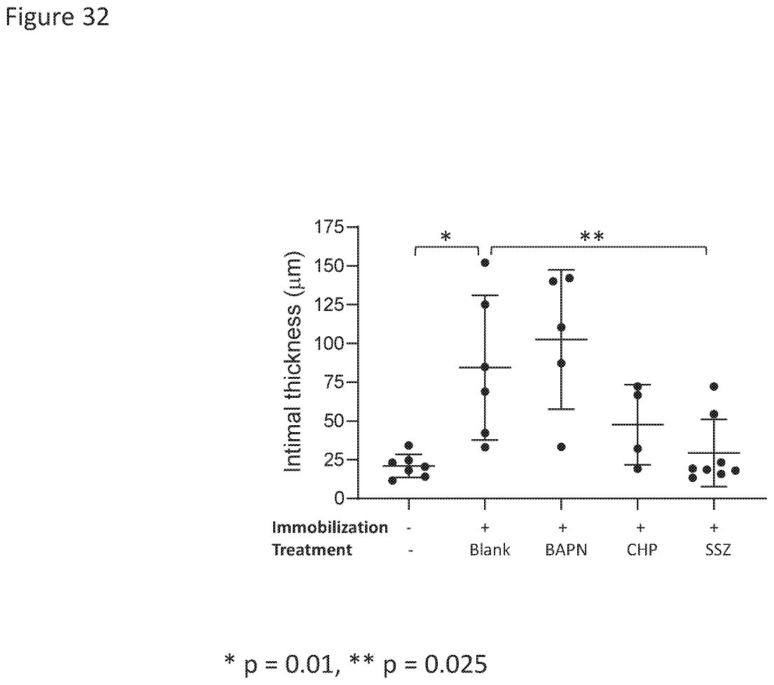

A spanning K-wire fixator held joints in deep flexion for 8 weeks (FIG. 31A). Intra-articular fibrosis was grossly visible in control joints at the time of euthanasia 8 weeks after fixator removal (FIG. 31B). H&E histology revealed thickening of the synovial intima and interstitial fibrosis in posterior capsules from immobilized joints (FIGS. 2C and D). IHC showed a marked increase in α-SMA expression in control versus normal capsules (FIGS. 31E and F). Quantitation of intimal thickness showed a significant increase with immobilization (p=0.01 versus normal), an effect that was inhibited significantly by SSZ (p=0.025 versus control) (FIG. 32).

### Mechanical Testing

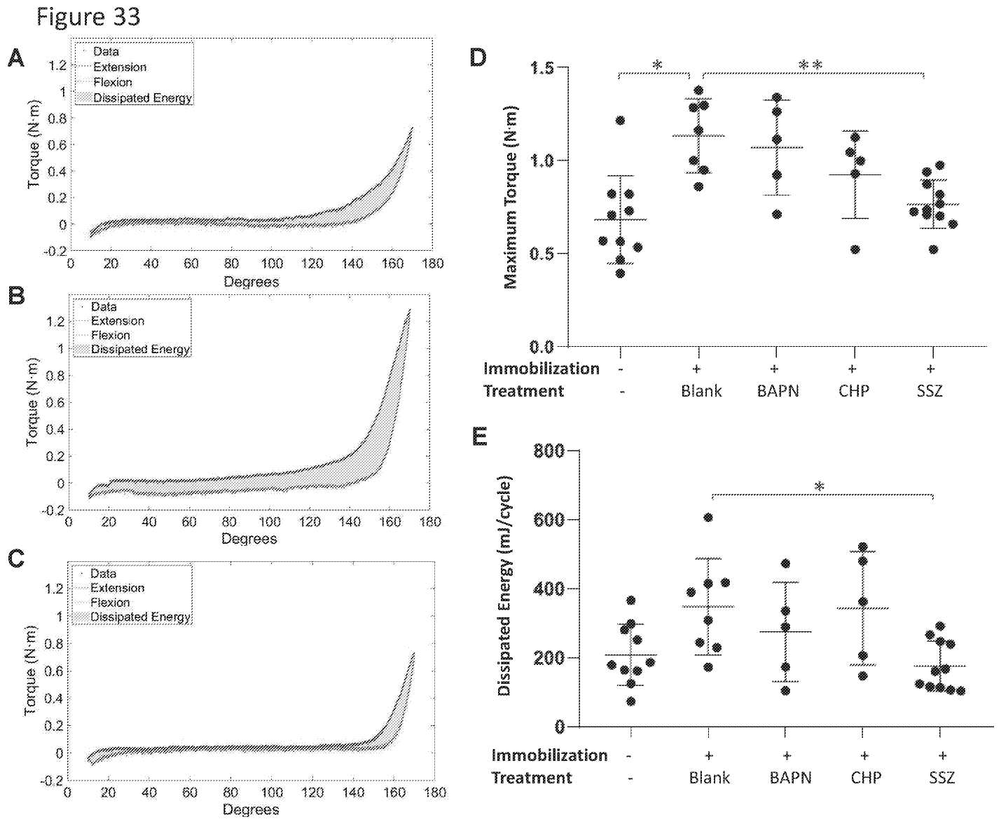

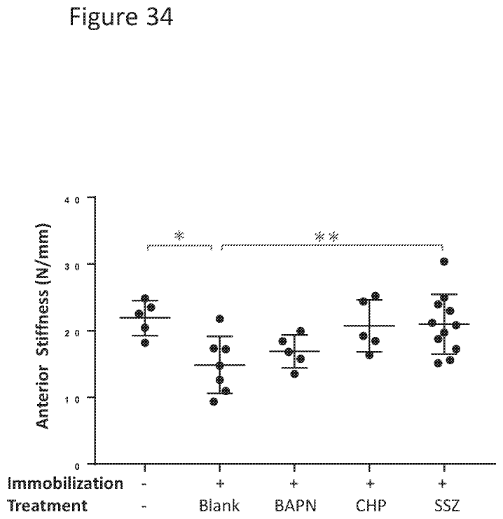

Flexion-extension tests revealed immobilization- and treatment-related differences in the maximum torque required to drive the limbs to full extension and in the shape of the hysteresis loops denoting dissipated energy (FIGS. 33A, B, and C). Maximum torque in control joints was significantly higher than in normal joints (p=0.0008) and joints treated with SSZ were significantly less stiff than controls (p=0.0065) (FIG. 33D). Neither BAPN nor CHP had significant effects on maximum torque. Dissipated energy was significantly reduced only in the SSZ group (p=0.026) (FIG. 33E). Drawer tests showed that anterior stiffness decreased significantly with immobilization (p=0.033) (FIG. 34). This effect was blocked in SSZ-treated joints, which were significantly stiffer than controls (p=0.011).

### In Vitro Cell Analyses

### Collagen Gel Compaction Assay

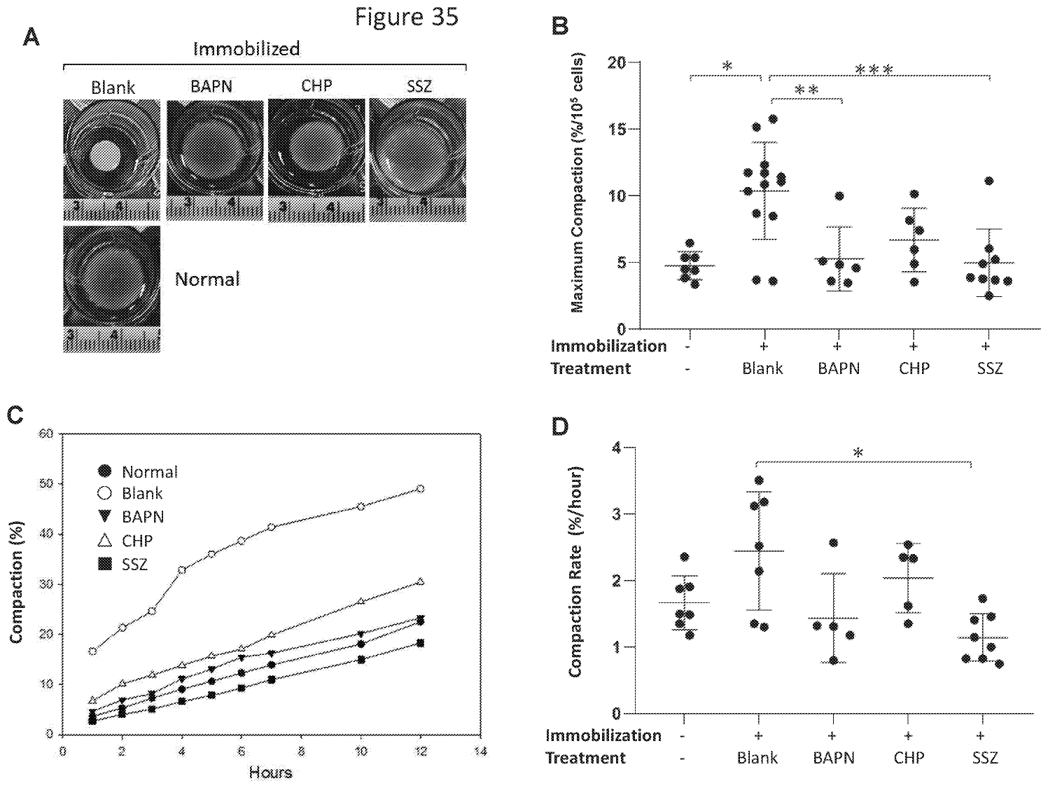

JCFs obtained from normal and variously treated joints were seeded on collagen discs to assess cell contractility. Percent compaction was measured by taking photographs between 0 and 48 hours. Representative images taken at 48 hours are shown (FIG. 35A). Changes in the gel areas were normalized to cell number. Maximum compaction at 48 hours was significantly higher in cells from control joints than in cells from normal joints (p=0.001) (FIG. 35B). Compaction by cells from immobilized joints treated with SSZ or BAPN was significantly lower than in cells from control joints (p=0.005, and 0.0006 respectively) (FIG. 35B). JCFs from SSZ-treated joints also showed a significantly lower compaction rate than cells from control joints over the first 12 hours of incubation (p=0.002) (FIGS. 35C and D). Compaction rates were somewhat lower than control in cells from BAPN- and CHP-treated joints, but the effects were not statistically significant.

### α-SMA Expression

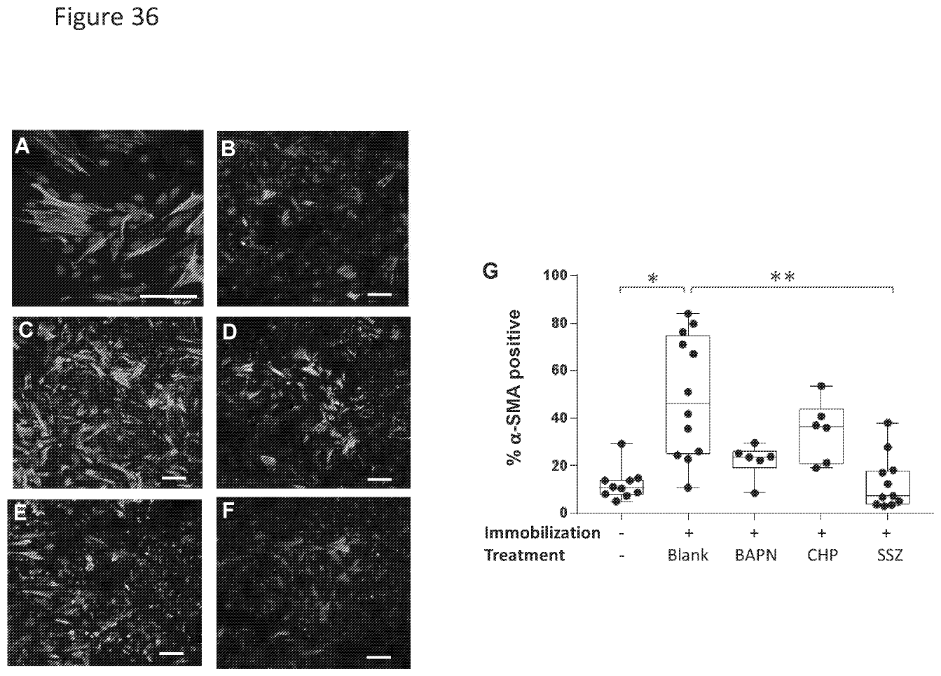

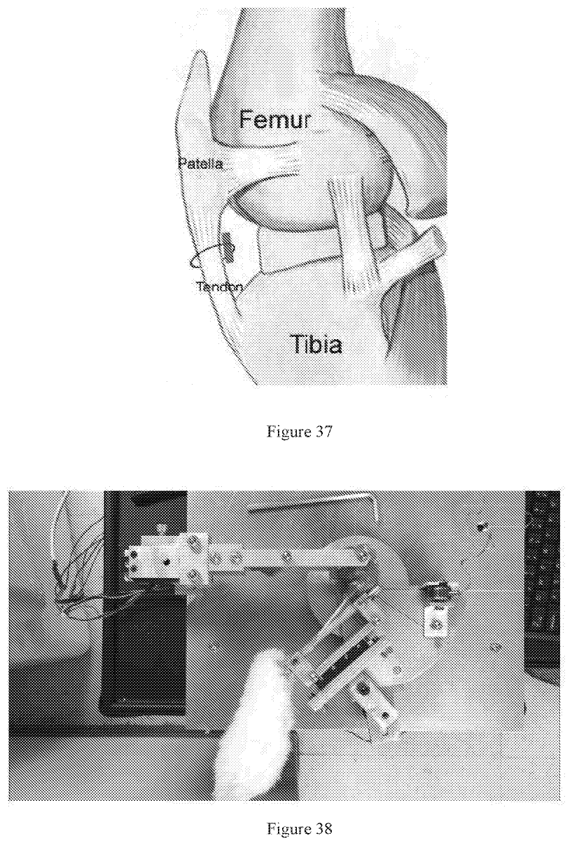

Immunofluorescent detection of α-SMA was performed to determine the percentage of myofibroblasts in the different JCF populations (FIG. 3). α-SMA-positive cells with typical myofibroblast morphology were relatively abundant in JCFs derived from control joints (FIGS. 36A and C). Phalloidin staining revealed an extensive actin stress fiber network in these cells, consistent with myofibroblast cytoskeletal organization. Immobilization significantly enhanced α-SMA expression compared to normal JCFs (p=0.002) (FIG. 36G). The percentage of positives was significantly lower than control in the SSZ group (p=0.0007) and nearly significantly lower in the BAPN group (p=0.058). Expression in the CHP group was only slightly lower than control.

### Correlations Among Outcome Measures

Regression analysis revealed positive correlations between maximum torque, dissipated energy, maximum compaction, compaction rate, and α-SMA expression (Table 4). R² values of 0.5 or greater were observed for many pairwise comparisons. The strongest correlations were between stiffness measures and α-SMA expression (r²=0.64 versus maximum torque and 0.85 versus dissipated energy) and between compaction measures and α-SMA expression (r²=0.81 versus compaction rate and 0.91 versus maximum compaction).

TABLE 4
R2 values for correlations between individual measures

| | Maximum | Dissipated | Compaction | Maximum | % a-SMA |
|---|---|---|---|---|---|
| | Torque | Energy | Rate | Compaction | Positive |
| Maximum Torque | 1.00 | 0.61 | 0,2$ | 0.51 | 0,04 |
| Dissipated Energy | I | 1.00 | 0.73 | 0.59 | 0.85 |
| Compaction Rate | GA | B | too | OJO | 0,81 |
| Maximum | / | / | / | 1.00 | 0.91 |
| Compaction | | | | | |
| % GSMA positive | GA | A | z | n | 1.00 |

### Adverse Events

Unstable fractures and other injuries that prohibited an animal from ambulating, were considered justification for removal from the study. The animals were euthanized after verifying fractures by X-ray (n=8). Fractures occurred at K-wire insertion sites within one week post-op and were not related to treatment. All euthanasia whether scheduled or unscheduled was performed via intravenous injection of euthanasia solution (Euthasol).

### Discussion

Grossly visible fibrotic lesions and histologic evidence of interstitial fibrosis in posterior capsules were associated with alterations in joint stiffness: Maximum extension torque and dissipated energy were significantly higher than normal in control joints, effects that were significantly diminished by treatment with SSZ. In addition, SSZ significantly reduced immobilization-induced intimal thickening.

In vitro assays revealed immobilization-induced increases in maximum compaction and compaction rates. All three drugs mitigated these responses to some degree, but only the SSZ group differed significantly from the control group for both measures. α-SMA expression paralleled compaction activity in that expression increased significantly with immobilization. Although this action was suppressed to varying degrees by all three drugs, only SSZ exerted statistically significant effects. These findings suggest that SSZ has greater therapeutic potential than BAPN and CHP, but we cannot rule out that BAPN and CHP at higher concentrations or in different dosage forms could be as effective as SSZ.

Myofibroblasts under the influence of TGF-β1 resist apoptosis, an effect mediated by nuclear factor kappa light chain enhancer of activated B cells (NF-κB). SSZ has been shown to overcome this resistance by blocking NF-κB activation (Oakley et al., 2005; Abe et al., 2012; Elsharkawy et al., 2005; Oakley et al., 2009). Myofibroblast apoptosis has also been shown to be down-regulated by adhesion to stiff substrates via a Rho-associated protein kinase-mediated mechanotransduction pathway (Van De Water et al., 2013; Junker et al., 2008). Reducing substrate tension leads to rapid induction of BCL-2/cytochrome C-induced apoptosis (Hinz, 2016; Hinz et al., 2001). BAPN and CHP could induce apoptosis through this pathway by degrading the ability of myofibroblasts to sustain the high rates of collagen matrix synthesis required to maintain stiff extracellular matrices. Trends toward reduced joint stiffness, contractile activity, and α-SMA expression in the BAPN and CHP groups are consistent with this hypothesis.

Drugs were encapsulated in PLGA pellets to localize delivery to the fibrotic joint. PLGA is a bioresorbable polymer that has a long track record for use in prolonging drug release in humans (Leelakanok et al., 2018). Pellet delivery allowed drug levels to be steadily maintained for 2-3 weeks, obviating the need for multiple intra-articular injections while reducing toxicity to normal joint tissues. In that regard, drawer test results suggested that pellets carrying SSZ did not lead to A-P plane laxity, indicating that the treatments did not impair ACL function (Tochigi et al., 2011) and in vitro tests showed that the pellet forms of the drugs was not cytotoxic to normal capsule fibroblasts.

Regression analysis revealed positive correlations between mechanical measures (maximum extension torque and dissipated energy) and among biologic measures (maximum compaction, compaction rate, and α-SMA expression). In addition, there was a high degree of correlation between mechanical and biologic measures, suggesting linkage between capsule cell phenotypes and joint stiffness.

The initial injury to the posterior capsule described by others was omitted in order to generate a less severe form of disease amenable to treatment testing. It was reasoned that under the circumstances, mild stiffening of the posterior capsule would be most apparent at maximum extension, an assumption borne out by findings of statistically robust effects of immobilization and treatment on peak stresses at 170°. Although SSZ reversed signs of fibrosis under these narrow circumstances, its efficacy under more severe conditions remains unclear.

In conclusion, this study confirms that arthrofibrosis/capsule contracture can be resolved with medications delivered to the joint space, a strategy that may augment or replace current physical and surgical interventions.

### Example 4

Joint stiffness due to fibrosis/capsule contracture is a seriously disabling complication of articular injury that surgical interventions often fail to completely resolve. Fibrosis/contracture is associated with the abnormal persistence of myofibroblasts, which over-produce and contract collagen matrices. It was hypothesized that intra-articular therapy with drugs targeting myofibroblast survival (sulfasalazine), or collagen production (beta-aminopropionitrile and cis-hydroxyproline), would reduce joint stiffness in a rabbit model of fibrosis/contracture. Drugs were encapsulated in poly[lactic-co-glycolic] acid pellets, and implanted in joints after fibrosis/contracture induction. Capsule alpha-smooth muscle actin (α-SMA) expression and intimal thickness were evaluated by immunohistochemistry and histomorphometry respectively. Joint stiffness was quantified by flexion-extension testing. Drawer tests were employed to determine if the drugs induced cruciate ligament laxity. Joint capsule fibroblasts were tested in vitro for contractile activity and α-SMA expression. Stiffness in immobilized joints treated with blank pellets (control) was significantly higher than in non-immobilized, untreated joints (normal) (p=0.0008), and higher than in immobilized joints treated with sulfasalazine (p=0.0065). None of the drugs caused significant cruciate ligament laxity. Intimal thickness was significantly lower than control in the normal and sulfasalazine-treated groups (p=0.010 and 0.025 respectively). Contractile activity in cells from controls was significantly increased versus normal (p=0.001). Sulfasalazine and beta-aminopropionitrile significantly inhibited this effect (p=0.005 and 0.0006 respectively). α-SMA expression was significantly higher in control versus normal (p=0.0021) and versus sulfasalazine (p=0.0007). These findings support the conclusion that sulfasalazine reduced stiffness by clearing myofibroblasts from fibrotic joints.

The results provide proof that established joint stiffness can be resolved non-surgically.

### Example 5

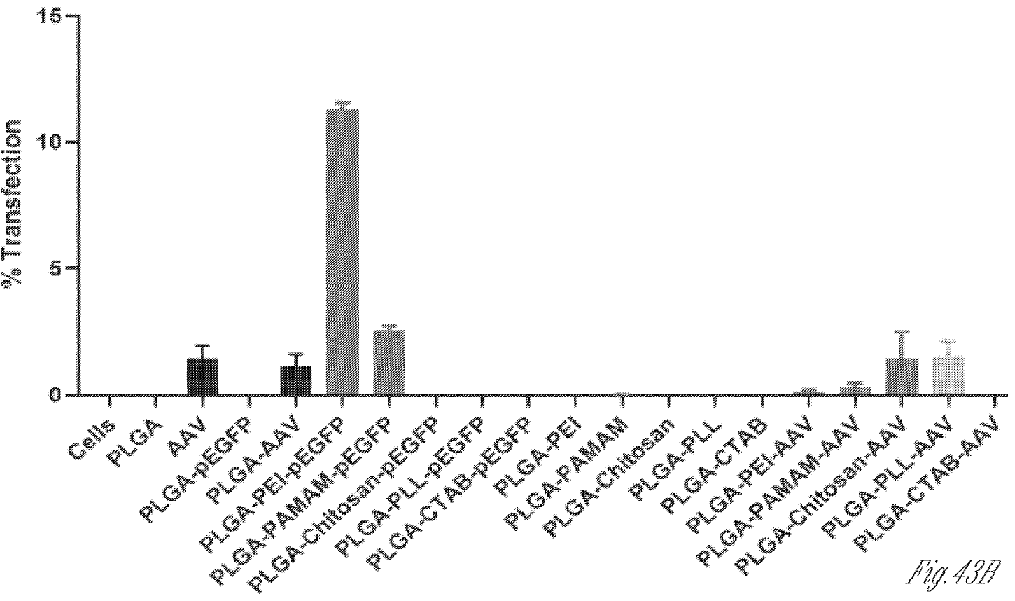

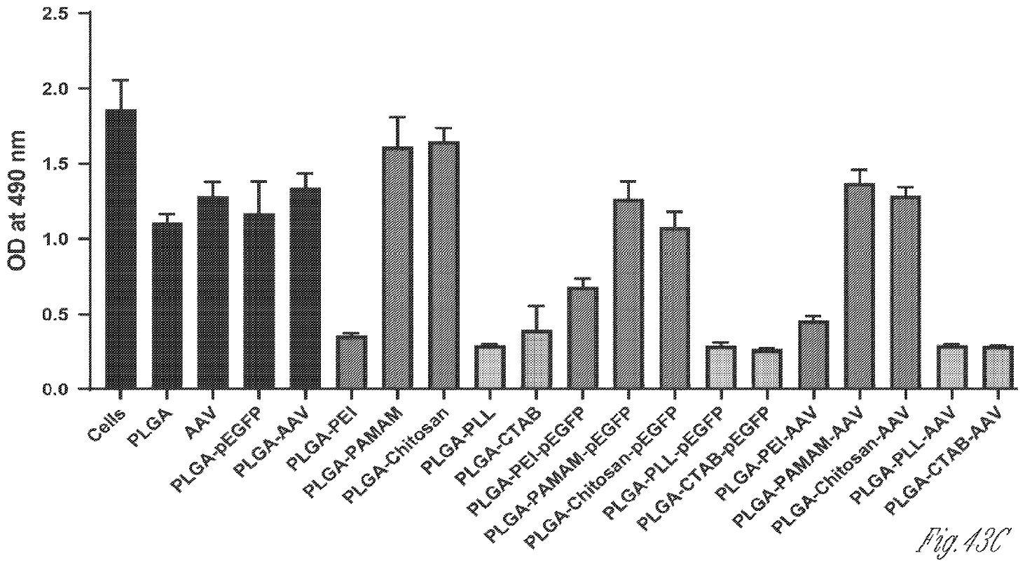

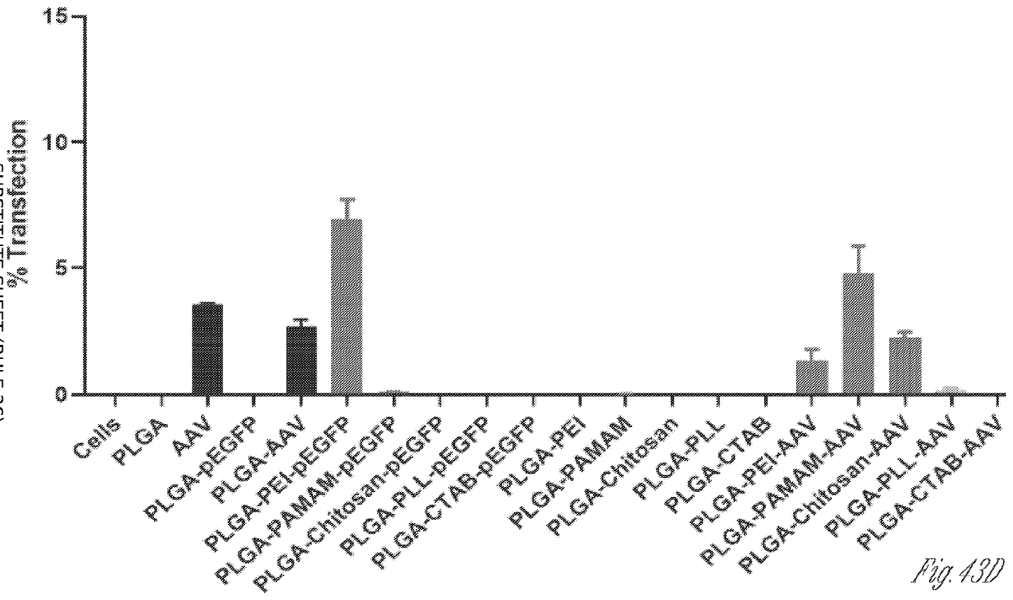

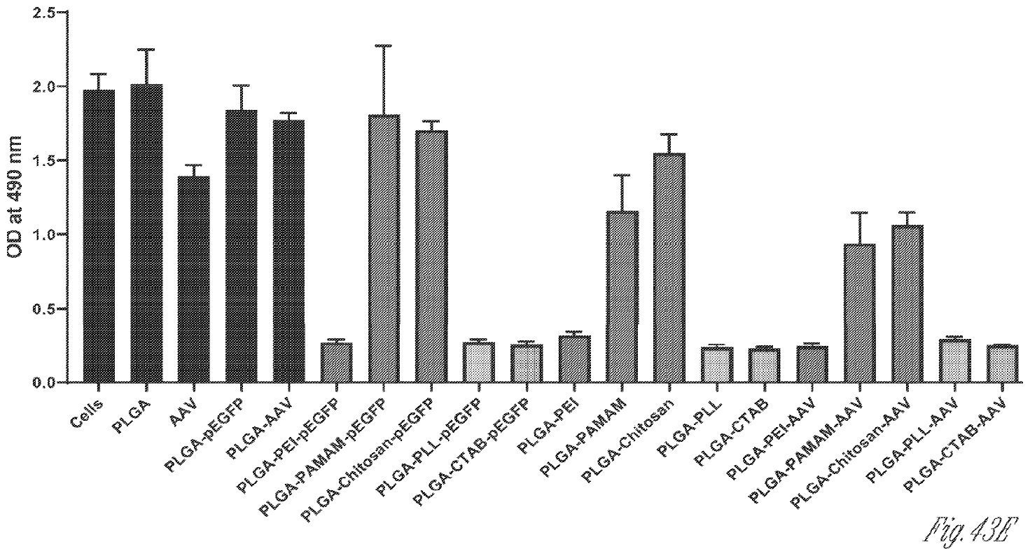

Nanoparticles of PLGA were fabricated by a W/O emulsion method (FIG. 41). Typically, a PLGA solution of 10 mg/mL in acetone is added dropwise to 0.1% PVA solution of 15 mL and stirred for 15 min. The emulsion was subjected to rotary evaporator to remove the solvent and the hardened particles were centrifuged at 500 g for 30 min in Amicon tubes. The particles were washed 4 times and then lyophilized for 48 h.

For preparation of cationic nanoparticles, different cationic agents were introduced in the process at different steps. The PEI and PAMAM branched structures were mixed with PLGA during the synthesis to fabricate PLGA-PEI and PLGA-PAMAM nanoparticles. The CTAB was mixed with PVA solution to form PLGA-CTAB nanoparticles. The chitosan and PLL were used post synthesis of PLGA nanoparticles to form a surface coating. The chitosan and PLL were mixed with the PLGA nanoparticles post synthesis. The prepared cationic nanoparticles were tested for transfection efficiency.

### Particle Size Evaluation

The particle size and zeta potential of the prepared particles was characterized using NanoZS, Malvern, USA. Table 5 showed the particle size, polydispersity index and zeta potential of the as fabricated cationic PLGA nanoparticles.

TABLE 5
Characterization of Cationic Nanoparticles
″os
PLGA137+2.00.07+0.03-15+0.8
PUGAP2//320/*
PLSAPAMAM1211014001/36211
PLGA Chitosan427 ±23 0,56840,14 4212

### DNA Complexation

The fabricated cationic nanoparticles were tested for their complexation with plasmid DNA (pDNA). The complexes were formed by mixing of cationic nanoparticles of 0.5 mL and 25 micrograms of plasmid DNA and incubated for 20 min. The complexes were centrifuged. The pDNA in the supernatant was quantified using Nanodrop. Table 6 showed the results of DNA complexation.

TABLE 6
DNA complexation of cationic nanoparticles with EGFP plasmid (pEGFP)

| ooaopoaoqopsg | ″gapeee |
|---|---|
| _990 | 25t112.521+0 9 |
| PLGA5 | |
| PLGAPAS | 111 256 |
| RGAMMMS | <=<== |
| | / --/ - |
| PLGA-Chitosan ⁵ | 33+112.527±1 |
| Gs | 34*112.546±10 |
| 02g/s | 274312.542+10 |

PLGA-PEI only showed higher complexation compared to other cationic pDNA complexes.

In Vitro Study

The transfection efficiency of the cationic nanoparticles was evaluated in HEK293T, Rabbit Synovial fibroblasts and Bovine chondrocytes. Briefly, 15000 cells were seeded in 48 well plate and incubated for 24 h for cell attachment. After incubation, the spent media was removed and fresh media mixed with 100 μg of nanoparticles complexed with 2 μg of pEGFP plasmid gene for 20 min per well. As comparison another group of associated adenovirus (AAV) was complexed with the nanoparticles. 1×10⁶ vg were complexed with cationic nanoparticles and given to each well. PLGA-PEI-pEGFP showed higher transfection efficiency in HEK293T and rabbit synovial fibroblasts followed by PLGA-PAMAM in HEK293T cells.

Ex-Vivo Study

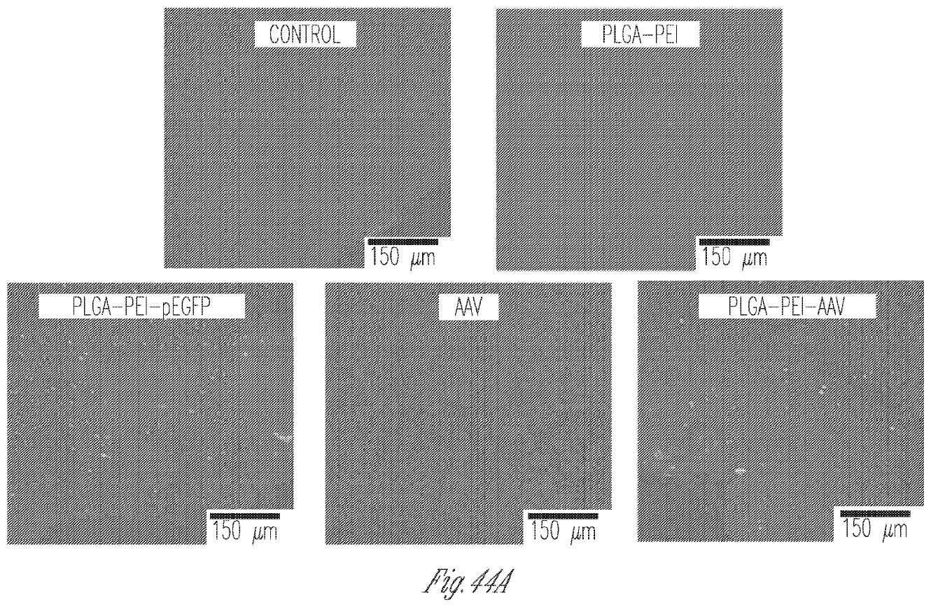

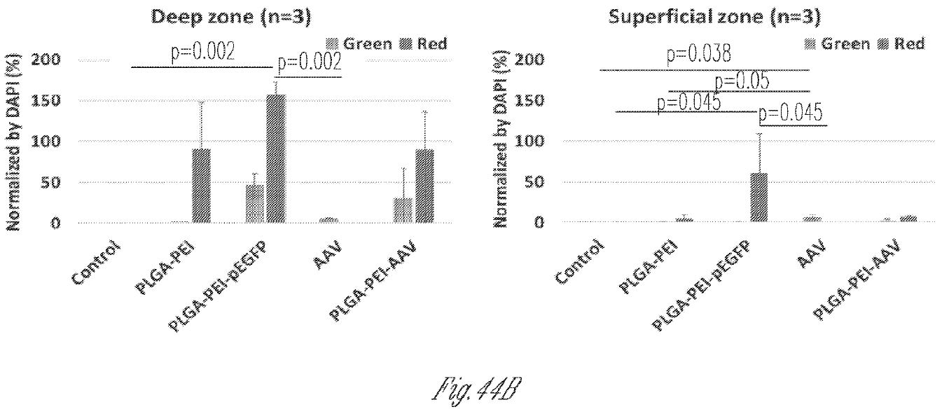

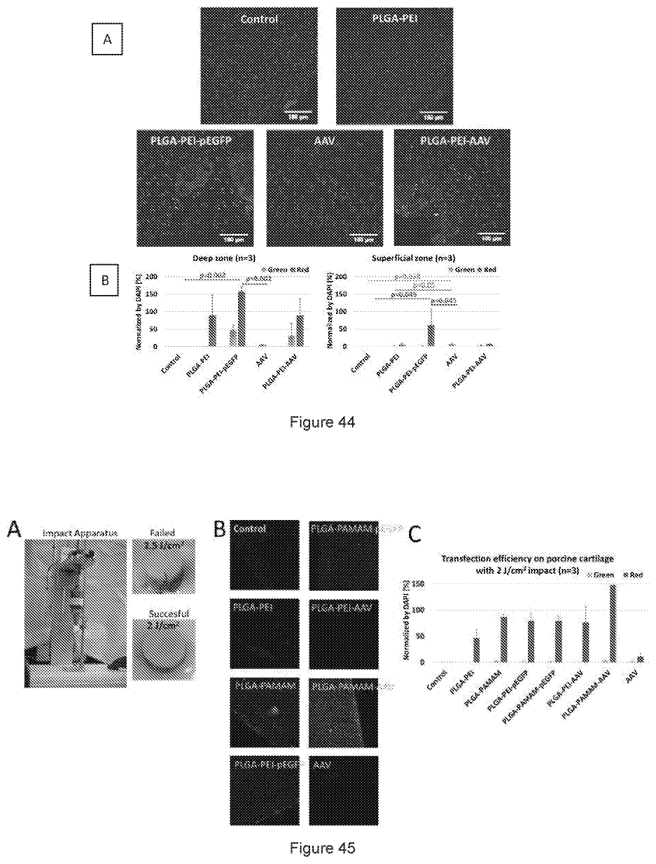

The porcine chondral plugs and synovium were procured from the local butcher shop. The explants were washed thrice with sterile PBS and transferred to DMEM:F12 media supplemented with antibacterial and antifungal solutions. The explants were divided into 5 groups: Control, PLGA-PEI, PLGA-PEI-pEGFP, AAV and PLGA-PEI-AAV. The explants were cultured for 48 h and the transfection of the nanoparticles was evaluated by Confocal microscopy (FIG. 44). To visualize the nanoparticle entry into the cells of the explant, the nanoparticles were encapsulated with Rhodamine B dye. The transfection in the cells was observed with PLGA-PEI-pEGFP and PLGA-PEI-AAV only. There is no transfection AAV group within 48 h. The transfection was occurred mostly in the deep zone of the tissue and none was observed in the superficial zone. The transfection depth was around 0.62 mm for PLGA-PEI-pEGFP signifies the nanoparticle capability to be a robust and effective transfection agent.

The defective cartilage is worn out and degraded. To mimic the defective cartilage, the porcine cartilage samples of 8 mm procured from the local butcher were subjected to impact of 2 J/cm2 (FIG. 45A). After impact, the explants were fixed in the agar gel in a 24 plate under sterile conditions before transfection. The explants were supplemented with DMEM:F12 media. The explants of n=3 were divided into 8 groups: Control, PLGA-PEI, PLGA-PAMAM, PLGA-PEI-pEGFP, PLGA-PAMAM-pEGFP, PLGA-PEI-AAV, PLGA-PAMAM-AAV and AAV. The nanoparticles with and without complexation of AAV and pEGFP were dispersed in media and provided to the explants. The explants were cultured for 48 h and the images were taken under confocal microscope (FIG. 45B).

The results show there is no significant expression of pEGFP across groups in 48 h. However, the uptake of PLGA-PEI and PLGA-PAMAM nanoparticles was significant. This shows that the expression rates are different in the healthy and impact cartilage tissues.

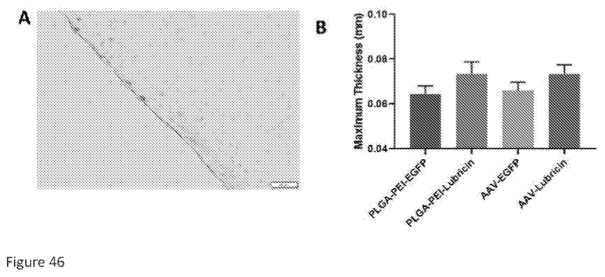

FIG. 46A is an example of lubricin IHC in rabbit cartilage. The antibody labels the cartilage surface, and some superficial chondrocytes (red). “Maximum Thickness” is a measure of how deep the staining extends below the surface, which tends to increase with PRG4 gene transfer. For each animal there were sections taken from the lateral and medial condyles and lateral and medial tibial plateaus (4 sections/joint). The values are averages of all 4 compartments. Safranin-O staining assesses cartilage health.

Thus, the data show that a non-viral formulation and a viral formulation are effective in rabbit cartilage.

### REFERENCES

Abbas et al., J. Pharm. Sci., 97:_(2008).
Abdul et al., Sci. Rep., 5:16469 (2015).
Abe et al., Eur. J. Dermatol., 22:46 (2012).
Arunakul et al., J. Orthop. Res., 31:PMC5113956 (2013).
Atluri et al., Acs Biomater. Sci. Eng., 2:1097 (2016).
Bulut et al., J. Invest. Sur., 17:211 (2004).
Canelon & Wallace, PLoS One, 11:e0166392 (2016).
Cheuy et al., J. Arthroplasty, 12:2604 (2017).
Coleman et al., Sci. Translational Med., _:_(2017).
Danyuo et al., J. Mater. Sci. Mater Med., 28:61 (2017).
Darby et al., Clin. Cosmet. Investig. Dermatol., 7:301 (2014).
Daya & Berns, Clin. Microbiol. Rev., 21:PMC2570152 (2008).
Dean et al., Arthrosc. Tech., 5:e495 (2016).
D'Mello et al., AAPS J., 19:PMC5214458 (2017).
Elsaid et al., Arthritis Rheum., 52:_(2005).
Elsaid et al., Arthritis Rheum., 56:_(2007).
Elsharkawy et al., Apoptosis, 10:927 (2005).
Engel et al., Proc. Natl. Acad. Sci. USA, 103:15546 (2006).
Flannery et la., Arthritis Rheum., 60:_(2009).
Flannery, Curr. Drug Targets, 11:_(2010).
Freeman et al., Fibrogenesis Tissue Repair, 3:17 (2010).
Furlow & Peacock, Ann. Surg., 165:442 (1967).
Goetz et al., J. Orthop. Res., 35:PMC5148713 (2017).
Goetz et al., Osteoarthritis Cartilage, 23:_(2015).
Grinnell & Ho, Exp. Cell Res., 273:248 (2002).
Haller et al., Bone Joint J., 97-B:109 (2015).
Hildebrand et al., J. Orthop. Res., 22:313 (2004).
Hinz et al., Am. J. Pathol., 1:1009 (2001).
Hinz, Exp. Eye Res., L42:56 (2016).
Intra & Salem, J. Drug Target, 19:PMC5258116 (2011).
Jay & Waller, Matrix Biol., 39:_(2014).
Jay et al., Arthritis Rheum., 56:PMC2688668 (2007).
Jay et al., Arthritis Rheum., 62:PMC2921027 (2010).
Jones et al., Arthritis Rheum., 60:_(2009).
Junker et al., Burns, 34:942 (2008).
Kim et al., Am. J. Pathol., 166:1017 (2005).
Kornuijt et al., Musculoskelet. Surg., 102:223 (2018).
Ledwozyw, Acta Physiol. Hung., 83:91 (1995).
Leelakanok et al., J. Pharm. Sci., 107:513 (2018).
Magit et al., J. Am. Acad. Orthop. Surg., 15:682 (2007).
Manrique et al., J. Knee Surg., 28:119 (2015).
McCombe et al., Clin. Orthop. Relat. Res., 451:251 (2006).
Nesterenko et al., J. Orthop. Res., 27:1028 (2009).
Oakley et al., Gastroenterology., 128:108 (2005).
Oakley et al., Gastroenterology., 136:2334 (2009).
Rhee et al., J. Clin. Invest., 115:PMC548698(2005).
Sanders et al., Knee Surg. Sports Traumatol. Arthrosc., 25:532 (2017).
Sawhney & Howard, Cell Motil. Cytoskeleton, 58:175 (2004).
Stephenson et al., Curr. Med. Res. Opin., 26:1109 (2010).
Steplewski et al., J. Orthop. Res., 35:1038 (2017).
Tiede et al., Ann. Anat., 191:33 (2009).
Tochigi et al., J. Bone Joint Surg. Am., 93:640 (2011).
Trudel et al., Arch. Phys. Med. Rehabil., 84:1350 (2003).
Unterhauser et al., Arch. Orthop. Trauma Surg., 12:585 (2004).
Usher et al., Bone Res., 7:9 (2019).
Van De Water et al., Adv. Wound Care (New Rochelle), 2:122 (2013).
Vaseenon et al., J. Orthop. Res., 22:PMC3700429 (2011).
Wakatsuki & Elson., Biophys. Chem. 100:593 (2003).
Wipff et al., J. Cell Biol., 179:1311 (2007).
Yuda & McCulloch, SLAS Discov., 23:132 (2018).
Zhang et al., Histol. Histopathol., 33:27 (2018).
Zhang et al., J. Microencapsul., 25:_(2008).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A composition comprising nanoparticles comprising isolated nucleic acid comprising nucleic acid encoding a mammalian lubricin and a cationic polymer.

2. The composition of claim 1 wherein the nucleic acid encodes a lubricin having at least 80% amino acid sequence identity to any one of SEQ ID Nos. 1-6.

3. (canceled)

4. The composition of claim 1 wherein the cationic polymer comprises a cationic peptide, a linear or branched synthetic polymer, a polysaccharide, a natural polymer or an activated or non-activated dendrimer.

5. The composition of claim 4 wherein the cationic peptide comprises polylysine or polyornithine, the cationic linear or branched synthetic polymer comprises polybrene or polyethyleneimine, the cationic polysaccharide comprises cyclodextrin or chitosan or the cationic polymer comprises PEI, PAMAM or chitosan, or a combination thereof.

6-8. (canceled)

9. The composition of claim 1 wherein the nanoparticles are formed of lactic acid, glycolic acid, caproic acid, or combinations thereof.

10. The composition of claim 1 wherein the nanoparticles have an average diameter of about 450 nm to about 700 nm, about 550 nm to about 650 nm, about 100 nm to about 150 nm or about 150 nm to about 200 nm.

11-13. (canceled)

14. The composition of claim 1 wherein the nanoparticles comprise a coating.

15. The composition of claim 14 wherein the coating comprises a polysaccharide or a peptide.

16. The composition of claim 15 wherein the polysaccharide comprises chitosan.

17. The composition of claim 15 wherein the coating comprises PLL.

18. The composition of claim 1 wherein a virus comprises the nucleic acid encoding lubricin.

19. The composition of claim 1 further comprising an anti-fibrotic agent.

20-23. (canceled)

24. The composition of claim 18 wherein the virus is adenovirus, adeno-associated virus, a herpesvirus, a lentivirus or a retrovirus.

25. (canceled)

26. The composition of claim 18 wherein the virus is combined with nanoparticles.

27-28. (canceled)

29. A method to treat PTOA in a mammal, comprising: administering to a joint space in a mammal in need thereof an effective amount of a composition comprising i) nanoparticles comprising isolated nucleic acid encoding a mammalian lubricin and a cationic polymer, ii) a recombinant viral vector comprising nucleic acid encoding a mammalian lubricin optionally in combination with nanoparticles, iii) nanoplexes comprising a cationic polymer and nucleic acid encoding a mammalian lubricin, or iv) nanoparticles comprising nucleic acid encoding a mammalian lubricin.

30. A method to inhibit or treat injury to cartilage in a mammal, comprising administering to a joint space in a mammal in need thereof an effective amount of a composition comprising i) nanoparticles comprising isolated nucleic acid encoding a mammalian lubricin and a cationic polymer, ii) a recombinant viral vector comprising nucleic acid encoding a mammalian lubricin optionally in combination with nanoparticles, iii) nanoplexes comprising a cationic polymer and nucleic acid encoding a mammalian lubricin, or iv) nanoparticles comprising nucleic acid encoding a mammalian lubricin.

31. The method of claim 29 wherein the mammal is a human.

32. The method of claim 30 wherein the joint is an ankle, knee or shoulder joint.

33. The method of claim 29 further comprising administering amobarbital or a derivative thereof or an anti-fibrotic drug.

34-37. (canceled)

38. The method of claim 29 wherein nanoparticles comprising isolated nucleic acid encoding a mammalian lubricin and a cationic polymer are administered.

39-48. (canceled)


SEQUENCE LISTING (6 sequences)

SEQ ID NO: 1 - protein, 1404 aa, Artificial Sequence
  A synthetic sequence
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVHNPTSPPSSKKAPPPSGASQTIKSTTKRSPKPPNKKKTKKVIESEEITEEHSVSENQESSSSSSSSSSSSTIRKIKSSKNSAANRELQKKLKVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTTPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTSDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGETTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

SEQ ID NO: 2 - protein, 1311 aa, Artificial Sequence
  A synthetic sequence
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTTPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTSDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGETTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

SEQ ID NO: 3 - protein, 1270 aa, Artificial Sequence
  A synthetic sequence
MAWKTLPIYLLLLLSVFVIQQVSSQELSCKGRCFESFERGRECDCDAQCKKYDKCCPDYESFCAEVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTTPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTSDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGETTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

SEQ ID NO: 4 - protein, 1404 aa, Artificial Sequence; identical to SEQ ID NO: 1
  A synthetic sequence

SEQ ID NO: 5 - protein, 1361 aa, Artificial Sequence
  A synthetic sequence
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVHNPTSPPSSKKAPPPSGASQTIKSTTKRSPKPPNKKKTKKVIESEEITEVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTTPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTSDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGETTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

SEQ ID NO: 6 - protein, 1363 aa, Artificial Sequence
  A synthetic sequence
MAWKTLPIYLLLLLSVFVIQQVSSQELSCKGRCFESFERGRECDCDAQCKKYDKCCPDYESFCAEVHNPTSPPSSKKAPPPSGASQTIKSTTKRSPKPPNKKKTKKVIESEEITEEHSVSENQESSSSSSSSSSSSTIRKIKSSKNSAANRELQKKLKVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTTPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTSDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGETTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP